(19) United States
(12) Patent Application Publication
(10) Pub. No.: US 2026/0364174 A1
(43) Pub. Date: Nov. 19, 2026

(54) Purpurin Analogs for Use in Photodynamic Therapy

(71) Applicant: RMW Cho Group Limited, Hong Kong, NT (CN)

(72) Inventors: Sebastian M. Marcuccio, Melbourne (AU); Honsue Cho, Melbourne (AU); Christopher D. Donner, Melbourne (AU); Sacha Novakovic, Melbourne (AU)

(73) Assignee: RMW Cho Group Limited, Hong Kong, NT (CN)

(21) Appl. No.: 19/488,451

(22) PCT Filed: May 31, 2024
(86) PCT No.: PCT/GB2024/051422

§ 371 Date: Nov. 26, 2025

(30) Foreign Application Priority Data
May 31, 2023 (GB) 2308145.8

(51) Int. Cl. A61K 41/00; A61K 39/395; A61K 47/32; A61P 35/00; C07D 471/22; C07D 491/22

(52) U.S. Cl. CPC A61K 41/0076; A61K 39/3955; A61K 47/32; A61P 35/00; C07D 471/22; C07D 491/22

(57) ABSTRACT

The present invention relates to purpurin analogues and their pharmaceutically acceptable salts, and compositions comprising purpurin analogues and their pharmaceutically acceptable salts. Purpurin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of purpurin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

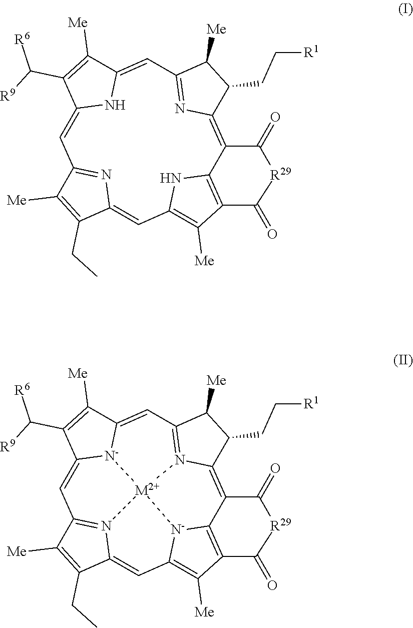

### TECHNICAL FIELD

The present invention relates to purpurin analogues and their pharmaceutically acceptable salts, and compositions comprising purpurin analogues and their pharmaceutically acceptable salts. Purpurin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of purpurin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

The purpurin analogues of the present invention can be analogues of, for example, purpurinimide or purpurin 18, the structures of which are shown below:

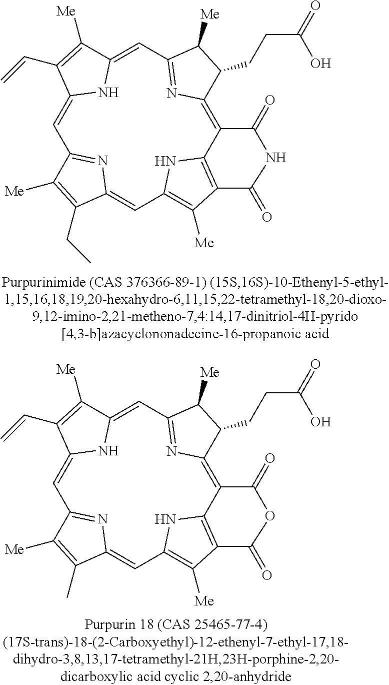 [SMILES: C=Cc1c(C)c2cc3nc(c4c5[nH]c(cc6nc(cc1[nH]2)C(C)=C6CC)c(C)c5C(=O)NC4=O)[C@@H](CCC(=O)O)[C@@H]3C ; C=Cc1c(C)c2cc3nc(c4c5[nH]c(cc6nc(cc1[nH]2)C(C)=C6C)c(C)c5C(=O)OC4=O)[C@@H](CCC(=O)O)[C@@H]3C]

### BACKGROUND ART

Porphyrins and their analogues are known photosensitive chemical compounds, which can absorb light photons and emit them at higher wavelengths. There are many applications for such unique properties and PDT (photodynamic therapy) is one of them.

Presently, there are two generations of photosensitizers for PDT. The first generation comprises heme porphyrins (blood derivatives), and the second for the most part are chlorophyll analogues. The later compounds are known as chlorins and bacteriochlorins.

Chlorin e4 has been shown to display good photosensitive activity. It was indicated that chlorin e4 has a protective effect against indomethacin-induced gastric lesions in rats and TAA- or CCl4-induced acute liver injuries in mice. It was therefore suggested that chlorin e4 may be a promising new drug candidate for anti-gastrelcosis and liver injury protection. WO 2009/040411 suggests the use of a chlorin e4 zinc complex in photodynamic therapy and WO 2014/091241 suggests the use of chlorin e4 disodium in photodynamic therapy.

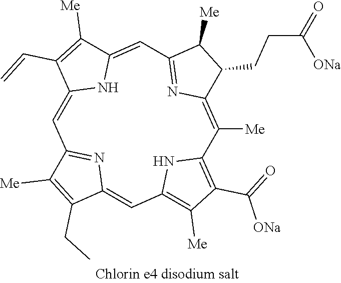

While the conjugation of molecules to triphenylphosphonium cations is known in the literature to enhance delivery to the mitochondria, this is not always guaranteed as demonstrated in a recent paper by Gilson et al (Bioconjugate Chemistry, 2019, vol 30(5), pages 1451-1458). The addition of one triphenylphosphonium cation to the known photodynamic agent chlorin e6 resulted in the derivative accumulating in the lysosomes, while the addition of two triphenylphosphonium cations resulted in distribution to the lysosomes and the mitochondria. The authors concluded that “mitochondrial localized PS did not improve cell killing in this study” and the unconjugated parent chlorin e6 displayed better photodynamic (cell killing) activity than the two triphenylphosphonium conjugated derivatives.

There is an ongoing need for better photosensitizers. There is a need for compounds that have a high singlet oxygen quantum yield and for compounds that have a strong photosensitizing ability, preferably in organic and aqueous media. There is also a need for compounds that have a high fluorescence quantum yield. In addition, there is a need for compounds and/or compositions which have a higher phototoxicity, a lower dark toxicity, good stability (such as increased resistance to destruction by ROS), good solubility, and/or are easily manufactured and/or purified.

### SUMMARY OF THE INVENTION

A first aspect of the present invention provides a compound of formula (I) or a complex of formula (II):

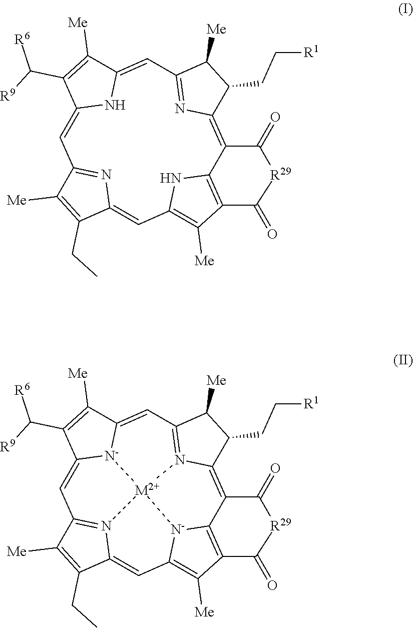

or a pharmaceutically acceptable salt thereof, wherein:
R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
n is 1, 2, 3, 4, 5 or 6;
X is a halo group;
Y is a counter anion;
Z is a counter cation; and
M²⁺ is a metal cation;
provided that the compound of formula (I) or the complex of formula (II) comprises at least one group selected from —R^{α}—[P(R⁵)₃]Y, —R^{α}—[R⁷]Y, —R^{α}—[P(R⁵)₂(R^{5′})] or —R^{α}—[R^{7′}].

The first aspect of the present invention also provides a compound of formula (I) or a complex of formula (2):

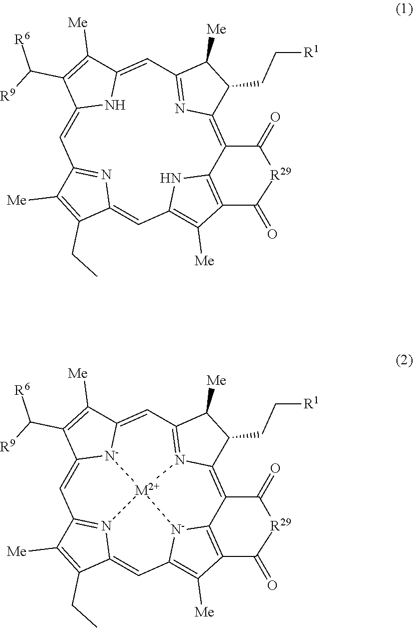

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
n is 1, 2, 3, 4, 5 or 6;
X is a halo group;
Y is a counter anion;
Z is a counter cation; and
M²⁺ is a metal cation;
provided that the compound of formula (I) or the complex of formula (2) comprises at least one group selected from —R^{α}—[P(R⁵)₃]Y, —R^{α}—[R⁷]Y, —R^{α}—[P(R⁵)₂(R^{5′})] or —R^{α}—[R^{7′}].

A second aspect of the present invention provides a compound of formula (I) or a complex of formula (II) according to the first aspect of the invention, for use in medicine.

In the context of the present specification, a “hydrocarbyl” substituent group or a hydrocarbyl moiety in a substituent group only includes carbon and hydrogen atoms but, unless stated otherwise, does not include any heteroatoms, such as N, O, S, P or Se in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O, S, P or Se in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₆₀ hydrocarbyl group, more typically a C₁-C₄₀ hydrocarbyl group, more typically a C₁-C₂₀ hydrocarbyl group. More typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group. Typically an alkylene group is a C₁-C₄₂ alkylene group. More typically an alkylene group is a C₁-C₃₂ alkylene group, or a C₁-C₂₂ alkylene group, or a C₁-C₁₂ alkylene group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds.

Examples of alkynyl groups/moieties include ethynyl, propargyl, but-1-ynyl and but-2-ynyl. Typically an alkynyl group is a C₂-C₁₂ alkynyl group. More typically an alkynyl group is a C₁-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O, S, P or Se in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O, S, P or Se in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

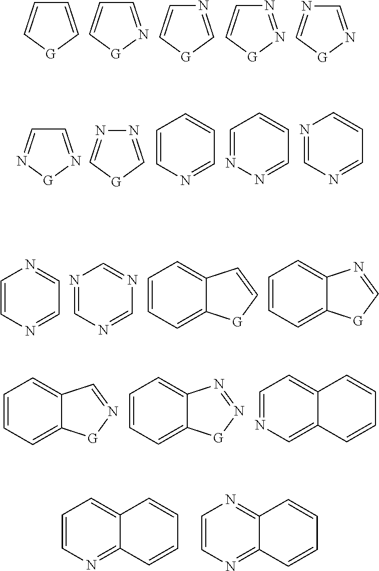

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety (such as —R^{β}):
(i) each hydrogen atom may optionally be replaced by a monovalent substituent independently selected from halo; —CN; —NO₂; —N₃; —R^{x}; —OH; —OR^{x}; —R^{y}-halo; —R^{y}—CN; —R^{y}—NO₂; —R^{y}—N₃; —R^{y}—R^{x}; —R^{y}—OH; —R^{y}—OR^{x}; —SH; —SR^{x}; —SOR^{x}; —SO₂H; —SO₂R^{x}; —SO₂NH₂; —SO₂NHR^{x}; —SO₂N(R^{x})₂; —R^{y}—SH; —R^{y}—SR^{x}; —R^{y}—SOR^{x}; —R^{y}—SO₂H; —R^{y}—SO₂R^{x}; —R^{y}—SO₂NH₂; —R^{y}—SO₂NHR^{x}; —R^{y}—SO₂N(R^{x})₂: —NH₂; —NHR^{x}; —N(R^{x})₂; —N⁺(R^{x})₃; —R^{y}—NH₂; —R^{y}—NHR^{x}; —R^{y}—N(R^{x})₂; —R^{y}—N⁺(R^{x})₃; —CHO; —COR^{x}; —COOH; —COOR^{x}; —OCOR^{x}; —R^{y}—CHO; —R^{y}—COR^{x}; —R^{y}—COOH; —R^{y}—COOR^{x}; or —R^{y}—OCOR^{x}; and/or
(ii) any two hydrogen atoms attached to the same carbon atom may optionally be replaced by a r-bonded substituent independently selected from oxo (═O), ═S, ═NH, or ═NR^{x}; and/or
(iii) any two hydrogen atoms attached to the same or different atoms, within the same optionally substituted group or moiety, may optionally be replaced by a bridging substituent independently selected from —O—, —S—, —NH—, —N(R^{x})—, —N⁺(R^{x})₂— or —R^{y}—;
   wherein each —R^{y}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{x} groups; and
   wherein each —R^{x} is independently selected from a C₁-C₆ alkyl, C₂-C₆ alkenyl, C₂-C₆ alkynyl or C₂-C₆ cyclic group, or wherein any two or three —R^{x} attached to the same nitrogen atom may, together with the nitrogen atom to which they are attached, form a C₂-C₇ cyclic group, and wherein any —R^{x} may optionally be substituted with one or more C₁-C₄ alkyl, C₁-C₄ haloalkyl, —O(C₁-C₄ alkyl), —O(C₁-C₄ haloalkyl), halo, —OH, —NH₂, —CN, or oxo (═O) groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{x})—, —N⁺(R^{x})₂— or —R^{y}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O, S, P or Se in its carbon skeleton, or where reference is made to a carbon atom of a hydrocarbyl or other group being replaced by an N, O, S, P or Se atom, what is intended is that:

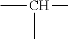

is replaced by

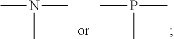

—CH₂— is replaced by —NH—, —PH—, —O—, —S— or —Se—;
—CH₃ is replaced by —NH₂, —PH₂, —OH, —SH or —SeH;
—CH═ is replaced by —N═ or —P═;
CH₂═ is replaced by NH═, PH═, O═, S═ or Se═; or
CH≡ is replaced by N≡ or P≡;
provided that the resultant group comprises at least one carbon atom. For example, methoxy, dimethylamino and aminoethyl groups are considered to be hydrocarbyl groups including one or more heteroatoms N, O, S, P or Se in their carbon skeleton.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O, S, P or Se, are to be counted as carbon atoms when calculating the number of carbon atoms in a Cₓ-C_{y} group. For example, a morpholinyl group is to be considered a C₆ heterocyclic group, not a C₄ heterocyclic group.

The π electrons of the chlorin ring are delocalised and therefore the chlorin ring can be depicted by more than one resonance structure. Resonance structures are different ways of drawing the same compound. Two of the resonance structures of the chlorin ring are depicted directly below:

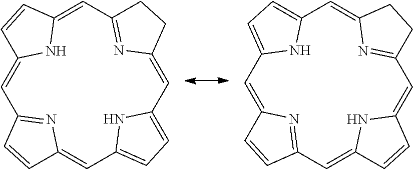 [SMILES: C1=Cc2cc3ccc(cc4nc(cc5ccc(cc1n2)[nH]5)CC4)[nH]3 ; C1=Cc2cc3ccc(cc4nc(cc5ccc(cc1n2)[nH]5)CC4)[nH]3]

Typically a complex comprises a central metal atom or ion known as the coordination centre and a bound molecule or ion which is known as a ligand. In the present specification, the bond between the coordination centre and the ligand is depicted as shown in the complex on the below left (where the attraction between an anionic ligand and a central metal cation is represented by four dashed lines), but equivalently it could be depicted as shown in the complex on the below right (where the attraction between a ligand molecule and a central metal atom is represented by two covalent bonds and two dashed lines):

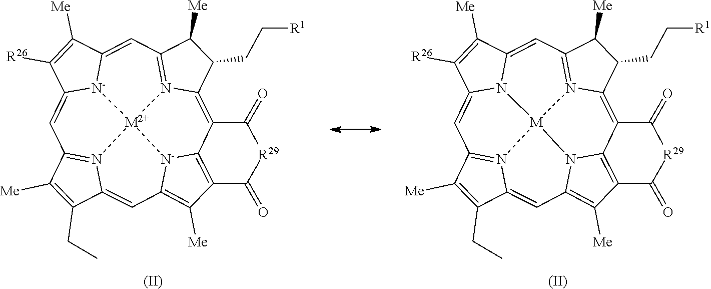

As used herein —[NC₅H₅]Y refers to:

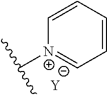

The compound of formula (I) or the complex of formula (II) comprises at least one group selected from —R^{α}—[P(R⁵)₃]Y, —R^{α}—[R⁷]Y, —R^{α}—[P(R⁵)₂(R^{5′})] or —R^{α}—[R^{7′}]. In a preferred embodiment, the compound of formula (I) or the complex of formula (II) comprises at least one group selected from —R^{α}—[P(R⁵)₃]Y or —R^{α}—[P(R⁵)₂(R^{5′})]. In one embodiment, —R²⁶ or —R⁶ comprises —R^{α}—[P(R⁵)₃]Y, —R^{α}—[R⁷]Y, —R^{α}—[P(R⁵)₂(R^{5′})] or —R^{α}—[R^{7′}], preferably —R²⁶ or —R⁶ comprises —R^{α}—[P(R⁵)₃]Y or —R^{α}—[P(R⁵)₂(R^{5′})]. In one embodiment, —R¹ comprises —R^{α}—[P(R⁵)₃]Y, —R^{α}—[R⁷]Y, —R^{α}—[P(R⁵)₂(R^{5′})] or —R^{α}—[R^{7′}], preferably —R¹ comprises —R^{α}—[P(R⁵)₃]Y or —R^{α}—[P(R⁵)₂(R^{5′})]. In one embodiment, —R²⁹ or —R⁹ comprises —R^{α}—[P(R⁵)₃]Y, —R^{α}—[R⁷]Y, —R^{α}—[P(R⁵)₂(R^{5′})] or —R^{α}—[R^{7′}], preferably —R²⁹ or —R⁹ comprises —R^{α}—[P(R⁵)₃]Y or —R^{α}—[P(R⁵)₂(R^{5′})].

In one embodiment of the first or second aspect of the present invention, X is a halo group selected from fluoro, chloro, bromo, or iodo. In one embodiment, X is chloro or bromo.

In one embodiment of the first or second aspect of the present invention, there is provided a compound of formula (I).

In one embodiment of the first or second aspect of the present invention, Y is a counter anion selected from halides (for example fluoride, chloride, bromide, or iodide) or other inorganic anions (for example bisulfate, hexafluorophosphate (PF6), nitrate, perchlorate, phosphate, or sulfate) or organic anions (for example acetate, ascorbate, aspartate, benzoate, besylate (benzenesulfonate), bicarbonate, bis(trifluoromethanesulfonyl)imide (TFSI), bitartrate, butyrate, camsylate (camphorsulfonate), carbonate, citrate, decanoate, edetate, esylate (ethanesulfonate), fumarate, galactarate, gluceptate, gluconate, glutamate, glycolate, hexanoate, β-hydroxybutyrate, 2-hydroxyethanesulfonate, hydroxymaleate, hydroxynaphthoate, isethionate, lactate, lactobionate, malate, maleate, mandelate, mesylate (methanesulfonate), methylsulfate, mucate, napsylate (naphthalene-2-sulfonate), octanoate, oleate, ornithinate, pamoate, pantothenate, polygalacturonate, propanoate, propionate, salicylate, stearate, succinate, tartrate, teoclate, tetrakis[3,5-bis(trifluoromethyl)phenyl]borate (BARF), tetrakis(pentafluorophenyl)borate (F5-TPB), tetraphenylborate (TPB), tosylate (toluene-p-sulfonate), or triflate (trifluoromethanesulfonate)). In one embodiment, Y is fluoride, chloride, bromide, iodide or hexafluorophosphate. In one embodiment, Y is fluoride, chloride, bromide or iodide. In one embodiment, Y is chloride or bromide.

In one embodiment of the first or second aspect of the present invention, Z is a counter cation selected from inorganic cations (for example lithium, sodium, potassium, magnesium, calcium or ammonium cation) or organic cations (for example amine cations (for example choline or meglumine cation) or amino acid cations (for example arginine cation).

In one embodiment of the first or second aspect of the present invention, M²⁺ is a metal cation selected from Zn²⁺, Cu²⁺, Fe²⁺, Pd²⁺ or Pt²⁺. In one embodiment, M²⁺ is Zn²⁺.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂. In one embodiment, —R¹ is selected from —C(O)—OR³, —C(O)—SR³, —C(O)—N(R³)₂ or —C(S)—N(R³)₂. In one embodiment, —R¹ is selected from —C(O)—OR³, —C(O)—SR³ or —C(O)—N(R³)₂.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl (preferably methyl). In one embodiment, —R¹ is selected from —C(O)—OR³, —C(O)—SR³, —C(O)—N(R³)₂ or —C(S)—N(R³)₂, and each —R³ is C₁-C₄ alkyl (preferably methyl). In one embodiment, —R¹ is selected from —C(O)—OR³, —C(O)—SR³ or —C(O)—N(R³)₂, and each —R³ is C₁-C₄ alkyl (preferably methyl). In one embodiment, —R¹ is —C(O)—OR³ and —R³ is C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); —R^{3′} is H or C₁-C₄ alkyl (preferably methyl); and —R² or —R³ is selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]. In one embodiment, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); —R^{3′} is H or C₁-C₄ alkyl (preferably methyl); and —R² or —R³ is selected from —R^{α}—[N(R⁵)₃]Y, —R^{α}—[P(R⁵)₃]Y, or —R^{α}—[R⁷]Y. In one embodiment, —R¹ is selected from —R², —C(O)—OR³, —C(O)—SR³, —C(O)—N(R³)(R^{3′}) or —C(S)—N(R³)(R^{3′}); —R^{3′} is H or C₁-C₄ alkyl (preferably methyl); —R² or —R³ is selected from —R^{α}—[N(R⁵)₃]Y, —R^{α}—[P(R⁵)₃]Y, or —R^{α}—[R⁷]Y; each —R⁵ is independently selected from C₁-C₄ alkyl or phenyl wherein the phenyl is optionally substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is —C(O)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R¹ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In any of the embodiments in the four preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a C₁-C₈ alkyl group optionally substituted with one or more (such as one, two, three, four, five, six, seven or eight) —OH or —OAc groups, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). Typically in these embodiments, —R^{α}— is an unsubstituted C₁-C₆ alkylene group, or an unsubstituted C₁-C₄ alkylene group, or an unsubstituted C₁-C₂ alkylene group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is —C(O)—N(R³)(R^{3′}); wherein —R³ is selected from —R^{α}—H or —R^{α}—OH; —R^{α}— is selected from a C₁-C₁₂ alkylene group, wherein one or more carbon atoms in the backbone of the alkylene group may optionally be replaced by one or more heteroatoms O or S; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is a C₁-C₁₂ alkyl or C₂-C₁₂ alkenyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{β}; —R^{β} is a C₁-C₁₂ alkyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is —C(O)—N(R³)(R^{3′}); wherein —R³ is —R^{β}; —R^{β} is a C₁-C₈ alkyl group optionally substituted with one or more (such as one, two or three) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{Z} is the side chain of a natural amino acid; and —R^{3′} is H or C₁-C₄ alkyl (preferably methyl).

In one embodiment of the first or second aspect of the present invention, —R¹ is selected from —CO—(NR^{zz}—CHR^{z}—CO)ᵥ—N(R^{zz})₂ and —CO—(NR^{zz}—CHR^{z}—CO)ᵥ—OR^{zz}; wherein each —R^{z} is independently selected from the side chains of natural amino acids; each —R^{zz} is independently selected from hydrogen and C₁-C₄ alkyl (preferably methyl); and v is 1, 2, 3, 4, 5, 6, 7 or 8.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R¹ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation). In one embodiment, —R¹ is —C(O)—OR³, wherein —R³ is selected from C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R¹ is —C(O)—N(R³)₂. In one embodiment, —R¹ is —C(O)—N(C₁-C₄ alkyl)(R³) or —C(O)—NHR³. In one embodiment, —R¹ is —C(O)—N(CH₃)(R³) or —C(O)—NHR³. In one embodiment, —R¹ is —C(O)—N(C₁-C₄ alkyl)(R³). In one embodiment, —R¹ is —C(O)—N(CH₃)(R³).

In one embodiment of the first or second aspect of the present invention, —R¹ is selected from —CH₂OR², —CH₂SR², —CH₂S(O)R², —CH₂S(O)₂R², —CH₂N(R²)₂, or —R². In one embodiment, —R¹ is selected from —CH₂OR², —CH₂SR², —CH₂N(R²)₂, or —R². In one embodiment, —R¹ is selected from —CH₂OR², —CH₂SR², or —CH₂N(R²)₂. In one embodiment, —R¹ is selected from —CH₂OR² or —CH₂SR². In one embodiment, —R¹ is —CH₂OR². In one embodiment, —R¹ is —R², and —R² is —R^{α}—X.

In one embodiment of the first or second aspect of the present invention, —R² is selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]. In one embodiment, —R² is selected from —R^{α}—[N(R⁵)₃]Y, —R^{α}—[P(R⁵)₃]Y, or —R^{α}—[R⁷]Y. In one embodiment, —R² is selected from —R^{α}—[N(R⁵)₃]Y, —R^{α}—[P(R⁵)₃]Y, or —R^{α}—[R⁷]Y; each —R⁵ is independently selected from C₁-C₄ alkyl or phenyl wherein the phenyl is optionally substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, —R² is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; and Y is fluoride, chloride, bromide or iodide. Typically in this embodiment, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R² is selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[R⁷]Y. In one embodiment, —R² is selected from —R^{α}—OR^{β}, —R^{α}—SR^{β}, —R^{α}—S(O)R^{β} or —R^{α}—S(O)₂R^{β}. In one embodiment, —R² is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R² is selected from —R^{α}—OR^{β} or —R^{α}—SR^{β}. In one embodiment, —R² is selected from —R^{α}—OR^{β} or —R^{α}—SR^{β}, and —R^{β} is a saccharidyl group.

In one embodiment of the first or second aspect of the present invention, —R² is selected from —C(O)R⁴, —C(O)—OR⁴, —C(O)—SR⁴, —C(O)—N(R⁴)₂, —C(S)—OR⁴, —C(S)—SR⁴ or —C(S)—N(R⁴)₂. In one embodiment, —R² is selected from —C(O)R⁴, —C(O)—OR⁴, —C(O)—SR⁴, —C(O)—N(R⁴)₂ or —C(S)—N(R⁴)₂. In one embodiment, —R² is selected from —C(O)R⁴, —C(O)—OR⁴, —C(O)—SR⁴ or —C(O)—N(R⁴)₂.

In one embodiment of the first or second aspect of the present invention, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is H or C₁-C₄ alkyl (preferably methyl).

An —R^{4′} group refers to an —R⁴ group attached to the same atom as another —R⁴ group. —R⁴ and —R^{4′} may be the same or different. Preferably —R⁴ and —R^{4′} are different.

In one embodiment of the first or second aspect of the present invention, —R² is —C(O)—N(R⁴)₂. In one embodiment, —R² is —C(O)—N(C₁-C₄ alkyl)(R⁴). In one embodiment, —R² is —C(O)—N(CH₃)(R⁴).

In one embodiment of the first or second aspect of the present invention, —R²⁶ is selected from —CH₂R⁶, —CH₂CH₃ or —CH═CH₂. In one embodiment, —R²⁶ is —CH₂R⁶.

In one embodiment of the first or second aspect of the present invention, —R⁶ is selected from —OR², —N(R²)₂, —SR², —S(O)R² or —S(O)₂R². In one embodiment, —R⁶ is selected from —OR², —SR², —S(O)R² or —S(O)₂R². In one embodiment, —R⁶ is selected from —OR² or —SR². In one embodiment, —R⁶ is —OR².

In one embodiment of the first or second aspect of the present invention, —R⁶ is selected from —OR², —N(R²)₂, —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]. In one embodiment, —R⁶ is selected from —OR², —SR², —S(O)R² or —S(O)₂R², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]. In one embodiment, —R⁶ is selected from —OR² or —SR², and —R² is selected from —H, —C(O)R⁴, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}].

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]; —R^{4′} is selected from hydrogen or C₁-C₄ alkyl; and —R⁴ is selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]. In one embodiment, —R⁶ is selected from —OR², —N(R²)(R^{2′}), —SR², —S(O)R² or —S(O)₂R²; —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]; —R^{4′} is selected from hydrogen or C₁-C₄ alkyl; —R⁴ is selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —CO₂, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —CO₂⁻ and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate). In one embodiment, —R⁶ is selected from —OR² or —N(R²)(R^{2′}); —R^{2′} is selected from hydrogen, C₁-C₄ alkyl or —CO₂(C₁-C₄ alkyl); —R² is selected from —C(O)R⁴, —C(O)—OR⁴, —C(O)—N(R⁴)(R^{4′}), —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]; —R^{4′} is selected from hydrogen or C₁-C₄ alkyl; —R⁴ is selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —CO₂, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —CO₂⁻ and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, —R⁶ is selected from —O—C(O)R¹⁴, —N(R¹²)—C(O)R¹⁴, —O—C(O)—OR¹⁴, —N(R¹²)—C(O)—OR¹⁴, —O—C(O)—N(R¹²)(R¹⁴), or —N(R¹²)—C(O)—N(R¹²)(R¹⁴); —R¹² is selected from hydrogen or C₁-C₃ alkyl (preferably hydrogen or methyl); —R¹⁴ is selected from —R^{α}—[P(R⁵)₃]Y, —R^{α}—[R⁷]Y, —R^{α}—[P(R⁵)₂(R^{5′})] or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —CO₂⁻, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —CO₂⁻ and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{α}—OR^{β}, —R^{α}—SR^{β}, —R^{α}—S(O)R^{β} or —R^{α}—S(O)₂R^{β}; and optionally —R^{β} is a saccharidyl group. In one embodiment, —R⁶ is selected from —OR², —SR², —S(O)R² or —S(O)₂R², and —R² is selected from —R^{α}—OR^{β}, —R^{α}—SR^{β}, —R^{α}—S(O)R^{β} or —R^{α}—S(O)₂R^{β}, and optionally —R^{β} is a saccharidyl group. In one embodiment, —R⁶ is selected from —OR², —SR², —S(O)R² or —S(O)₂R², and —R² is selected from —R^{α}—OR^{β} or —R^{α}—SR^{β}, and optionally —R^{β} is a saccharidyl group. In one embodiment, —R⁶ is selected from —OR² or —SR², and —R² is selected from —R^{α}—OR^{β}, —R^{α}—SR^{β}, —R^{α}—S(O)R^{β} or —R^{α}—S(O)₂R^{β}, and optionally —R^{β} is a saccharidyl group. In one embodiment, —R⁶ is selected from —OR² or —SR², and —R² is selected from —R^{α}—OR^{β} or —R^{α}—SR^{β}, and optionally —R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); and —R² is —C(O)R⁴. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β}, —R^{α}—SR^{β}, —R^{α}—S(O)R^{β} or —R^{α}—S(O)₂R^{β}; and —R^{β} is a saccharidyl group. In one embodiment, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is —C(O)R⁴; —R⁴ is selected from —R^{α}—OR^{β} or —R^{α}—SR^{β}; and —R^{β} is a saccharidyl group.

In one embodiment of the first or second aspect of the present invention, —R⁶ is selected from —OR², —SR², —S(O)R² or —S(O)₂R², and —R² is —C(O)R⁴. In one embodiment, —R⁶ is selected from —OR², —SR², —S(O)R² or —S(O)₂R², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR², —SR², —S(O)R² or —S(O)₂R², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR² or —SR², and —R² is —C(O)R⁴. In one embodiment, —R⁶ is selected from —OR² or —SR², and —R² is —C(O)R⁴, and —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —OR² or —SR², and —R² is —C(O)R⁴, and —R⁴ is selected from —R^{α}—OR^{β} or —R^{α}—SR^{β}, and —R^{β} is a saccharidyl group.

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl (preferably hydrogen or methyl); —R² is selected from —R^{β}, —R^{α}—OR^{β}, —R^{α}—SR^{β}, —R^{α}—S(O)R^{β} or —R^{α}—S(O)₂R^{β}; —R^{β} is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe. In one embodiment, —R⁶ is selected from —OR², —N(R²)(R^{2′}) or —SR²; —R^{2′} is selected from hydrogen or C₁-C₄ alkyl (preferably hydrogen or methyl); —R² is selected from —R^{β}, —R^{α}—OR^{β} or —R^{α}—SR^{β}; —R^{β} is a saccharidyl group; and —R^{α}— is selected from a C₁-C₁₂ alkylene group, wherein one, two, three or four carbon atoms in the backbone of the alkylene group may optionally be replaced by a heteroatom or group independently selected from O, S, NH or NMe.

In any of the embodiments in the five preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R⁶ is selected from —OR², —N(R²)₂, —SR², —S(O)R² or —S(O)₂R²; and —R² is selected from hydrogen, C₁-C₄ alkyl, —CO(C₁-C₄ alkyl) or —CO₂(C₁-C₄ alkyl). In one embodiment, —R⁶ is selected from —OR² or —N(R²)₂; and —R² is selected from hydrogen, C₁-C₄ alkyl, —CO(C₁-C₄ alkyl) or —CO₂(C₁-C₄ alkyl).

In one embodiment of the first or second aspect of the present invention, —R⁶ is selected from —OR², —N(R²)₂, —N(R²)(R^{2′}), —SR², —S(O)R² or —S(O)₂R²; —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe. In one embodiment, —R⁶ is selected from —OR² or —N(R²)(R^{2′}); —R^{2′} is selected from hydrogen or C₁-C₄ alkyl; —R² is selected from —R⁴, —C(O)R⁴, —C(O)—OR⁴ or —C(O)—N(R⁴)(R^{4′}); —R^{4′} is selected from hydrogen or C₁-C₄ alkyl; and —R⁴ is selected from a C₁-C₁₂ alkyl group, wherein the alkyl group may optionally be substituted with one, two, three or four halo groups, and wherein one, two, three or four carbon atoms in the backbone of the alkyl group may optionally be replaced by a heteroatom or group independently selected from O, S, NH or NMe.

In one embodiment of the first or second aspect of the present invention, —R²⁹— is selected from —N(R⁹)—, —N(OR⁹)— or —O—. In one embodiment, —R²⁹— is selected from —N(R⁹)— or —O—. In one embodiment, —R²⁹— is —N(R⁹)—.

In one embodiment of the first or second aspect of the present invention, —R⁹ is selected from —H, —R^{α}—H, —R^{β}, —R^{α}—R^{β}, —R^{α}—OR^{β}, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, —R⁹ is selected from —H, —R^{α}—H, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, —R⁹ is selected from —H, —R^{α}—H, —R^{α}—[P(R⁵)₃]Y or —R^{α}—[P(R⁵)₂(R^{5′})]. In one embodiment, —R⁹ is selected from —H, —R^{α}—H or —R^{α}—[P(R⁵)₃]Y.

In one embodiment of the first or second aspect of the present invention, each —R^{α}— is independently a C₁-C₁₂ alkylene group, a —(CH₂CH₂O)ₘ— group, a —(CH₂CH₂S)ₘ— group, a —(CH₂CH₂O)ₘ—CH₂CH₂— group or a —(CH₂CH₂S)ₘ—CH₂CH₂— group, all optionally substituted, wherein m is 1, 2, 3 or 4. In one embodiment, each —R^{α}— is independently a C₁-C₁₂ alkylene group, a —(CH₂CH₂O)ₘ— group or a —(CH₂CH₂S)ₘ— group, all optionally substituted, wherein m is 1, 2, 3 or 4. In one embodiment, each —R^{α}— is independently a C₁-C₁₂ alkylene group or a —(CH₂CH₂O)ₘ— group, both optionally substituted, wherein m is 1, 2, 3 or 4. In one embodiment, each —R^{α}— is independently an optionally substituted —(CH₂CH₂O)ₘ— group, wherein m is 1, 2, 3 or 4.

In one embodiment of the first or second aspect of the present invention, each —R^{α}— is independently a C₁-C₈ alkylene group, or a C₁-C₆ alkylene group, or a C₂-C₄ alkylene group, all optionally substituted.

In one embodiment of the first or second aspect of the present invention, each —R^{α}— is independently unsubstituted or substituted with one or more substituents independently selected from halo, C₁-C₄ alkyl, or C₁-C₄ haloalkyl. In one embodiment, each —R^{α}— is independently unsubstituted or substituted with one or two substituents independently selected from halo, C₁-C₄ alkyl, or C₁-C₄ haloalkyl. In one embodiment, each —R^{α}— is unsubstituted.

In one embodiment of the first or second aspect of the present invention, each —R^{β} is independently a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment of the first or second aspect of the present invention, at least one —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group, all optionally substituted.

In one embodiment of the first or second aspect of the present invention, at least one —R^{β} is independently a saccharidyl group. In one embodiment, each —R^{β} is independently a saccharidyl group.

In one embodiment of the first or second aspect of the present invention, each —R^{β} is independently unsubstituted or substituted with one or more substituents independently selected from halo, C₁-C₄ alkyl, or C₁-C₄ haloalkyl. In one embodiment, each —R^{β} is independently unsubstituted or substituted with one or two substituents independently selected from halo, C₁-C₄ alkyl, or C₁-C₄ haloalkyl. In one embodiment, each —R^{β} is unsubstituted.

In one embodiment of the first or second aspect of the present invention, each —R³ is independently selected from —R^{α}—H, —Re, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, at least one —R³ is independently selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, at least one —R³ is independently selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —CO₂⁻, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —CO₂⁻ and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, each —R³ is independently selected from —R^{α}—OR^{β}, —R^{α}—SR^{β}, —R^{α}—S(O)R^{β} or —R^{α}—S(O)₂R^{β}. In one embodiment, each —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R³ is independently selected from —R^{α}—OR^{β} or —R^{α}—SR^{β}. In one embodiment, each —R³ is independently selected from —R^{α}—OR^{β} or —R^{α}—SR^{β}, and —R^{β} is a saccharidyl group.

In one embodiment of the first or second aspect of the present invention, each —R⁴ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, at least one —R⁴ is independently selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]. In one embodiment, at least one —R⁴ is independently selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —CO₂, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —CO₂⁻ and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide or hexafluorophosphate).

In one embodiment of the first or second aspect of the present invention, each —R⁴ is independently selected from —R^{α}—OR^{β}, —R^{α}—SR^{β}, —R^{α}—S(O)R^{β} or —R^{α}—S(O)₂R^{β}. In one embodiment, each —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R⁴ is independently selected from —R^{α}—OR^{β} or —R^{α}—SR^{β}. In one embodiment, each —R⁴ is independently selected from —R^{α}—OR^{β} or —R^{α}—SR^{β}, and —R^{β} is a saccharidyl group.

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, at least one of —R², —R³ or —R⁴ is independently selected from —R^{α}—OR^{β} or —R^{α}—SR^{β}, and —R^{β} is a saccharidyl group.

For the purposes of the present invention, a “saccharidyl group” is any group comprising at least one monosaccharide subunit, wherein each monosaccharide subunit may optionally be substituted and/or modified. Typically, a saccharidyl group consist of one or more monosaccharide subunits, wherein each monosaccharide subunit may optionally be substituted and/or modified.

Typically, a carbon atom of a single monosaccharide subunit of each saccharidyl group is directly attached to the remainder of the compound, most typically via a single bond.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

Typically, each saccharidyl group is derived from the corresponding saccharide by substitution of a hydroxyl group of the saccharide with the group defined by the remainder of the compound.

A single bond between an anomeric carbon of a monosaccharide subunit and a substituent is called a glycosidic bond. A glycosidic group is linked to the anomeric carbon of a monosaccharide subunit by a glycosidic bond. The bond between the saccharidyl group and the remainder of the compound may be a glycosidic or a non-glycosidic bond. Typically, the bond between the saccharidyl group and the remainder of the compound is a glycosidic bond, such that the saccharidyl group is a glycosyl group. Where the bond between the saccharidyl group and the remainder of the compound is a glycosidic bond, the glycosidic bond may be in the a or p configuration. Typically, such a glycosidic bond is in the p configuration.

For the purposes of the present invention, where a saccharidyl group “contains x monosaccharide subunits”, this means that the saccharidyl group has x monosaccharide subunits and no more. In contrast, where a saccharidyl group “comprises x monosaccharide subunits”, this means that the saccharidyl group has x or more monosaccharide subunits.

Each saccharidyl group may be independently selected from a monosaccharidyl, disaccharidyl, oligosaccharidyl or polysaccharidyl group. As will be understood, a monosaccharidyl group contains a single monosaccharide subunit. Similarly, a disaccharidyl group contains two monosaccharide subunits. As used herein, an “oligosaccharidyl group” contains from 2 to 9 monosaccharide subunits. Examples of oligosaccharidyl groups include trisaccharidyl, tetrasaccharidyl, pentasaccharidyl, hexasaccharidyl, heptasaccharidyl, octasaccharidyl and nonasaccharidyl groups. As used herein, a “polysaccharidyl group” contains 10 or more monosaccharide subunits (such as 10-50, or 10-30, or 10-20, or 10-15 monosaccharide subunits).

Each monosaccharide subunit within a disaccharidyl, oligosaccharidyl or polysaccharidyl group may be the same or different. Each monosaccharide subunit within a disaccharidyl, oligosaccharidyl or polysaccharidyl group may be connected to another monosaccharide subunit within the group via a glycosidic or a non-glycosidic bond. Typically each monosaccharide subunit within a disaccharidyl, oligosaccharidyl or polysaccharidyl group is connected to another monosaccharide subunit within the group via a glycosidic bond, which may be in the a or B configuration.

Each oligosaccharidyl or polysaccharidyl group may be a linear, branched or macrocyclic oligosaccharidyl or polysaccharidyl group. Typically, each oligosaccharidyl or polysaccharidyl group is a linear or branched oligosaccharidyl or polysaccharidyl group.

In one embodiment, at least one —R^{β} is a monosaccharidyl or disaccharidyl group.

In a further embodiment, at least one —R^{β} is a monosaccharidyl group. For example, at least one —R^{β} may be a glycosyl group containing a single monosaccharide subunit, wherein the monosaccharide subunit may optionally be substituted and/or modified. Typically at least one —R^{β} is a glycosyl group containing a single monosaccharide subunit, wherein the monosaccharide subunit may optionally be substituted. More typically, at least one —R^{β} is a glycosyl group containing a single monosaccharide subunit, wherein the monosaccharide subunit is unsubstituted.

In one embodiment, at least one —R^{β} is an aldosyl group, wherein the aldosyl group may optionally be substituted and/or modified. For example, at least one —R^{β} may be selected from a glycerosyl, aldotetrosyl (such as erythrosyl or threosyl), aldopentosyl (such as ribosyl, arabinosyl, xylosyl or lyxosyl) or aldohexosyl (such as allosyl, altrosyl, glucosyl, mannosyl, gulosyl, idosyl, galactosyl or talosyl) group, any of which may optionally be substituted and/or modified.

In another embodiment, at least one —R^{β} is a ketosyl group, wherein the ketosyl group may optionally be substituted and/or modified. For example, at least one —R^{β} may be selected from an erythrulosyl, ketopentosyl (such as ribulosyl or xylulosyl) or ketohexosyl (such as psicosyl, fructosyl, sorbosyl or tagatosyl) group, any of which may optionally be substituted and/or modified.

Each monosaccharide subunit may be present in a ring-closed (cyclic) or open-chain (acyclic) form. Typically, each monosaccharide subunit in at least one —R^{β} is present in a ring-closed (cyclic) form. For example, at least one —R^{β} may be a glycosyl group containing a single ring-closed monosaccharide subunit, wherein the monosaccharide subunit may optionally be substituted and/or modified. Typically in such a scenario, at least one —R^{β} is a pyranosyl or furanosyl group, such as an aldopyranosyl, aldofuranosyl, ketopyranosyl or ketofuranosyl group, any of which may optionally be substituted and/or modified. More typically, at least one —R^{β} is a pyranosyl group, such as an aldopyranosyl or ketopyranosyl group, any of which may optionally be substituted and/or modified.

In one embodiment, at least one —R^{β} is selected from a ribopyranosyl, arabinopyranosyl, xylopyranosyl, lyxopyranosyl, allopyranosyl, altropyranosyl, glucopyranosyl, mannopyranosyl, gulopyranosyl, idopyranosyl, galactopyranosyl or talopyranosyl group, any of which may optionally be substituted and/or modified.

In a further embodiment, at least one —R^{β} is a glucosyl group, such as a glucopyranosyl group, wherein the glucosyl or the glucopyranosyl group may optionally be substituted and/or modified. Typically, at least one —R^{β} is a glucosyl group, wherein the glucosyl group is optionally substituted. More typically, at least one —R^{β} is an unsubstituted glucosyl group.

Each monosaccharide subunit may be present in the D- or L-configuration. Typically, each monosaccharide subunit is present in the configuration in which it most commonly occurs in nature.

In one embodiment, at least one —R^{β} is a D-glucosyl group, such as a D-glucopyranosyl group, wherein the D-glucosyl or the D-glucopyranosyl group may optionally be substituted and/or modified. Typically, at least one —R^{β} is a D-glucosyl group, wherein the D-glucosyl group is optionally substituted. More typically, at least one —R^{β} is an unsubstituted D-glucosyl group.

For the purposes of the present invention, in a substituted monosaccharidyl group or monosaccharide subunit:
(a) one or more of the hydroxyl groups of the monosaccharidyl group or monosaccharide subunit are each independently replaced with —H, —F, —Cl, —Br, —I, —CF₃, —CCl₃, —CBr₃, —CI₃, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃+, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(b) one, two or three hydrogen atoms directly attached to a carbon atom of the monosaccharidyl group or monosaccharide subunit are each independently replaced with —F, —Cl, —Br, —I, —CF₃, —CCl₃, —CBr₃, —CI₃, —OH, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃⁺, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(c) one or more of the hydroxyl groups of the monosaccharidyl group or monosaccharide subunit, together with the hydrogen attached to the same carbon atom as the hydroxyl group, are each independently replaced with ═O, ═S, ═NR^{b}, or ═N(R^{b})₂⁺; and/or
(d) any two hydroxyl groups of the monosaccharidyl group or monosaccharide subunit are together replaced with —O—R^{c}—, —S—R^{c}—, —SO—R^{c}—, —SO₂—R^{c}—, or —NR^{b}—R^{c}—; wherein:
each —R^{a}— is independently a substituted or unsubstituted alkylene, alkenylene or alkynylene group which optionally includes one or more heteroatoms each independently selected from O, N and S in its carbon skeleton and preferably comprises 1-10 carbon atoms;
each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms; and
each —R^{c}— is independently a chemical bond, or a substituted or unsubstituted alkylene, alkenylene or alkynylene group which optionally includes one or more heteroatoms each independently selected from O, N and S in its carbon skeleton and preferably comprises 1-10 carbon atoms;
provided that the monosaccharidyl group or monosaccharide subunit comprises at least one, preferably at least two or at least three, —OH, —O—R^{b}, —O—SO—R^{b}, —O—SO₂—R^{b}, —O—SO₂—OR^{b}, —O—SO—N(R^{b})₂, —O—SO₂—N(R^{b})₂, —OP(R^{b})₂, —OPO(R^{b})₂, —OSi(R^{b})₃, —O—CO—R^{b}, —O—CO—OR^{b}, —O—CO—N(R^{b})₂, or —O—R^{c}.

Typically, in a substituted monosaccharidyl group or monosaccharide subunit:
(a) one or more of the hydroxyl groups of the monosaccharidyl group or monosaccharide subunit are each independently replaced with —H, —F, —CF₃, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(b) one or two of the hydrogen atoms directly attached to a carbon atom of the monosaccharidyl group or monosaccharide subunit are each independently replaced with —F, —CF₃, —OH, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(c) one hydroxyl group of the monosaccharidyl group or monosaccharide subunit, together with the hydrogen attached to the same carbon atom as the hydroxyl group, is replaced with ═O; and/or
(d) any two hydroxyl groups of the monosaccharidyl group or monosaccharide subunit are together replaced with —O—R^{c}— or —NR^{b}—R^{c}—;
wherein:
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms; and
   each —R^{c}— is independently a substituted or unsubstituted alkylene, alkenylene or alkynylene group which optionally includes one, two or three heteroatoms each independently selected from 0 and N in its carbon skeleton and comprises 1-8 carbon atoms;
   provided that the monosaccharidyl group or monosaccharide subunit comprises at least two, preferably at least three, —OH, —O—R^{b}, —OPO(R^{b})₂, —OSi(R^{b})₃, —O—CO—R^{b}, —O—CO—OR^{b}, —O—CO—N(R^{b})₂, or —O—R^{c}.

In one embodiment, —R^{β} is a saccharidyl group and one or more of the hydroxyl groups of the saccharidyl group are each independently replaced with —O—CO—R^{b}, wherein each —R^{b} is independently C₁-C₄ alkyl, preferably methyl. In one embodiment, —R^{β} is a saccharidyl group and all of the hydroxyl groups of the saccharidyl group are each independently replaced with —O—CO—R^{b}, wherein each —R^{b} is independently C₁-C₄ alkyl, preferably methyl.

In a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, is partially unsaturated; and/or
(b) the ring oxygen of the modified monosaccharidyl group or monosaccharide subunit, or what would be the ring oxygen in the ring-closed form of the modified monosaccharidyl group or monosaccharide subunit, is replaced with —S— or —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms.

Alternately, where the modified monosaccharide subunit forms part of a disaccharidyl, oligosaccharidyl or polysaccharidyl group, —R^{d} may be a further monosaccharide subunit or subunits forming part of the disaccharidyl, oligosaccharidyl or polysaccharidyl group, wherein any such further monosaccharide subunit or subunits may optionally be substituted and/or modified.

Typically, in a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, contains a single C═C; and/or
(b) the ring oxygen of the modified monosaccharidyl group or monosaccharide subunit, or what would be the ring oxygen in the ring-closed form of the modified monosaccharidyl group or monosaccharide subunit, is replaced with —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms.

Typical examples of substituted and/or modified monosaccharide subunits include those corresponding to:
(i) deoxy sugars, such as deoxyribose, fucose, fuculose and rhamnose, wherein a hydroxyl group of the monosaccharidyl group or monosaccharide subunit has been replaced by —H;
(ii) amino sugars, such as glucosamine and galactosamine, wherein a hydroxyl group of the monosaccharidyl group or monosaccharide subunit has been replaced by —NH₂, most typically at the 2-position; and
(iii) sugar acids, containing a —COOH group, such as aldonic acids (e.g. gluconic acid), ulosonic acids, uronic acids (e.g. glucuronic acid) and aldaric acids (e.g. gularic or galactaric acid).

In one embodiment of the first or second aspect of the present invention, at least one —R^{β} is a monosaccharidyl group selected from:

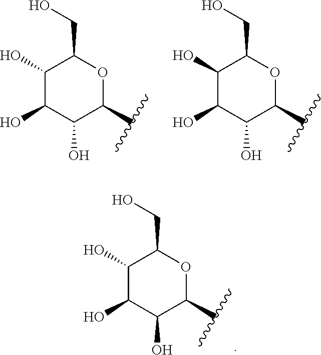 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]

In one embodiment of the first or second aspect of the present invention, at least one —R^{β} is:

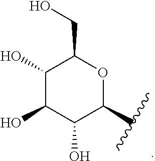 [SMILES: *[C@@H]1O[C@H](CO)[C@@H](O)[C@H](O)[C@H]1O]

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β} (preferably from —R^{α}—OR^{β} or —R^{α}—SR^{β}), and —R^{β} is selected from:

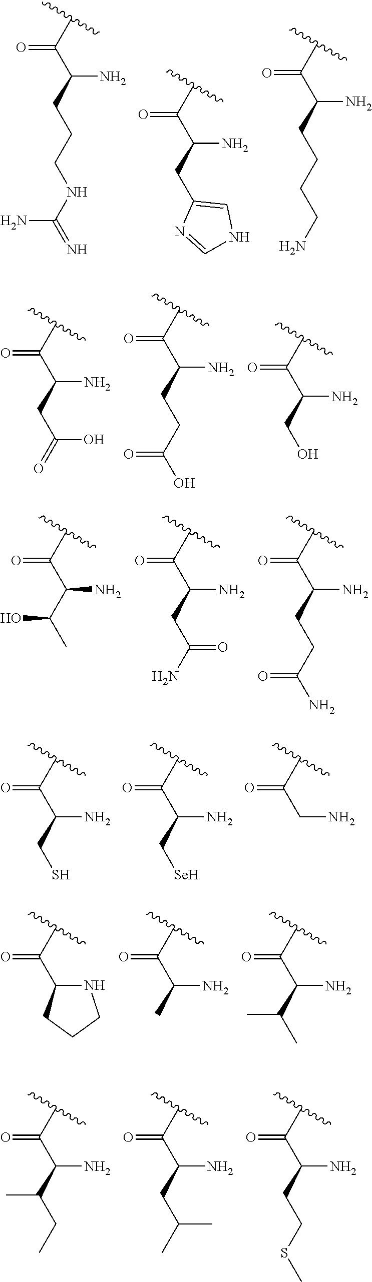

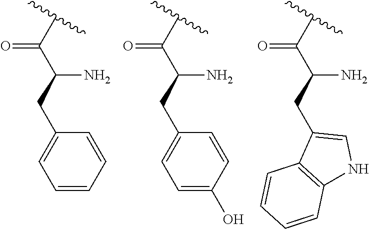

In one embodiment of the first or second aspect of the present invention, at least one of —R², —R³ or —R⁴ is independently selected from —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})], or —R^{α}—[R^{7′}], preferably at least one of —R², —R³ or —R⁴ is independently selected from —R^{α}—[P(R⁵)₃]Y or —R^{α}—[P(R⁵)₂(R^{5′})]. In one embodiment, at least one of —R², —R³ or —R⁴ is independently selected from —R^{α}—[N(R⁵)₃]Y, —R^{α}—[P(R⁵)₃]Y, or —R^{α}—[R⁷]Y, preferably at least one of —R², —R³ or —R⁴ is —R^{α}—[P(R⁵)₃]Y. In one embodiment, at least one of —R², —R³ or —R⁴ is independently selected from:

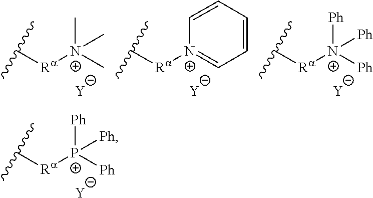

preferably at least one of —R², —R³ or —R⁴ is —R^{α}—[PPh₃]Y.

In the first or second aspect of the present invention, each —R⁵ may be the same or different. In a preferred embodiment, each —R⁵ is the same.

In one embodiment of the first or second aspect of the present invention, each —R⁵ is independently unsubstituted or substituted with one or two substituents. In one embodiment, each —R⁵ is unsubstituted.

In one embodiment of the first or second aspect of the present invention, —R⁷ is unsubstituted or substituted with one or two substituents. In one embodiment, —R⁷ is unsubstituted.

In one embodiment of the first or second aspect of the present invention, —R⁷ is not substituted at the 4-position of the pyridine ring with a halo group. In one embodiment, —R⁷ is unsubstituted at the 4-position of the pyridine ring. In one embodiment, —R⁷ is unsubstituted.

In one embodiment of the first or second aspect of the present invention, each of —R¹, —R⁶, —R⁹, —R²⁶ and —R²⁹ independently comprises from 1 to 100 atoms other than hydrogen, preferably from 1 to 80 atoms other than hydrogen, preferably from 1 to 60 atoms other than hydrogen, preferably from 1 to 50 atoms other than hydrogen, and preferably from 1 to 45 atoms other than hydrogen.

In a particularly preferred embodiment, the first or second aspect of the present invention provides a compound of formula (I) or a complex of formula (II):

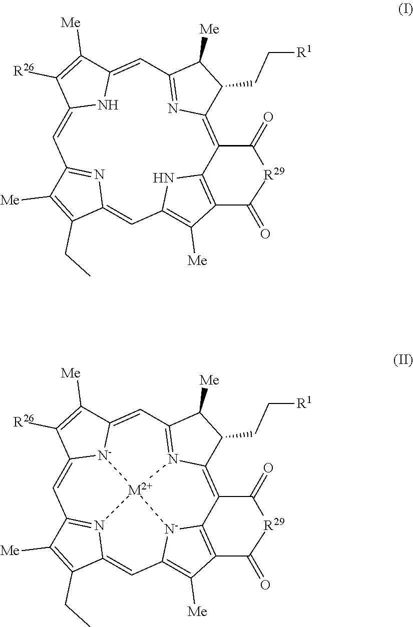

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SRH, —R^{α}—S(O)R^{β}, —R^{α}—S(O)₂R^{β}, —R^{α}—NH₂, —R^{α}—NH(R), —R^{α}—N(R^{β})₂, —R^{α}—X, —[(CH₂)PQ]ᵣ—(CH₂)ₛ—[N(R⁵)₃]Y, —[(CH₂)PQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups; —R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ (CH₂)ₛ—[R^{7′}];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
Q is O, S, NH or NMe [preferably Q is O];
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
M²⁺ is a metal cation [preferably M²⁺ is a metal cation selected from Zn²⁺, Cu²⁺, Fe²⁺, Pd²⁺ or Pt²⁺];
n is 1, 2, 3, 4, 5 or 6;
p is 0, 1, 2, 3 or 4;
r is 0, 1, 2, 3, 4, 5 or 6; and
S is 0, 1, 2, 3, 4, 5, 6, 7, 8, 9, 10, 11 or 12;
provided that at least one of —R², —R³, —R⁴ and —R⁹ is selected from —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[P(R⁵)₂(R^{5′})] or —[(CH₂)ₚQ]ᵣ—(CH₂)ₛ—[R^{7′}].

In a particularly preferred embodiment, the first or second aspect of the present invention provides a compound of formula (I) or a complex of formula (2):

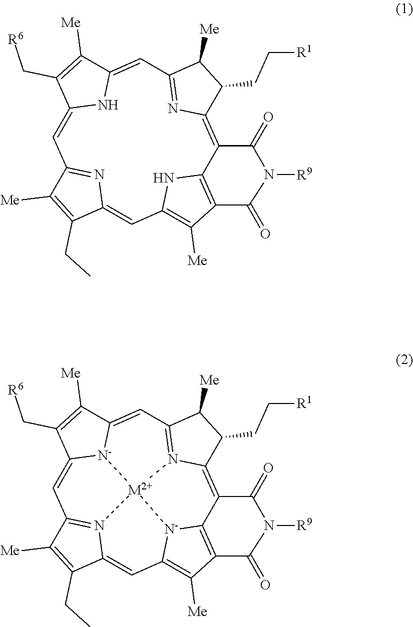

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{c}—X, —[(CH₂)ₚQ]ᵣ—(CH₂)ₛ—[N(R⁵)₃]Y, —[(CH₂)ₚQ]ᵣ (CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}]; —R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R_{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ (CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ (CH₂)ₛ—[R^{7′}];
Q is O, S, NH or NMe [preferably Q is O];
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
M²⁺ is a metal cation [preferably M²⁺ is a metal cation selected from Zn²⁺, Cu²⁺, Fe²⁺, Pd²⁺ or Pt²⁺];
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12;
provided that at least one of —R², —R³, —R⁴ and —R⁹ is selected from —[(CH₂)ₚQ]ᵣ (CH₂)ₛ—[P(R⁵)₃]Y, —[(CH₂)ₚQ]ᵣ (CH₂)ₛ—[R⁷]Y, —[(CH₂)ₚQ]ᵣ—(CH₂)ₛ—[P(R⁵)₂(R^{5′})] or —[(CH₂)ₚQ]ᵣ—(CH₂)ₛ—[R^{7′}].

In a particularly preferred embodiment, the first or second aspect of the present invention provides a compound of formula (I) or a complex of formula (II):

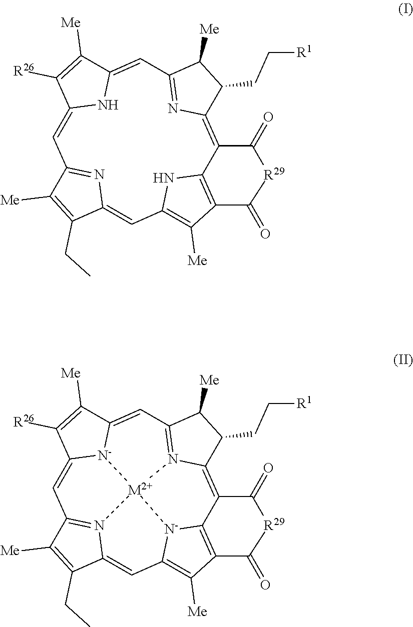

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR^{3′} or —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R^{3′} and —R^{4′}, each independently, is selected from hydrogen or C₁-C₃ alkyl [preferably —R^{3′} and —R^{4′}, each independently, is selected from hydrogen or methyl];
—R^{α}—, each independently, is selected from a C₁-C₄₂ alkylene group [preferably a C₁-C₂₂ alkylene group], wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂⁻ or phenyl substituted with —CO₂⁻, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —OR², —N(R²)₂, —SR², —S(O)R², —S(O)₂R², or —X;
—R⁷ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{7′} is —[NC₅H₅] substituted with —CO₂⁻ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ (CH₂)ₛ—[R^{7′}];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
Q is O, S, NH or NMe [preferably Q is O];
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
M²⁺ is a metal cation [preferably M²⁺ is a metal cation selected from Zn²⁺, Cu²⁺, Fe²⁺, Pd²⁺ or Pt²⁺];
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12;
provided that at least one of —R², —R³, —R⁴ and —R⁹ is selected from —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[P(R⁵)₂(R^{5′})] or —[(CH₂)ₚQ]ᵣ—(CH₂)ₛ—[R^{7′}].

In a particularly preferred embodiment, the first or second aspect of the present invention provides a compound of formula (I) or a complex of formula (II):

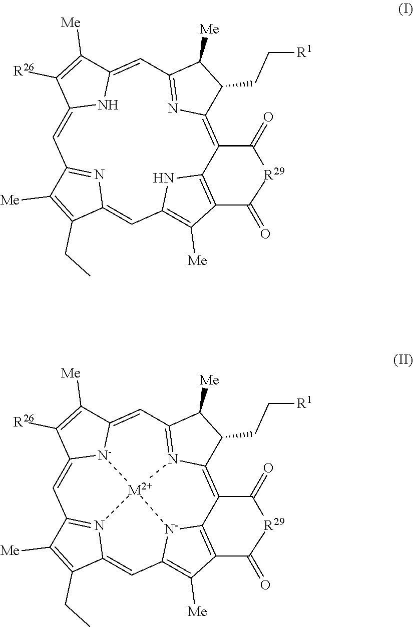

or a pharmaceutically acceptable salt thereof, wherein:
—R¹ is selected from —C(O)—OR^{3′} or —C(O)—N(R³)(R^{3′});
—R², each independently, is selected from —H, —C(O)R⁴, —C(O)—OR⁴, —C(O)—SR⁴, —C(O)—N(R⁴)(R^{4′}), —R^{α}—H, —[(CH₂)ₚQ]ᵣ—(CH₂)ₛ—[P(R⁵)₃]Y or —[(CH₂)ₚQ]ᵣ—(CH₂)ₛ—[P(R⁵)₂(R^{5′})];
—R³ and —R⁴, each independently, is selected from —H, —R^{α}—H, —[(CH₂)ₚQ]ᵣ—(CH₂)ₛ—[P(R⁵)₃]Y or —[(CH₂)ₚQ]ᵣ—(CH₂)ₛ—[P(R⁵)₂(R^{5′})];
—R^{3′} and —R^{4′}, each independently, is selected from hydrogen or methyl;
—R^{α}—, each independently, is selected from a C₁-C₂₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂⁻ or phenyl substituted with —CO₂⁻, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —OR², —N(R²)₂, —SR², —S(O)R² or —S(O)₂R²;
—R⁹ is selected from —H, —C(O)R⁴, —C(O)—OR⁴, —C(O)—SR⁴, —C(O)—N(R⁴)(R^{4′}), —R^{α}—H, —[(CH₂)ₚQ]ᵣ—(CH₂)ₛ—[P(R⁵)₃]Y or —[(CH₂)ₚQ]ᵣ—(CH₂)ₛ—[P(R⁵)₂(R^{5′})];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
Q is O, S, NH or NMe;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
M²⁺ is a metal cation [preferably M²⁺ is a metal cation selected from Zn²⁺, Cu²⁺, Fe²⁺, Pd²⁺ or Pt²⁺];
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12;
provided that at least one of —R², —R³, —R⁴ and —R⁹ is selected from —[(CH₂)ₚQ]ᵣ—(CH₂)ₛ—[P(R⁵)₃]Y or —[(CH₂)ₚQ]ᵣ—(CH₂)ₛ—[P(R⁵)₂(R^{5′})].

In a particularly preferred embodiment, the first or second aspect of the present invention provides a compound of formula (I) or a complex of formula (II):

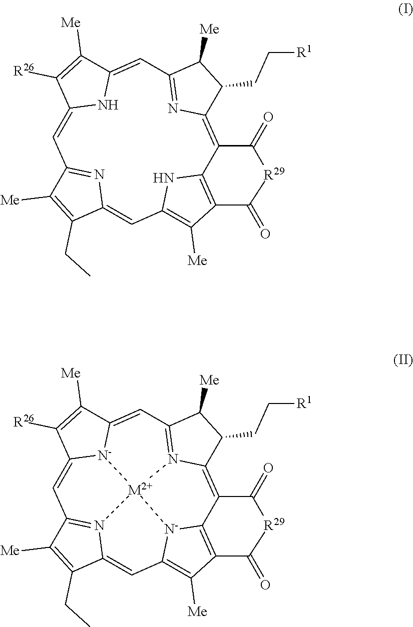

or a pharmaceutically acceptable salt thereof, wherein:
—R¹ is selected from —C(O)—OR^{3′} or —C(O)—N(R³)(R^{3′});
—R², each independently, is selected from —H, —C(O)R⁴, —C(O)—OR⁴, —C(O)—SR⁴, —C(O)—N(R⁴)(R^{4′}), —R^{α}—H or —[(CH₂)ₚQ]ᵣ—(CH₂)ₛ—[P(R⁵)₃]Y;
—R³ and —R⁴, each independently, is selected from —H, —R^{α}—H or —[(CH₂)ₚQ]ᵣ (CH₂)ₛ—[P(R⁵)₃]Y;
—R^{3′} and —R^{4′}, each independently, is selected from hydrogen or methyl;
—R^{α}—, each independently, is selected from a C₁-C₂₂ alkylene group, wherein one or more (such as one, two, three, four, five, six, seven or eight) carbon atoms in the backbone of the alkylene group may optionally be replaced by an oxygen atom;
—R⁵, each independently, is selected from phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —OR² or —N(R²)₂;
—R⁹ is selected from —H, —C(O)R⁴, —C(O)—OR⁴, —C(O)—SR⁴, —C(O)—N(R⁴)(R^{4′}), —R^{α}—H or —[(CH₂)ₚQ]ᵣ—(CH₂)ₛ—[P(R⁵)₃]Y;
—R²⁶ is selected from —CH₂R⁶, —CH₂CH₃ or —CH═CH₂;
—R²⁹— is selected from —N(R⁹)—, —N(OR⁹)— or —O—;
Q is O;
Y is a counter anion selected from halide or hexafluorophosphate;
Z is a counter cation selected from Li⁺, Na⁺, K⁺, Mg²⁺, Ca²⁺ or NH₄⁺;
M²⁺ is a metal cation selected from Zn²⁺, Cu²⁺, Fe²⁺, Pd²⁺ or Pt²⁺;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12;
provided that at least one of —R², —R³, —R⁴ and —R⁹ is —[(CH₂)ₚQ]ᵣ (CH₂)ₛ—[P(R⁵)₃]Y.

In a particularly preferred embodiment, the first or second aspect of the present invention provides a compound of formula (I) or a complex of formula (2):

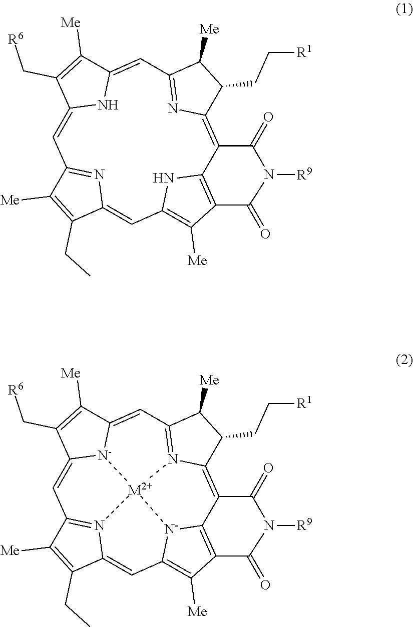

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR^{3′} or —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ (CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
—R^{3′} and —R^{4′}, each independently, is selected from hydrogen or C₁-C₃ alkyl [preferably —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂⁻ or phenyl substituted with —CO₂⁻, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{7′} is —[NC₅H₅] substituted with —CO₂⁻ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ (CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁷]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{7′}];
Q is O, S, NH or NMe [preferably Q is O];
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
M²⁺ is a metal cation [preferably M²⁺ is a metal cation selected from Zn²⁺, Cu²⁺, Fe²⁺, Pd²⁺ or Pt²⁺];
n is 1, 2, 3, 4, 5 or 6;
p is 0, 1, 2, 3 or 4;
r is 0, 1, 2, 3, 4, 5 or 6; and
S is 0, 1, 2, 3, 4, 5, 6, 7, 8, 9, 10, 11 or 12;
provided that at least one of —R², —R³, —R⁴ and —R⁹ is selected from —[(CH₂)ₚQ]ᵣ (CH₂)ₛ—[P(R⁵)₃]Y, —[(CH₂)ₚQ]ᵣ (CH₂)ₛ—[R⁷]Y, —[(CH₂)ₚQ]ᵣ—(CH₂)ₛ—[P(R⁵)₂(R^{5′})] or —[(CH₂)ₚQ]ᵣ—(CH₂)ₛ—[R^{7′}].

In these six preferred embodiments of the preceding paragraphs, each —R⁵ may be the same or different; preferably each —R⁵ is the same.

In another preferred embodiment of the first or second aspect of the present invention, the compound is a compound of formula (Ia), (Ib), (Ic), (Id), (Ie), (If), (Ig), (Ih), (Ii), (Ij), (Ik) or (Im) [preferably the compound is a compound of formula (Ib), (If) or (Ij)]:

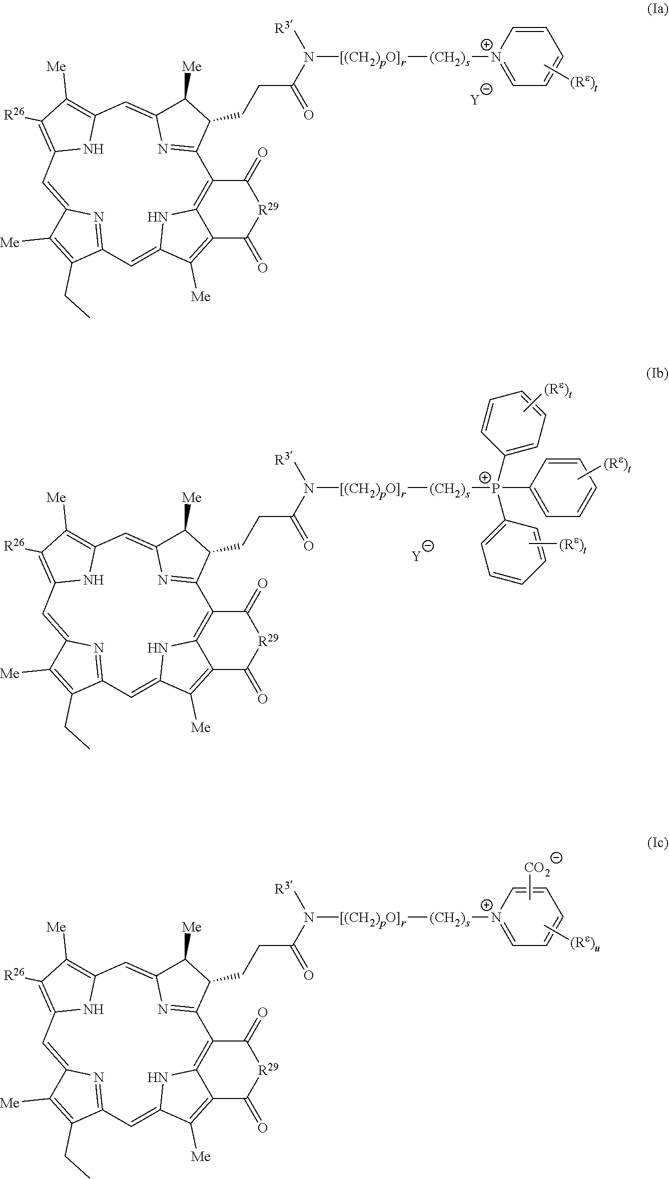

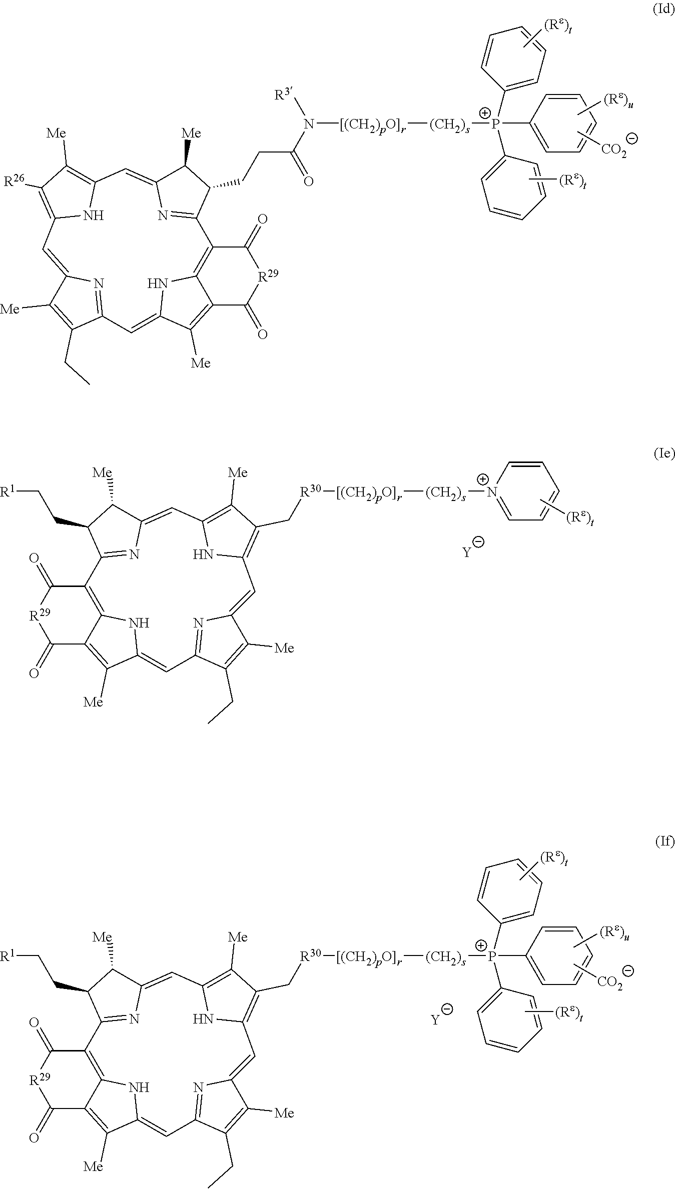

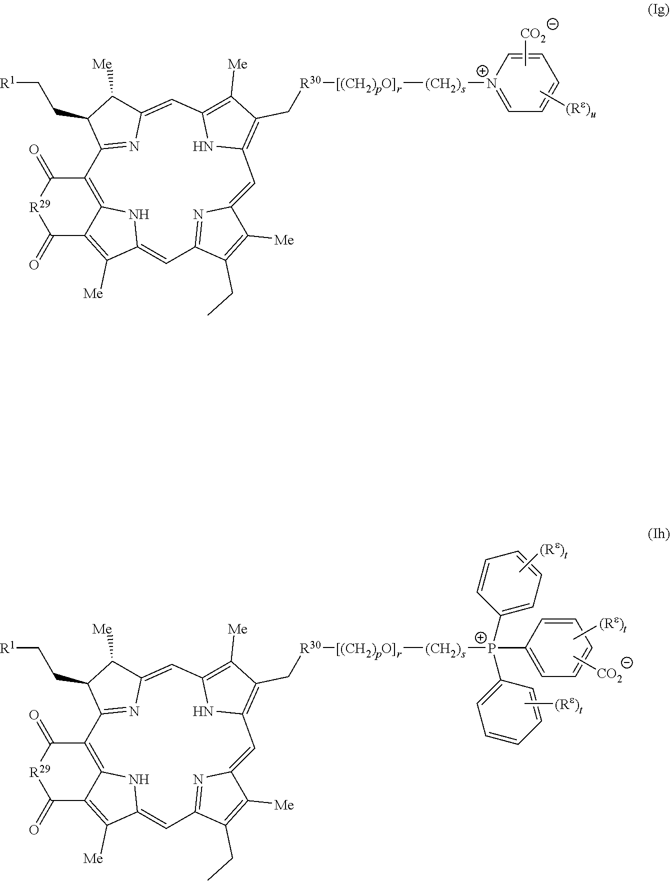

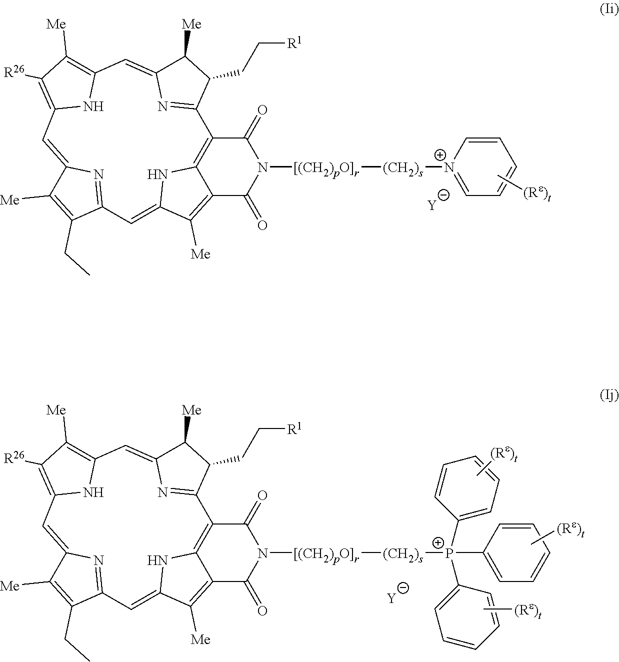

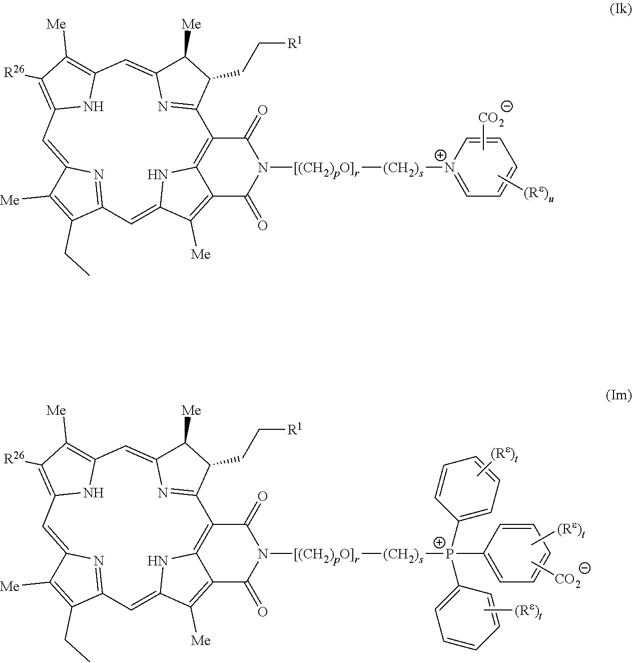

or a metal cation complex thereof, or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR^{3′} or —C(O)—N(R³)(R^{3′})];
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, or —R^{α}—X;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, or —R^{α}—X;
—R^{3′} and —R^{4′}, each independently, is selected from hydrogen or C₁-C₃ alkyl [preferably —R^{3′} and —R^{4′}, each independently, is selected from hydrogen or methyl];
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚO]ᵣ (CH₂)ₛ—[N(R⁵)₃]Y, —[(CH₂)ₚO]ᵣ—(CH₂)ₛ—[P(R⁵)₃]Y, —[(CH₂)ₚO]ᵣ—(CH₂)ₛ—[R⁷]Y, —[(CH₂)ₚO]ᵣ—(CH₂)ₛ—[N(R⁵)₂(R^{5′})], —[(CH₂)ₚO]ᵣ—(CH₂)ₛ—[P(R⁵)₂(R^{5′})] or —[(CH₂)ₚO]ᵣ (CH₂)ₛ—[R^{7′}];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
—R³⁰— is selected from —O—, —O(CO)—, —O(CO)NH—, —O(CO)NMe-, —NH—, —NH(CO)—, —NMe-, —NMe(CO)—, —N(CO₂Me)- or —N(CO₂Et)-;
—R^{α}—, each independently, is selected from a C₁-C₂₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five or six) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

In another preferred embodiment of the first or second aspect of the present invention, the compound is a compound of formula (IA), (IB), (IC), (ID), (IE), (IF), (IG) or (IH):

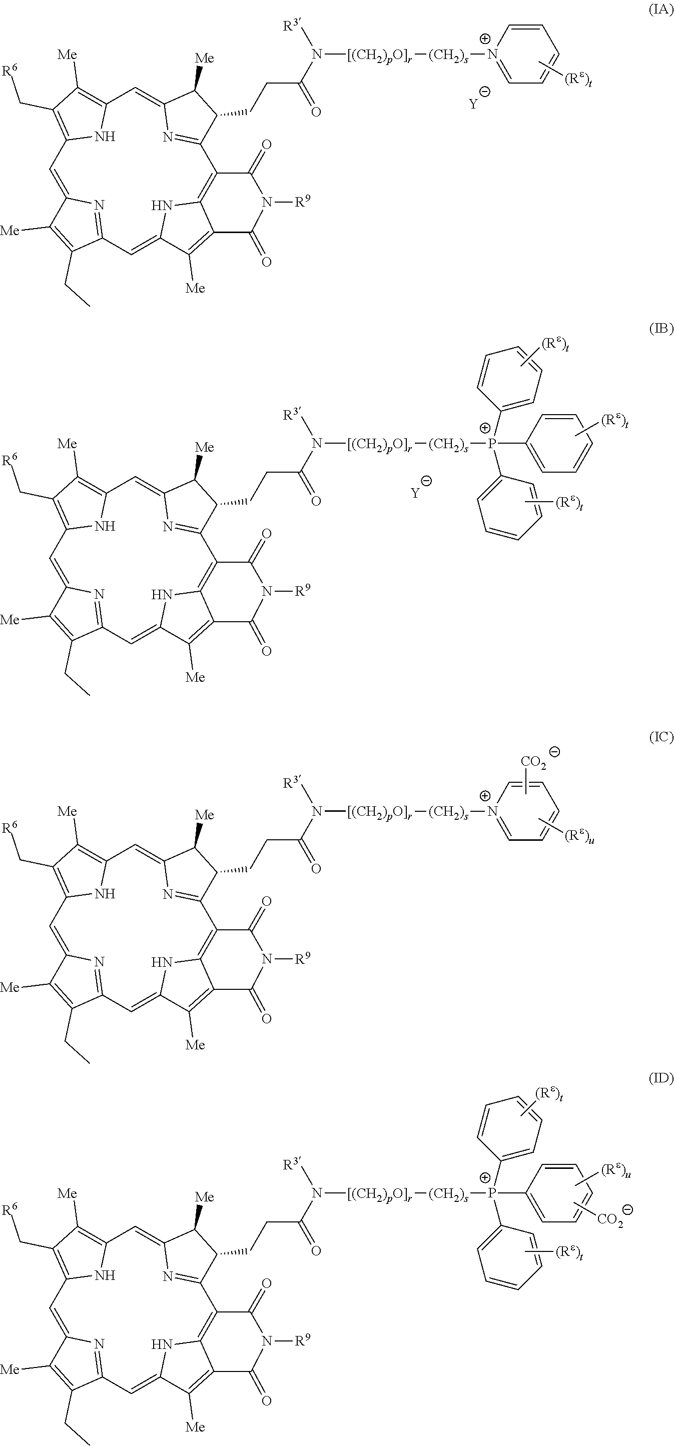

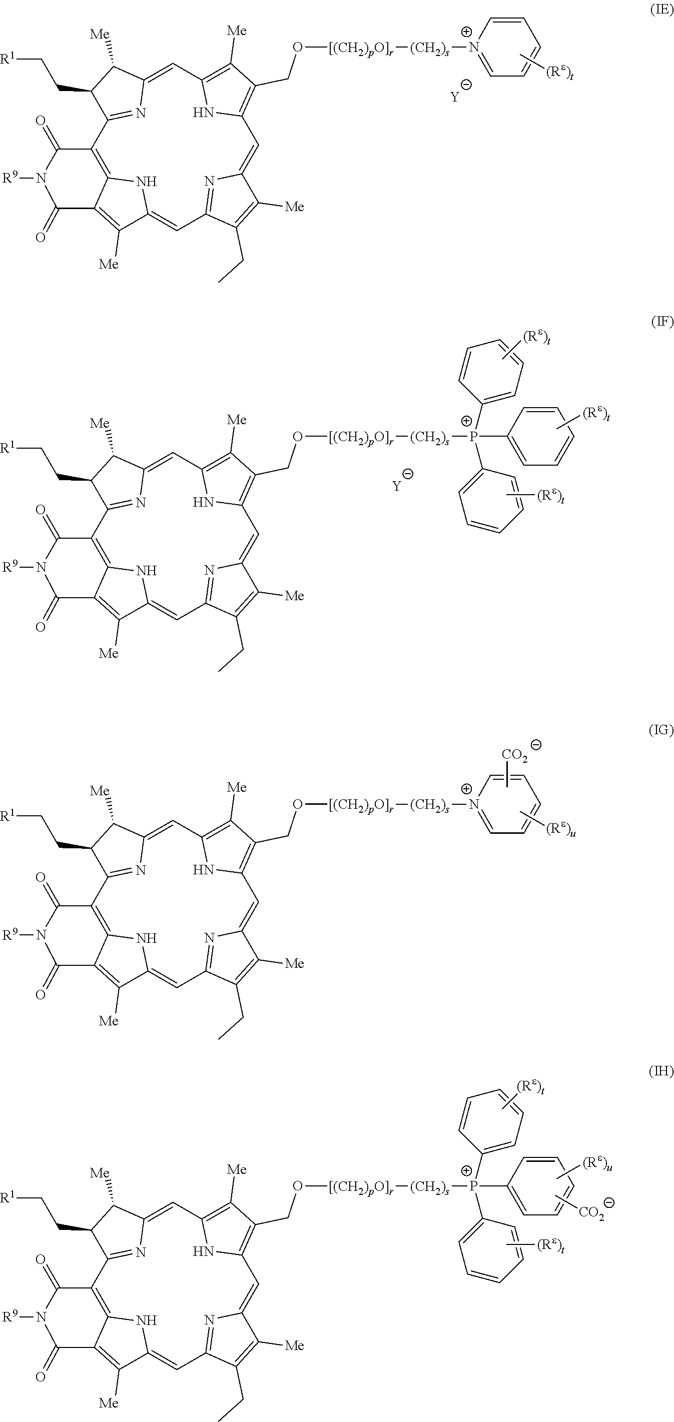

or a metal cation complex thereof, or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′});
—R², each independently, is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R^{3′} and —R^{4′}, each independently, is selected from hydrogen or C₁-C₃ alkyl [preferably —R^{3′} and —R^{4′}, each independently, is selected from hydrogen or methyl];
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X [preferably —R⁶ is selected from —OR², —SR², —S(O)R², —S(O)₂R², or —X];
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{α}—X, —[(CH₂)ₚO]ᵣ (CH₂)ₛ—[N(R⁵)₃]Y, —[(CH₂)ₚO]ᵣ—(CH₂)ₛ—[P(R⁵)₃]Y, —[(CH₂)ₚO]ᵣ—(CH₂)ₛ—[R⁷]Y, —[(CH₂)ₚO]ᵣ—(CH₂)ₛ—[N(R⁵)₂(R^{5′})], —[(CH₂)ₚO]ᵣ—(CH₂)ₛ—[P(R⁵)₂(R^{5′})] or —[(CH₂)ₚO]ᵣ (CH₂)ₛ—[R^{7′}];
—R^{α}—, each independently, is selected from a C₁-C₁₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five or six) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺];
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (Ia), (Ib), (Ic), (Id), (Ie), (If), (Ig), (Ih), (Ii), (Ij), (Ik), (Im), (IA), (IB), (IC), (ID), (IE), (IF), (IG), (IH) and complexes and salts thereof according to the first and second aspect of the present invention comprise a moiety —[(CH₂)ₚO]ᵣ—(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ(CH₂)ₛ— is —(CH₂)₁₋₁₂—.

In another preferred embodiment, the first or second aspect of the present invention provides a compound of formula (I′) or a complex of formula (II′):

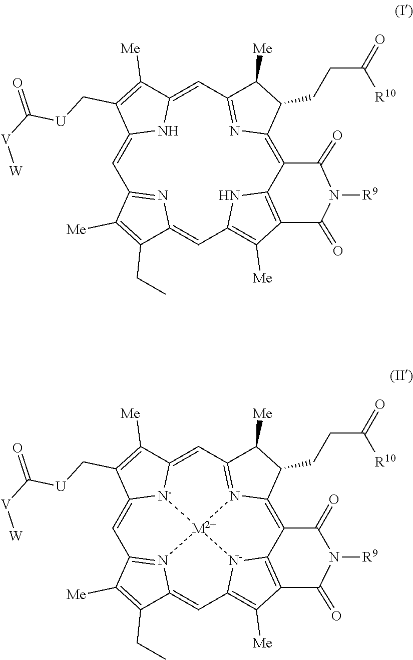

or a pharmaceutically acceptable salt thereof, wherein:
—U— is —O—, —N(R^{u})— or —S—;
—V— is —CH₂—, —O—, —N(R^{v})— or —S—;
—W— is —R^{α}—[P(R⁵)₃]Y, —R^{α}—[R⁷]Y, —R^{α}—[P(R⁵)₂(R^{5′})] or —R^{α}—[R^{7′}];
—R⁹ is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{α}—OH, —R^{α}—SH, —R^{α}—NH₂, —R^{α}—[P(R⁵)₃]Y, —R^{α}—[R⁷]Y, —R^{α}—[P(R⁵)₂(R^{5′})] or —R^{c}—[R^{7′}];
—R¹⁰ is selected from —OH or —O—(C₁-C₄ alkyl);
—R^{α}— is selected from a C₁-C₁₂ alkylene group, wherein the alkylene group may optionally be substituted with one or more (such as one, two, three or four) C₁-C₄ alkyl, C₁-C₄ haloalkyl or halo groups, and wherein one or more (such as one, two, three or four) carbon atoms in the backbone of the alkylene group may optionally be replaced by a heteroatom or group independently selected from O, S, NH or NMe;
—R⁴, each independently, is selected from hydrogen or C₁-C₄ alkyl;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{u} is hydrogen or C₁-C₄ alkyl;
—R^{v} is hydrogen or C₁-C₄ alkyl;
n is 1, 2, 3, 4, 5 or 6;
Y is a counter anion [preferably Y is a counter anion selected from halide or hexafluorophosphate];
Z is a counter cation [preferably Z is a counter cation selected from Li⁺, Na⁺, K⁺, Mg²⁺, Ca²⁺ or NH₄⁺]; and
M²⁺ is a metal cation [preferably M²⁺ is a metal cation selected from Zn²⁺, Cu²⁺, Fe²⁺, Pd²⁺ or Pt²⁺].

In another preferred embodiment, the first or second aspect of the present invention provides a compound of formula (I′) or a complex of formula (II′):

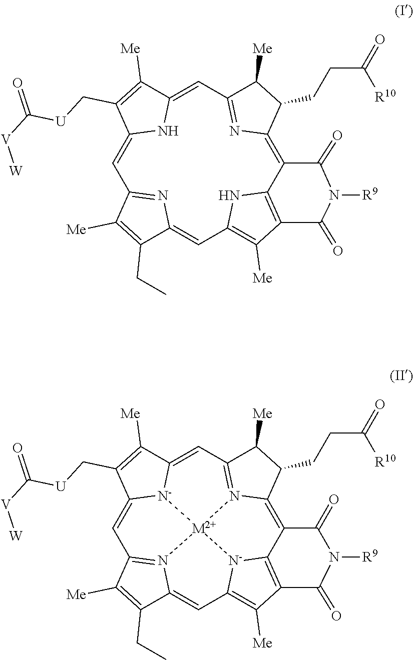

or a pharmaceutically acceptable salt thereof, wherein:
—U— is —O—, —N(R^{u})— or —S—;
—V— is —CH₂—, —O—, —N(R^{v})— or —S—;
—W— is —R^{α}—[P(R⁵)₃]Y or —R^{α}—[R⁷]Y;
—R⁹ is selected from —H, —R^{α}—H, —R^{α}—[P(R⁵)₃]Y or —R^{α}—[R⁷]Y;
—R¹⁰ is selected from —OH or —O—(C₁-C₄ alkyl);
—R^{α}— is selected from a C₁-C₁₂ alkylene group (preferably a C₁-C₉ alkylene group, preferably a C₂-C₆ alkylene group), wherein one or more (such as one, two, three or four, preferably one or two) carbon atoms in the backbone of the alkylene group may optionally be replaced by a heteroatom or group independently selected from O, S, NH or NMe (preferably O, NH or NMe, preferably O);
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl) or —O(C₁-C₄ haloalkyl);
—R⁷ is —[NC₅H₅] optionally substituted with one or more C₁-C₄ alkyl, C₁-C₄ haloalkyl, —O(C₁-C₄ alkyl) or —O(C₁-C₄ haloalkyl);
—R^{u} is hydrogen or C₁-C₄ alkyl;
—R^{v} is hydrogen or C₁-C₄ alkyl;
n is 1, 2, 3, 4, 5 or 6;
Y is a counter anion [preferably Y is a counter anion selected from halide or hexafluorophosphate]; and
M²⁺ is a metal cation [preferably M²⁺ is a metal cation selected from Zn²⁺, Cu²⁺, Fe²⁺, Pd²⁺ or Pt²⁺].

In another preferred embodiment, the first or second aspect of the present invention provides a compound of formula (I″) or a complex of formula (II″):

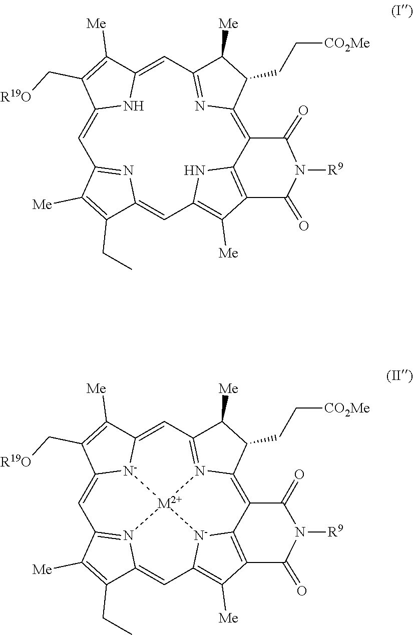

or a pharmaceutically acceptable salt thereof, wherein:
—R⁹ is selected from —H, —R^{α}—H, —R^{α}—[P(R⁵)₃]Y or —R^{α}—[R⁷]Y;
—R¹⁹ is selected from —H, —R^{α}—H, —R^{α}—[P(R⁵)₃]Y or —R^{α}—[R⁷]Y;
wherein at least one of —R⁹ and —R¹⁹ is selected from —R^{α}—[P(R⁵)₃]Y or —R^{α}—[R⁷]Y;
—R^{α}— is selected from a C₁-C₁₂ alkylene group (preferably a C₁-C₉ alkylene group, preferably a C₂-C₆ alkylene group), wherein one or more (such as one, two, three or four, preferably one or two) carbon atoms in the backbone of the alkylene group may optionally be replaced by a heteroatom or group independently selected from O, S, NH or NMe (preferably O, NH or NMe, preferably O);
—R⁵, each independently, is selected from C₁-C₃ alkyl or phenyl;
—R⁷ is —[NC₅H₅];
Y is a counter anion [preferably Y is a counter anion selected from halide or hexafluorophosphate]; and
M²⁺ is a metal cation [preferably M²⁺ is a metal cation selected from Zn²⁺, Cu²⁺, Fe²⁺, Pd²⁺ or Pt²⁺].

Preferably in the compound or complex according to the first or second aspect of the present invention, the compound or complex is:

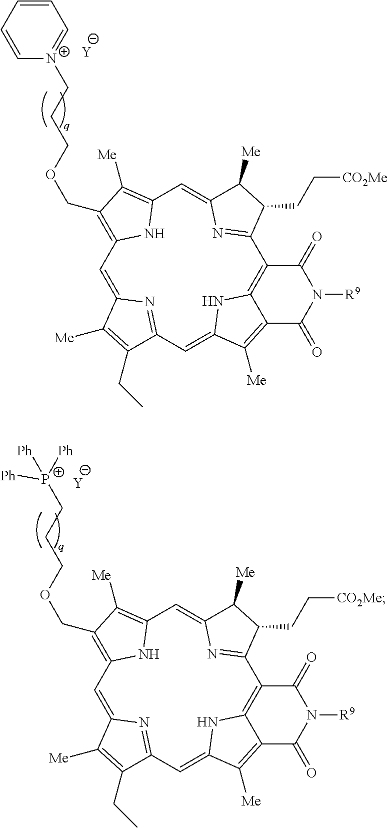

or a metal cation complex thereof, or a pharmaceutically acceptable salt thereof, wherein:
—R⁹ is selected from —H, —R^{α}—H, —R^{α}—[N(R⁵)₃]Y, —R^{α}—[P(R⁵)₃]Y or —R^{α}—[R⁷]Y;
—R^{α}— is selected from a C₁-C₁₂ alkylene group (preferably a C₁-C₉ alkylene group, preferably a C₂-C₆ alkylene group), wherein one or more (such as one, two, three or four, preferably one or two) carbon atoms in the backbone of the alkylene group may optionally be replaced by a heteroatom or group independently selected from O, S, NH or NMe (preferably O, NH or NMe, preferably O);
—R⁵, each independently, is selected from C₁-C₃ alkyl or phenyl;
—R⁷ is —[NC₅H₅];
Y is a counter anion [preferably Y is a counter anion selected from halide or hexafluorophosphate]; and
q is 0, 1, 2, 3 or 4 (preferably q is 1).

Preferably in the compound or complex according to the first or second aspect of the present invention, the compound or complex is:

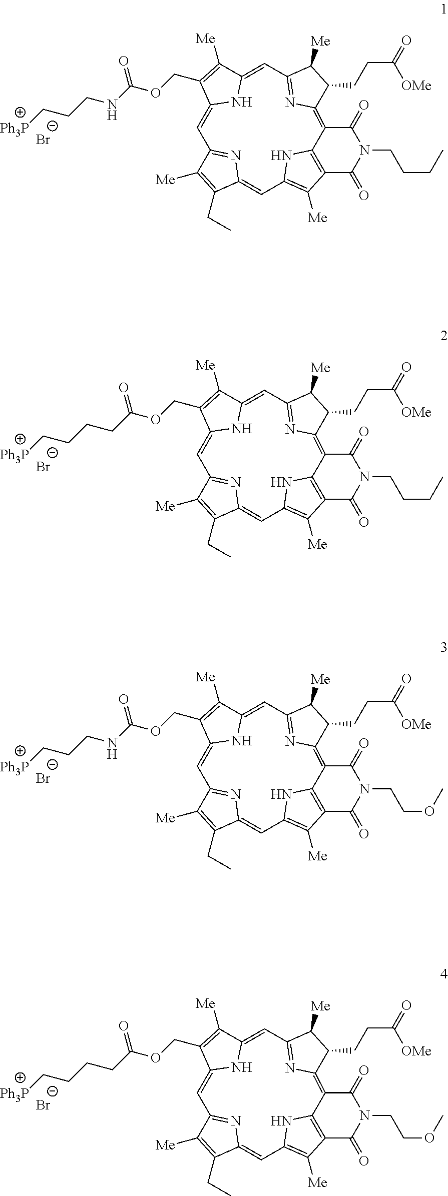

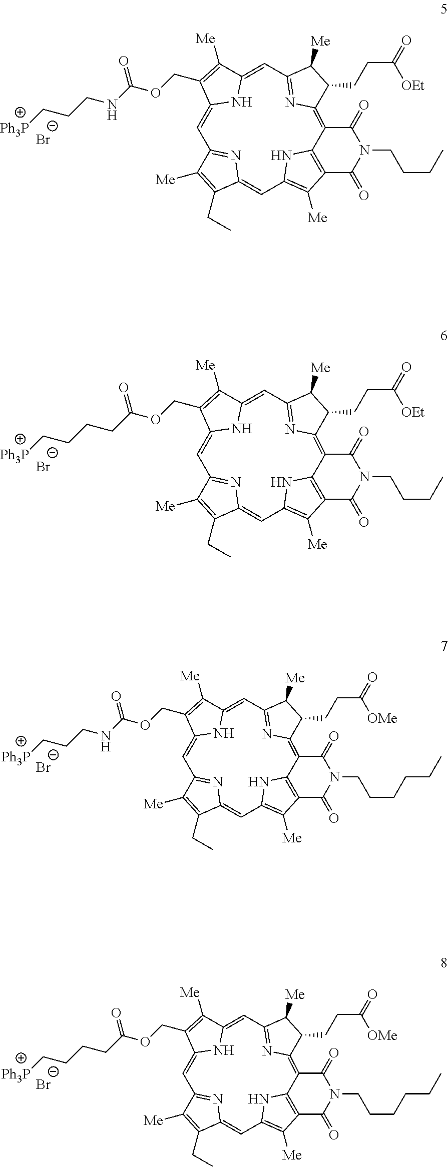

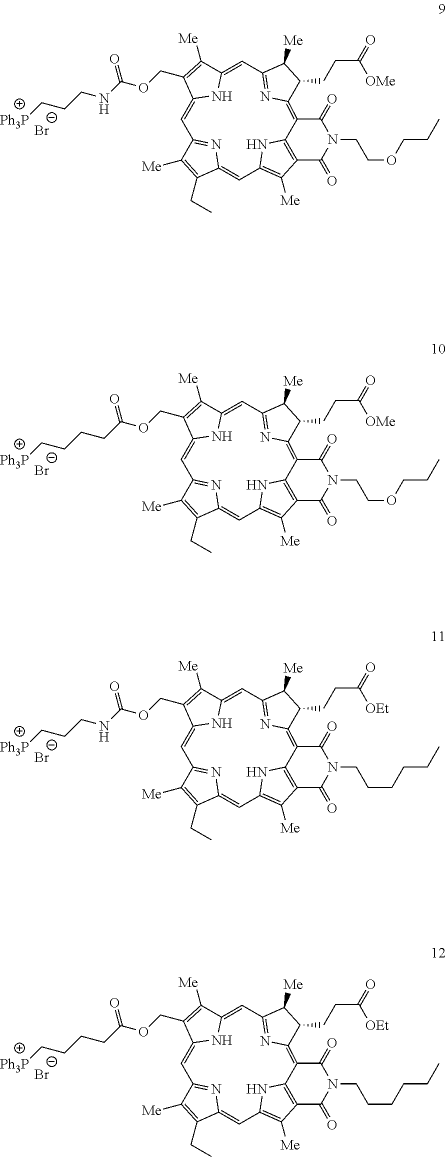

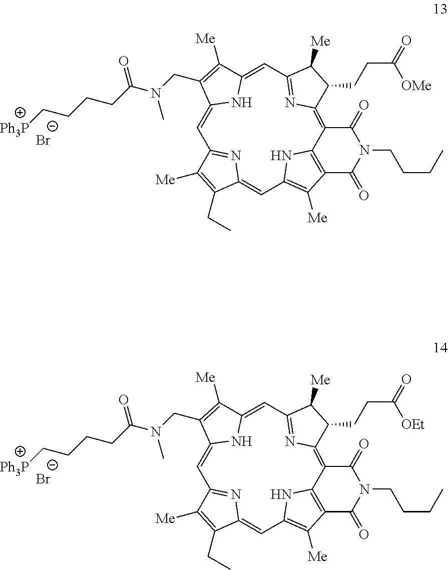

or a metal cation complex thereof, or a pharmaceutically acceptable salt thereof.

Preferably in the compound or complex according to the first or second aspect of the present invention, the compound or complex is:

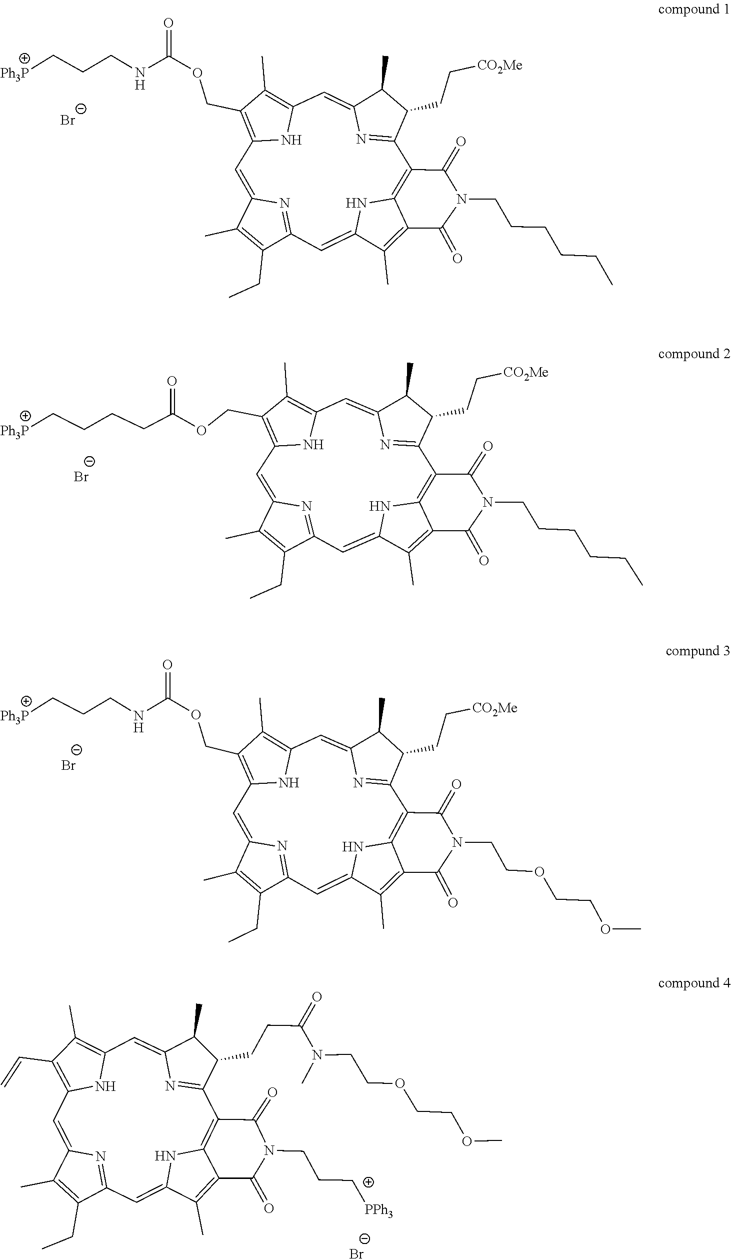

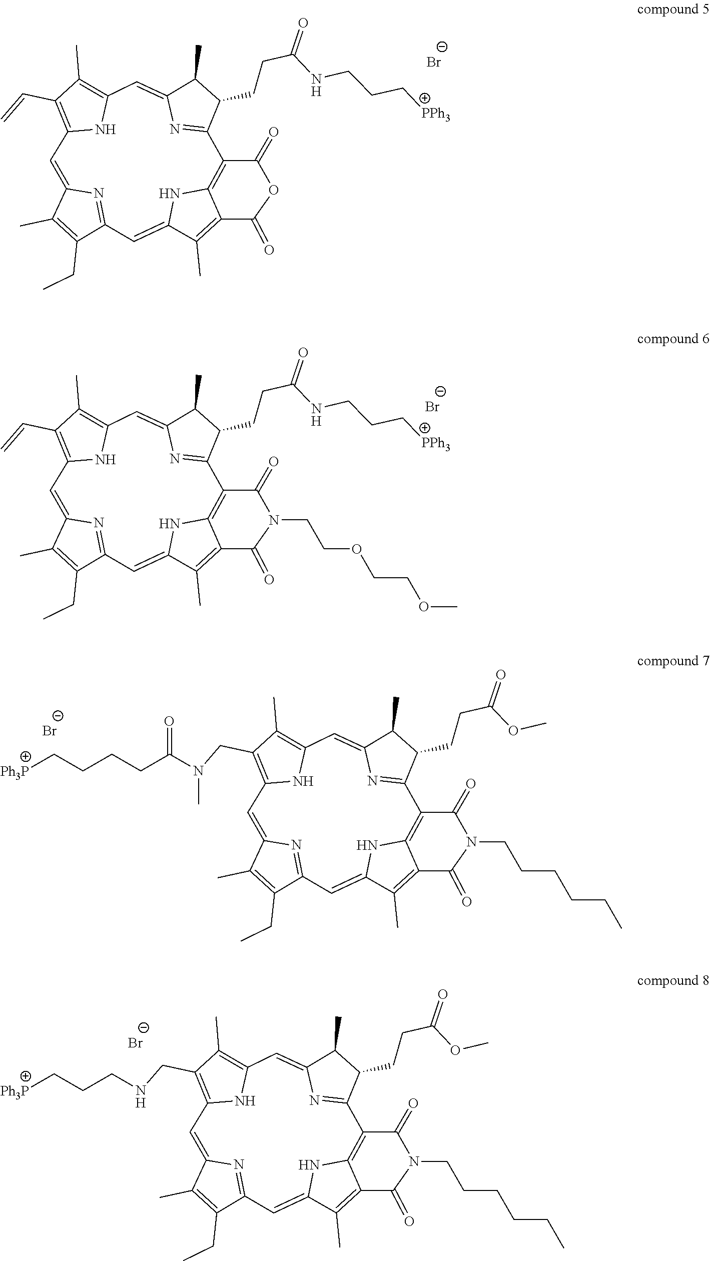

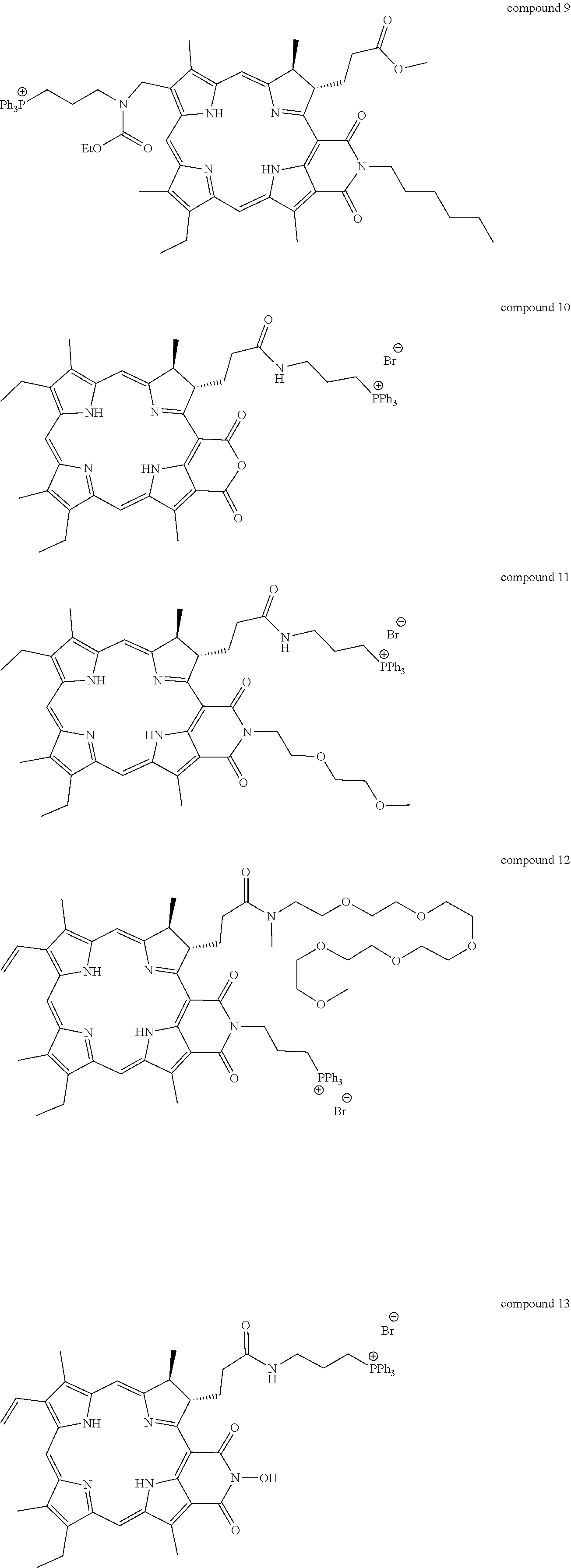

or a metal cation complex thereof, or a pharmaceutically acceptable salt thereof.

In one embodiment, the compound or complex according to the first or second aspect of the invention is in the form of a pharmaceutically acceptable salt. In one embodiment, the compound or complex is in the form of an inorganic salt such as a lithium, sodium, potassium, magnesium, calcium or ammonium salt. In one embodiment, the compound or complex is in the form of a sodium or potassium salt. In one embodiment, the compound is in the form of a sodium salt. In another embodiment, the compound or complex is in the form of an organic salt such as an amine salt (for example a choline or meglumine salt) or an amino acid salt (for example an arginine salt).

The compound or complex according to the first or second aspect of the invention has at least two chiral centres. The compound or complex of the first or second aspect of the invention is preferably substantially enantiomerically pure, which means that the compound or complex comprises less than 10% of other stereoisomers, preferably less than 5%, preferably less than 3%, preferably less than 2%, preferably less than 1%, preferably less than 0.5%, all by weight, as measured by XRPD or SFC.

Preferably, the compound or complex according to the first or second aspect of the invention has a HPLC purity of more than 97%, more preferably more than 98%, more preferably more than 99%, more preferably more than 99.5%, more preferably more than 99.8%, and most preferably more than 99.9%. As used herein the percentage HPLC purity is measured by the area normalisation method.

A third aspect of the invention provides a composition comprising a compound or complex according to the first or second aspect of the invention and a pharmaceutically acceptable carrier or diluent.

In one embodiment, the composition according to the third aspect of the invention further comprises polyvinylpyrrolidone (PVP). In one embodiment, the composition comprises 0.01-10% w/w PVP as a percentage of the total weight of the composition, preferably 0.1-5% w/w PVP as a percentage of the total weight of the composition, preferably 0.5-5% w/w PVP as a percentage of the total weight of the composition. In one embodiment, the PVP is K30.

In one embodiment, the composition according to the third aspect of the invention further comprises dimethylsulfoxide (DMSO). In one embodiment, the composition comprises 0.01-99% w/w DMSO as a percentage of the total weight of the composition, preferably 40-99% w/w DMSO as a percentage of the total weight of the composition, preferably 65-99% w/w DMSO as a percentage of the total weight of the composition.

In one embodiment, the composition according to the third aspect of the invention further comprises an immune checkpoint inhibitor. In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably the compound or complex according to the first or second aspect of the present invention and the pharmaceutical composition according to the third aspect of the present invention are suitable for use in photodynamic therapy or cytoluminescent therapy.

Preferably the compound or complex according to the first or second aspect of the present invention and the pharmaceutical composition according to the third aspect of the present invention are suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the treatment of a disease characterised by benign or malignant cellular hyperproliferation or by areas of neovascularisation.

Preferably the compound or complex according to the first or second aspect of the present invention and the pharmaceutical composition according to the third aspect of the present invention are suitable for the treatment of a benign or malignant tumour.

Preferably the compound or complex according to the first or second aspect of the present invention and the pharmaceutical composition according to the third aspect of the present invention are suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for use in photodynamic diagnosis.

Preferably the compound or complex according to the first or second aspect of the present invention and the pharmaceutical composition according to the third aspect of the present invention are suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the detection of an area that is affected by benign or malignant cellular hyperproliferation or by neovascularisation.

Preferably the compound or complex according to the first or second aspect of the present invention and the pharmaceutical composition according to the third aspect of the present invention are suitable for the detection of a benign or malignant tumour.

Preferably the compound or complex according to the first or second aspect of the present invention and the pharmaceutical composition according to the third aspect of the present invention are suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the fluorescent or phosphorescent detection of the diseases listed above, preferably for the fluorescent or phosphorescent detection and quantification of the said diseases.

Preferably the compound or complex according to the first or second aspect of the present invention and the pharmaceutical composition according to the third aspect of the present invention are adapted for administration simultaneous with or prior to administration of irradiation or sound, preferably for administration prior to administration of irradiation.

If the compound or complex according to the first or second aspect of the present invention or the pharmaceutical composition according to the third aspect of the present invention are for use in photodynamic therapy or cytoluminescent therapy, then they are preferably adapted for administration 5 to 100 hours before the irradiation, preferably 6 to 72 hours before the irradiation, preferably 24 to 48 hours before the irradiation.

If the compound or complex according to the first or second aspect of the present invention or the pharmaceutical composition according to the third aspect of the present invention are for use in photodynamic diagnosis, then they are preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 600 nm to 800 nm, preferably from 640 nm to 750 nm, preferably from 655 nm to 710 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 600 nm to 800 nm, preferably from 640 nm to 750 nm, preferably from 655 nm to 710 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

The pharmaceutical composition according to the third aspect of the present invention may be in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration. The pharmaceutical composition may also be in a form suitable for administration by enema or for administration by injection into a tumour. Preferably the pharmaceutical composition is in a form suitable for oral, parenteral (such as intravenous, intraperitoneal, and intratumoral) or airway administration, preferably in a form suitable for oral or parenteral administration, preferably in a form suitable for oral administration.

In one preferred embodiment, the pharmaceutical composition is in a form suitable for oral administration. Preferably the pharmaceutical composition is provided in the form of a tablet, capsule, hard or soft gelatine capsule, caplet, troche or lozenge, as a powder or granules, or as an aqueous solution, suspension or dispersion. More preferably the pharmaceutical composition is provided in the form of an aqueous solution, suspension or dispersion for oral administration, or alternatively in the form of a freeze-dried powder which can be mixed with water before administration to provide an aqueous solution, suspension or dispersion for oral administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for parenteral administration. Preferably the pharmaceutical composition is in a form suitable for intravenous administration. Preferably the pharmaceutical composition is provided in the form of an aqueous solution for parenteral administration, or alternatively in the form of a freeze-dried powder which can be mixed with water before administration to provide an aqueous solution for parenteral administration. Preferably the pharmaceutical composition is an aqueous solution or suspension having a pH of from 6 to 8.5. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for airway administration. Preferably the pharmaceutical composition is provided in the form of an aqueous solution, suspension or dispersion for airway administration, or alternatively in the form of a freeze-dried powder which can be mixed with water before administration to provide an aqueous solution, suspension or dispersion for airway administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

A fourth aspect of the present invention provides use of a compound or complex according to the first or second aspect of the present invention in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a phototherapeutic agent for use in photodynamic therapy or cytoluminescent therapy. Preferably the phototherapeutic agent is suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the medicament or the phototherapeutic agent of the fourth aspect of the present invention is suitable for the treatment of a disease characterised by benign or malignant cellular hyperproliferation or by areas of neovascularisation.

Preferably the medicament or the phototherapeutic agent of the fourth aspect of the present invention is suitable for the treatment of a benign or malignant tumour.

Preferably the medicament or the phototherapeutic agent of the fourth aspect of the present invention is suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a photodiagnostic agent for use in photodynamic diagnosis.

Preferably the photodiagnostic agent of the fourth aspect of the present invention is suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the detection of an area that is affected by benign or malignant cellular hyperproliferation or by neovascularisation.

Preferably the photodiagnostic agent of the fourth aspect of the present invention is suitable for the detection of a benign or malignant tumour.

Preferably the photodiagnostic agent of the fourth aspect of the present invention is suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the fluorescent or phosphorescent detection of the said diseases, preferably the fluorescent or phosphorescent detection and quantification of the said diseases.

Preferably the medicament, the phototherapeutic agent or the photodiagnostic agent is adapted for administration simultaneous with or prior to administration of irradiation or sound, preferably for administration prior to administration of irradiation.

If the medicament or the phototherapeutic agent is for use in photodynamic therapy or cytoluminescent therapy, then it is preferably adapted for administration 5 to 100 hours before the irradiation, preferably 6 to 72 hours before the irradiation, preferably 24 to 48 hours before the irradiation.

If the photodiagnostic agent is for use in photodynamic diagnosis, then it is preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 600 nm to 800 nm, preferably from 640 nm to 750 nm, preferably from 655 nm to 710 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 600 nm to 800 nm, preferably from 640 nm to 750 nm, preferably from 655 nm to 710 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

A fifth aspect of the present invention provides a method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to the first or second aspect of the present invention to a human or animal in need thereof.

The fifth aspect of the present invention also provides a method of photodynamic therapy or cytoluminescent therapy of a human or animal disease, the method comprising administering a therapeutically effective amount of a compound or complex according to the first or second aspect of the present invention to a human or animal in need thereof. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the method of the fifth aspect of the present invention is a method of treating benign or malignant cellular hyperproliferation or areas of neovascularisation.

Preferably the method of the fifth aspect of the present invention is a method of treating a benign or malignant tumour.

Preferably the method of the fifth aspect of the present invention is a method of treating early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fifth aspect of the present invention also provides a method of photodynamic diagnosis of a human or animal disease, the method comprising administering a diagnostically effective amount of a compound or complex according to the first or second aspect of the present invention to a human or animal. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the human or animal disease is characterised by benign or malignant cellular hyperproliferation or by areas of neovascularisation. Preferably the human or animal disease is a benign or malignant tumour. Preferably the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the method of photodynamic diagnosis is suitable for the fluorescent or phosphorescent detection of the said diseases, preferably for the fluorescent or phosphorescent detection and quantification of the said diseases.

In any of the methods of the fifth aspect of the present invention, the human or animal is preferably further subjected to irradiation or sound simultaneous with or after the administration of the compound or complex according to the first or second aspect of the invention. Preferably the human or animal is subjected to irradiation after the administration of the compound or complex according to the first or second aspect of the invention.

If the method is a method of photodynamic therapy or cytoluminescent therapy, then the human or animal is preferably subjected to irradiation 5 to 100 hours after administration of the compound or complex according to the first or second aspect of the invention, preferably 6 to 72 hours after administration, preferably 24 to 48 hours after administration.

If the method is a method of photodynamic diagnosis, then the human or animal is preferably subjected to irradiation 3 to 60 hours after administration of the compound or complex according to the first or second aspect of the invention, preferably 8 to 40 hours after administration.

Preferably the irradiation is electromagnetic radiation with a wavelength in the range of from 500 nm to 1000 nm, preferably from 600 nm to 800 nm, preferably from 640 nm to 750 nm, preferably from 655 nm to 710 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 600 nm to 800 nm, preferably from 640 nm to 750 nm, preferably from 655 nm to 710 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

In any of the methods of the fifth aspect of the present invention, preferably the human or animal is a human.

A sixth aspect of the present invention provides a pharmaceutical combination or kit comprising:
(a) a compound or complex according to the first or second aspect of the present invention; and
(b) an immune checkpoint inhibitor.

In one embodiment, the immune checkpoint inhibitor is an inhibitor of PD-1 (programmed cell death protein 1), PD-Li (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably, the combination or kit of the sixth aspect is for use in the treatment of a disease, disorder or condition, wherein the disease, disorder or condition is responsive to PD-1, PD-Li or CTLA4 inhibition. Preferably, the combination or kit of the sixth aspect is for use in the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect also provides a use of the combination or kit of the sixth aspect of the invention in the manufacture of a medicament for the treatment of a disease, disorder or condition which is responsive to PD-1, PD-Li or CTLA4 inhibition. The sixth aspect also provides a use of the combination or kit of the sixth aspect of the invention in the manufacture of a medicament for the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect of the invention also provides a method of treating a disease, disorder or condition which is responsive to PD-1, PD-Li or CTLA4 inhibition, the method comprising administering a therapeutically effective amount of the combination or kit of the sixth aspect of the present invention to a human or animal in need thereof. The sixth aspect of the invention also provides a method of treating cancer, the method comprising administering a therapeutically effective amount of the combination or kit of the sixth aspect of the present invention to a human or animal in need thereof. In one embodiment, the cancer is melanoma, lung cancer (e.g. non small cell lung cancer), kidney cancer, bladder cancer, head and neck cancer, or Hodgkin's lymphoma.

For the combination or kit of the sixth aspect of the invention, the compound or complex according to the first or second aspect of the invention, and the immune checkpoint inhibitor may be provided together in one pharmaceutical composition or separately in two pharmaceutical compositions. If provided in two pharmaceutical compositions, these may be administered at the same time or at different times.

Preferably the combination or kit of the sixth aspect is adapted for administration simultaneous with or prior to administration of irradiation or sound, preferably for administration prior to administration of irradiation. In one embodiment, the combination or kit of the sixth aspect is adapted for administration 5 to 100 hours before the irradiation, preferably 6 to 72 hours before the irradiation, preferably 24 to 48 hours before the irradiation.

Preferably the irradiation used in the photodynamic therapy or cytoluminescent therapy is electromagnetic radiation with a wavelength in the range of from 500 nm to 1000 nm, preferably from 600 nm to 800 nm, preferably from 640 nm to 750 nm, preferably from 655 nm to 710 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 600 nm to 800 nm, preferably from 640 nm to 750 nm, preferably from 655 nm to 710 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### Prophetic Synthetic Experimental Details

Compounds according to the first or second aspect of the present invention can be prepared according to the general synthesis below. Examples of suitable reagents and conditions for the functional group conversions can be found in WO2022/112537, PCT/EP2022/083551, PCT/EP2022/083553 and PCT/EP2022/083554, all of which are incorporated herein by reference in their entirety.

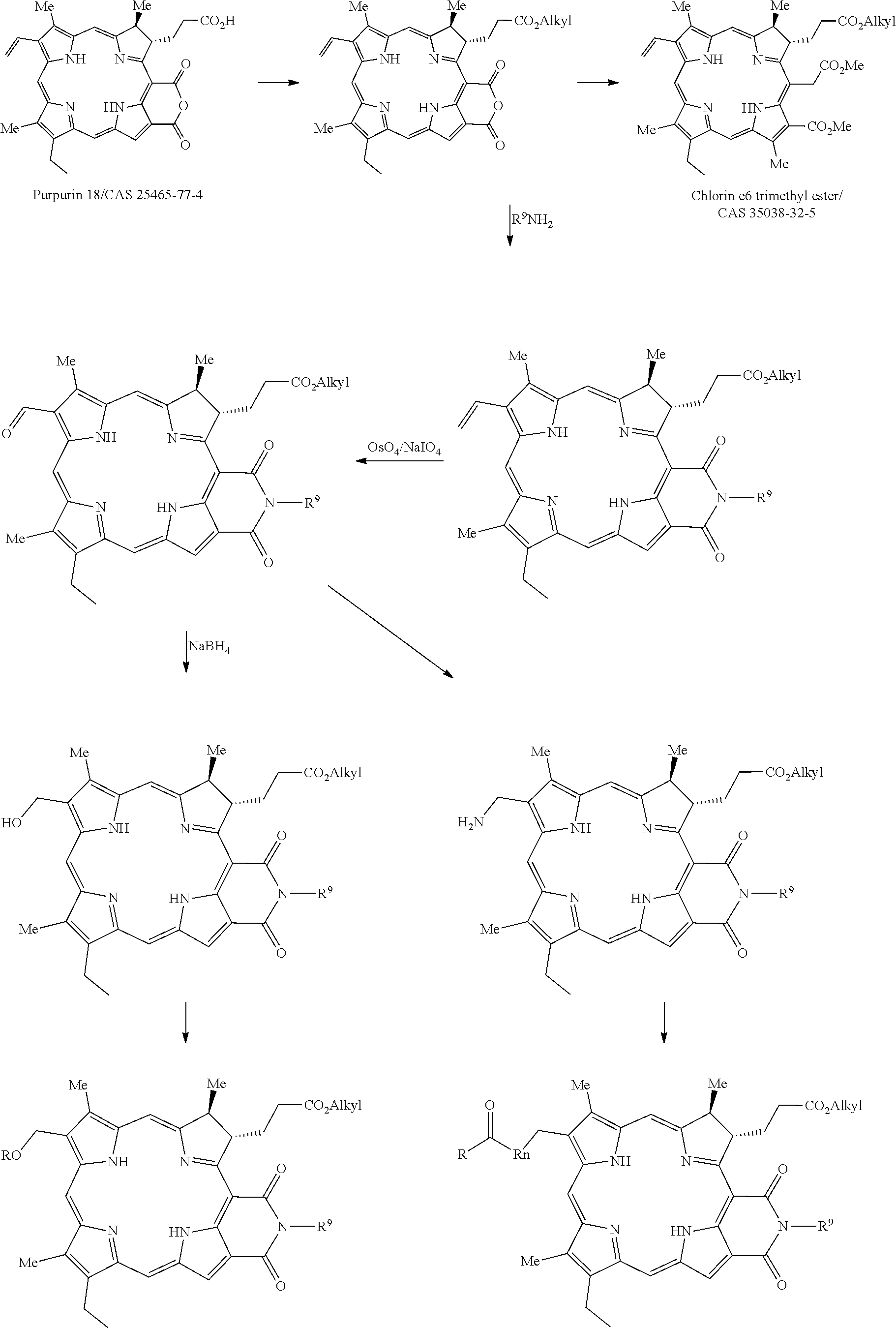

### Synthetic Experimental Details

### Synthesis Example 1—Synthesis of purpurin 18 13-(N-(3-triphenylphosphoniumpropyl)bromide)carbamate N-hexylimide methyl ester (compound 1)

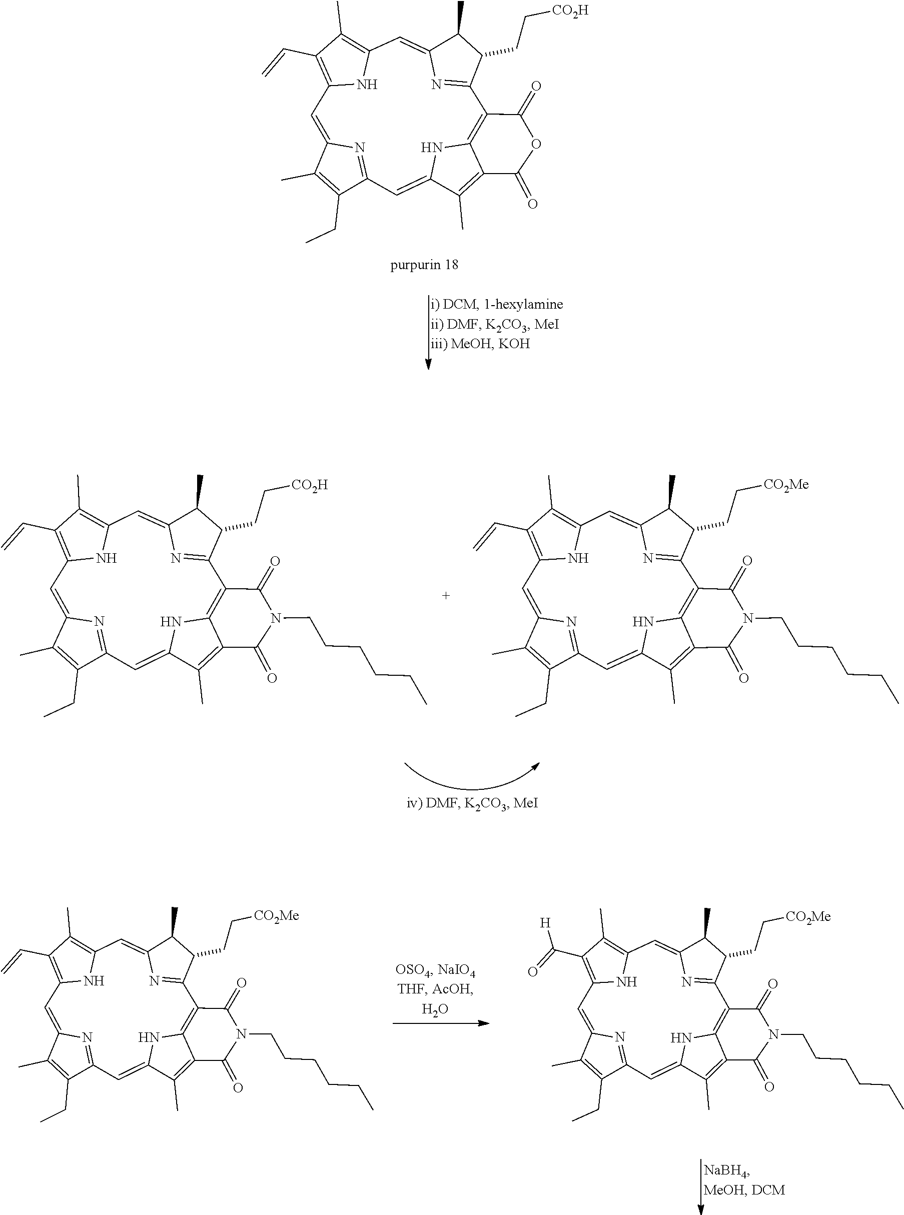

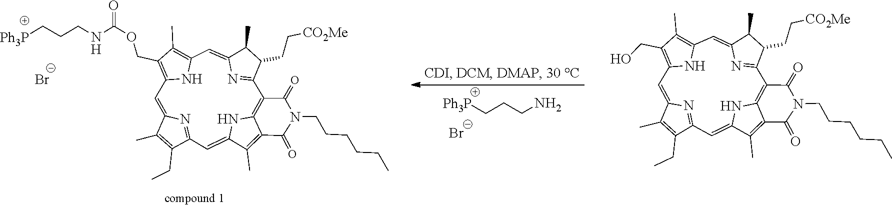

Step 1: To a 25 mL RBF was added purpurin 18 (100 mg, 0.177 mmol, 1 eq), 1-hexylamine (135 mg, 1.334 mmol, 7.5 eq) and DCM (4 mL). The resultant mixture was stirred under nitrogen at 20 °C for 16 hours. The reaction mixture was concentrated by rotary evaporation and further dried under high vacuum (0.1 mBar) to give the crude amide isomers as a green residue that was used directly in the next step.

Step 2: To a 25 mL RBF was added purpurin 18 hexyl amide diacid (118 mg, 0.177 mmol, 1 eq), DMF (3 mL), potassium carbonate (98 mg, 0.708 mmol, 4 eq) and methyl iodide (101 mg, 0.708 mmol, 4 eq). The resultant mixture was stirred under nitrogen at °C. After 30 minutes DCM (0.5 mL) was added and stirring was continued. After 3 hours the reaction mixture was diluted with EtOAc (30 mL), washed with water (3×10 mL), dried (Na₂SO₄) and concentrated by rotary evaporation to give the crude dimethyl ester isomers as a green residue (~150 mg) that was used directly in the next step.

Step 3: To a 25 mL RBF was added purpurin 18 hexyl amide dimethyl ester (123 mg, 0.177 mmol, 1 eq), MeOH (2 mL) and 10% KOH/MeOH (1 mL). The resultant mixture was stirred under nitrogen at 20 °C for 3 minutes. The reaction mixture was diluted with DCM (20 mL), washed with pH 7 phosphate buffer (20 mL) then water (20 mL), dried (Na₂SO₄) and concentrated by rotary evaporation to give the crude product (106 mg). The residue was purified by column chromatography using a gradient of 1-3% MeOH/DCM. Fractions containing a red product (R_{f}=0.84 in 5% MeOH/DCM) were combined to give purpurin 18 N-hexylimide methyl ester as a dark red solid (15 mg). Fractions containing a red product (R_{f}=0.40 in 5% MeOH/DCM) were combined to give purpurin 18 N-hexylimide as a dark red solid (60 mg).

Step 4: Purpurin 18 N-hexylimide (60 mg) was dissolved in DMF (1 mL) and potassium carbonate (90 mg) and methyl iodide (100 mg) added. The resultant mixture was stirred under nitrogen at 30 °C for 1 hour. The reaction mixture was diluted with EtOAc (20 mL), washed with water (3×10 mL), dried (Na₂SO₄) and concentrated by rotary evaporation to give purpurin 18 N-hexylimide methyl ester as a red solid (combined yield 75 mg, 64% over 4 steps).

¹H NMR (400 MHz, CDCl₃) δ 9.42 (s, 1H), 9.20 (s, 1H), 8.58 (s, 1H), 7.80 (dd, 1H), 6.22 (d, 1H), 6.10 (d, 1H), 5.41 (m, 1H), 4.51-4.45 (m, 2H), 4.37 (q, 1H), 3.74 (s, 3H), 3.58 (s, 3H), 3.52-3.45 (m, 2H), 3.32 (s, 3H), 3.03 (s, 3H), 2.78-2.68 (m, 1H), 2.50-2.33 (m, 2H), 2.08-1.97 (m, 3H), 1.79 (d, 3H), 1.68-1.56 (m, 6H), 1.54-1.40 (m, 4H), 0.97 (t, 3H), −0.23 (brs, 1H), −0.32 (brs, 1H).

Step 5: To a 25 mL RBF was added purpurin 18 N-hexylimide methyl ester (75 mg, 0.113 mmol, 1 eq), THF (3 mL), osmium tetroxide (0.3 mg, 0.001 mmol, 0.01 eq), deionized water (0.2 mL), AcOH (0.2 mL) and sodium periodate (63 mg, 0.295 mmol, 2.6 eq). The resultant mixture was stirred under nitrogen in the dark at ambient temperature for 17 hours. The reaction mixture was concentrated using a rotary evaporator to remove the THF and then re-dissolved in DCM (15 mL), transferred to a separatory funnel and washed with brine (10 mL), saturated NaHCO₃ (10 mL) and water (10 mL) before being dried (Na₂SO₄) and concentrated by rotary evaporation to give purpurin 18 13-formyl N-hexylimide methyl ester as a powdery brown solid (95 mg, quantitative) that was not purified further.

¹H NMR (400 MHz, CDCl₃) δ 11.39 (s, 1H), 10.11 (s, 1H), 9.61 (s, 1H), 8.73 (s, 1H), 5.41 (d, 1H), 4.49-4.37 (m, 3H), 3.83 (s, 3H), 3.67 (s, 3H), 3.63 (q, 2H), 3.57 (s, 3H), 3.20 (s, 3H), 2.75-2.68 (m, 1H), 2.48-2.35 (m, 2H), 2.04-1.94 (m, 3H), 1.78 (d, 3H), 1.68-1.53 (m, 8H), 1.52-1.39 (m, 6H), 0.94 (t, 3H), −0.29 (brs, 1H), −0.48 (brs, 1H).

Step 6: To a 25 mL RBF was added purpurin 18 13-formyl N-hexylimide methyl ester (75 mg, 0.113 mmol, 1 eq), MeOH (2 mL), DCM (1 mL) and sodium borohydride (9 mg, 0.226 mmol, 2 eq). The resultant mixture was stirred under nitrogen at ambient temperature for 10 minutes. The reaction mixture was diluted with water (5 mL) and stirred for 10 minutes. The mixture was then diluted with pH=7 phosphate buffer (10 mL) and extracted with DCM (2×15 mL). The combined DCM layers were washed with water (50 mL) before being dried (Na₂SO₄) and concentrated by rotary evaporation to give a dark red solid (~135 mg). The residue was purified by column chromatography using a gradient of 1-2% MeOH/DCM. Fractions containing the major red spot (R_{f}=0.55 in 5% MeOH/DCM) were combined to give purpurin 18 13-hydroxymethyl N-hexylimide methyl ester as a red solid (51 mg, 68%).

¹H NMR (400 MHz, CDCl₃) δ 9.54 (s, 1H), 9.41 (s, 1H), 8.58 (s, 1H), 5.80 (s, 2H), 5.39 (dd, 1H), 4.48-4.41 (m, 2H), 4.34 (q, 1H), 3.78 (s, 3H), 3.61 (q, 2H), 3.56 (s, 3H), 3.36 (s, 3H), 3.17 (s, 3H), 2.72-2.64 (m, 1H), 2.46-2.29 (m, 3H), 2.02-1.93 (m, 4H), 1.76 (d, 3H), 1.68-1.53 (m, 10H), 1.51-1.37 (m, 6H), 0.94 (t, 3H), −0.36 (brs, 2H).

Step 7: To a 25 mL RBF was added purpurin 18 13-hydroxymethyl N-hexylimide methyl ester (45 mg, 0.0676 mmol, 1 eq), carbonyl diimidazole (CDI) (22 mg, 0.1352 mmol, 2 eq), DCM (2 mL) and DMAP (3 mg). The resultant mixture was stirred under nitrogen for 2 hours at 30 °C. (3-Aminopropyl)triphenylphosphonium bromide (135 mg, 0.3379 mmol, 5 eq) was added and stirring was continued for 16 hours at 30 °C. The reaction mixture was diluted with DCM (15 mL), transferred to a separatory funnel and washed with water (15 mL). The organic layer was separated, before being dried (Na₂SO₄) and concentrated by rotary evaporation to give a dark red residue. The residue was purified by column chromatography using a gradient of 3-6% MeOH/DCM. Fractions containing the major dark red spot (R_{f}=0.15 in 7% MeOH/DCM) were combined to give compound 1 as a dark red solid (53 mg, 72%).

¹H NMR (400 MHz, CDCl₃) δ 9.57 (s, 1H), 9.55 (s, 1H), 8.58 (s, 1H), 7.71 (m, 1H), 7.59-7.51 (m, 6H), 7.41-7.30 (m, 9H), 6.22 (m, 2H), 5.42-5.38 (m, 1H), 4.51-4.40 (m, 2H), 4.38-4.31 (m, 1H), 3.81 (s, 3H), 3.78-3.68 (m, 2H), 3.52-3.50 (m, 7H), 3.40 (s, 3H), 3.19 (s, 3H), 2.74-2.65 (m, 1H), 2.47-2.25 (m, 2H), 2.03-1.92 (m, 3H), 1.81-1.70 (m, 8H), 1.66-1.57 (m, 5H), 1.50-1.38 (m, 5H), 1.30-1.19 (m, 3H), 0.94 (t, 3H), −0.23 (brs, 1H), −0.30 (brs, 1H).

### Synthesis Example 2—Synthesis of purpurin 18 N-hexylimide 13-(oxopentyl)triphenylphosphonium bromide (compound 2)

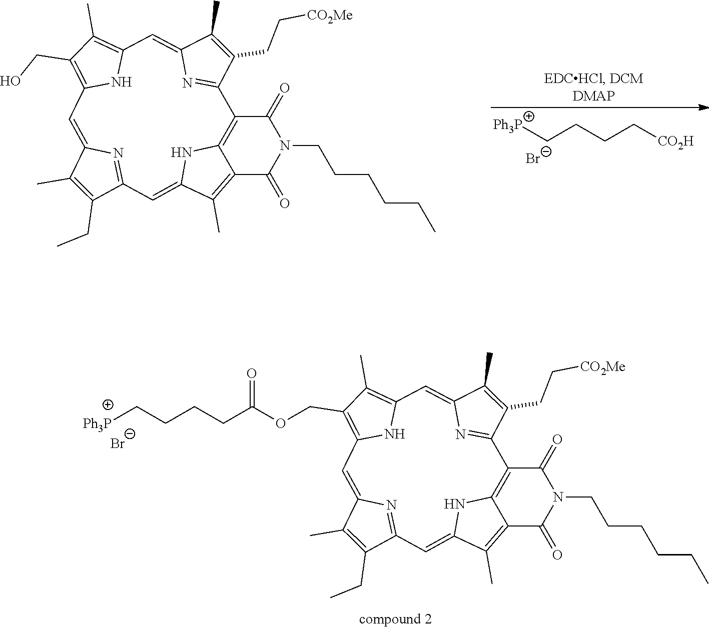

To a 25 mL RBF was added purpurin 18 13-hydroxymethyl N-hexylimide methyl ester (33 mg, 0.0496 mmol, 1 eq), 4-(carboxybutyl)triphenylphosphonium bromide (44 mg, 0.0991 mmol, 2 eq), EDC.HCl (19 mg, 0.0991 mmol, 2 eq), DMAP (12 mg, 0.0991 mmol, 2 eq) and DCM (3 mL). The resultant mixture was stirred under nitrogen at ambient temperature. After 2 hours the solvent was removed by rotary evaporation to leave a red oil. The residue was purified by column chromatography using a gradient of 3-5% MeOH/DCM. Fractions containing the major dark red spot (R_{f}=0.15 in 5% MeOH/DCM) were combined to give compound 2 as a dark red solid (47 mg, 87%).

¹H NMR (400 MHz, CDCl₃) δ 9.61 (s, 1H), 9.39 (s, 1H), 8.61 (s, 1H), 7.63-7.57 (m, 6H), 7.50-7.45 (m, 3H), 7.41-7.36 (m, 6H), 6.21 (m, 2H), 5.43-5.38 (m, 1H), 4.50-4.41 (m, 2H), 4.40-4.34 (m, 1H), 3.87-3.77 (m, 5H), 3.56 (s, 3H), 3.35 (s, 3H), 3.17 (s, 3H), 2.75-2.67 (m, 1H), 2.59-2.54 (m, 2H), 2.48-2.32 (m, 2H), 2.13-2.05 (m, 2H), 2.02-1.92 (m, 3H), 1.79-1.70 (m, 7H), 1.68-1.52 (m, 8H), 1.51-1.38 (m, 5H), 1.30-1.18 (m, 3H), 0.94 (t, 3H), −0.32 (brs, 1H), −0.40 (brs, 1H).

### Synthesis Example 3—Synthesis of purpurin 18 13-(N-(3-triphenylphosphoniumpropyl)bromide)carbamate N-(2-(2-methoxyethoxy)ethyl)imide methyl ester (compound 3)

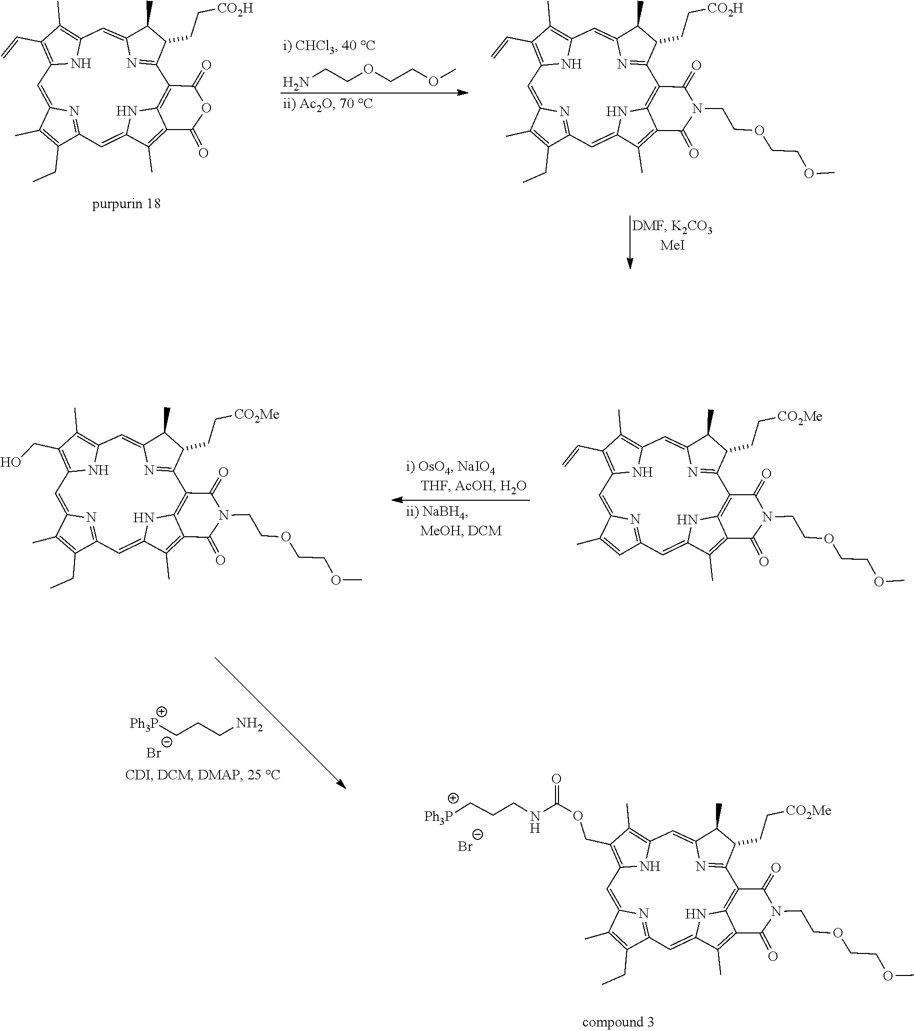

Step 1: To a 25 mL RBF was added purpurin 18 (100 mg, 0.177 mmol, 1 eq), 2-(methoxyethoxy)ethanamine (158 mg, 1.328 mmol, 7.5 eq) and DCM (4 mL). The resultant mixture was stirred under nitrogen at 30 °C for 16 hours. The reaction mixture was concentrated by rotary evaporation and to the resulting residue was added acetic anhydride (1.5 mL) and the mixture was stirred with heating at 70 °C for 2 hours. The solution was cooled and 5% NaHCO₃ (4 mL) was added and the mixture stirred for 2 hours. Water (10 mL) was added and the mixture was extracted with DCM (3×5 mL). The combined organics were dried (Na₂SO₄) and concentrated by rotary evaporation to give the crude imide as a red residue (~180 mg). The residue was purified by column chromatography using a gradient of 1.5-3% MeOH/DCM. Fractions containing the major red spot (R_{f}=0.10 in 5% MeOH/DCM) were combined to give purpurin 18 N-(2-(2-methoxyethoxy)ethyl)imide as a dark red solid (66 mg, 56%).

¹H NMR (400 MHz, CDCl₃) δ 9.51-9.39 (m, 1H), 9.28-9.22 (m, 1H), 8.53-8.51 (m, 1H), 7.87-7.76 (m, 1H), 6.23 (d, 1H), 6.12 (m, 1H), 5.30 (m, 1H), 4.76-4.65 (m, 1H), 4.38-4.28 (m, 1H), 4.08-3.99 (m, 1H), 3.85-3.78 (m, 1H), 3.72 (s, 2H), 3.68-3.50 (m, 9H), 3.37 (s, 2H), 3.31-3.27 (m, 5H), 3.11-3.07 (m, 3H), 2.80-2.69 (m, 1H), 2.50-2.35 (m, 2H), 2.00-1.88 (m, 1H), 1.71 (d, 3H), 1.65-1.57 (m, 3H), −0.13 (brs, 1H).

Step 2: To a 25 mL RBF was added purpurin 18 N-(2-(2-methoxyethoxy)ethyl)imide (66 mg, 0.099 mmol, 1 eq), DMF (1 mL), potassium carbonate (120 mg, 0.868 mmol, 8.8 eq) and methyl iodide (100 mg, 0.707 mmol, 7.1 eq). The resultant mixture was stirred under nitrogen at 30 °C. After 3 hours the reaction mixture was diluted with EtOAc (15 mL), washed with water (3×10 mL) before being dried (Na₂SO₄) and concentrated by rotary evaporation to give the crude methyl ester as a red residue (~60 mg). The residue was purified by column chromatography using a gradient of 1-2% MeOH/DCM. Fractions containing the major red spot (R_{f}=0.45 in 5% MeOH/DCM) were combined to give purpurin 18 N-(2-(2-methoxyethoxy)ethyl)imide methyl ester as a dark red solid (42 mg, 63%).

¹H NMR (400 MHz, CDCl₃) δ 9.54 (s, 1H), 9.31 (s, 1H), 8.58 (s, 1H), 7.88 (dd, 1H), 6.28 (d, 1H), 6.14 (d, 1H), 5.33 (m, 1H), 4.77-4.72 (m, 2H), 4.36 (q, 1H), 4.10-4.01 (m, 2H), 3.88-3.85 (m, 2H), 3.78 (s, 3H), 3.65-3.59 (m, 5H), 3.58 (s, 3H), 3.36 (s, 3H), 3.34 (s, 3H), 3.13 (s, 3H), 2.75-2.67 (m, 1H), 2.50-2.35 (m, 2H), 2.02-1.94 (m, 1H), 1.75 (d, 3H), 1.68-1.58 (m, 6H), −0.10 (brs, 1H), −0.19 (brs, 1H).

Step 3: To a 25 mL RBF was added purpurin 18 N-(2-(2-methoxyethoxy)ethyl)imide methyl ester (42 mg, 0.0618 mmol, 1 eq), THF (3 mL), osmium tetroxide (0.3 mg, 0.001 mmol, 0.01 eq), deionized water (0.2 mL), AcOH (0.2 mL) and sodium periodate (63 mg, 0.295 mmol, 2.6 eq). The resultant mixture was stirred under nitrogen in the dark at ambient temperature for 17 hours. The reaction mixture was concentrated using a rotary evaporator to remove the THF and then re-dissolved in DCM (15 mL), transferred to a separatory funnel and washed with brine (10 mL), saturated NaHCO₃ (10 mL) and water (10 mL) before being dried (Na₂SO₄) and concentrated by rotary evaporation to give a powdery brown solid (95 mg). The crude aldehyde was dissolved in MeOH (2 mL) and DCM (1 mL) and sodium borohydride (6 mg, 0.159 mmol, 2.6 eq) was added. The resultant mixture was stirred under nitrogen at ambient temperature for 5 minutes. The reaction mixture was diluted with water (5 mL) and stirred for 10 minutes. The mixture was then diluted with pH=7 phosphate buffer (10 mL) and extracted with DCM (2×15 mL). The combined DCM layers washed with water (50 mL), dried (Na₂SO₄) and concentrated by rotary evaporation to give a dark red solid (~45 mg). The residue was purified by column chromatography using a gradient of 1-2.5% MeOH/DCM. Fractions containing the major red spot (R_{f}=0.45 in 5% MeOH/DCM) were combined to give purpurin 18 13-hydroxymethyl N-(2-(2-methoxyethoxy)ethyl)imide methyl ester as a dark red solid (16 mg, 38%).

¹H NMR (400 MHz, CDCl₃) δ 9.47 (s, 1H), 9.38 (s, 1H), 8.56 (s, 1H), 5.78 (s, 2H), 5.31 (dd, 1H), 4.77-4.71 (m, 2H), 4.33 (q, 1H), 4.11-4.01 (m, 2H), 3.88-3.85 (m, 2H), 3.72 (s, 3H), 3.65-3.60 (m, 5H), 3.60-3.54 (m, 5H), 3.36 (s, 3H), 3.34 (s, 3H), 3.14 (s, 3H), 2.75-2.66 (m, 1H), 2.45-2.32 (m, 2H), 2.23 (brs, 1H), 1.98-1.89 (m, 1H), 1.74 (d, 3H), 1.65-1.48 (m, 6H), −0.38 (brs, 2H).

Step 4: To a 25 mL RBF was added purpurin 18 13-hydroxymethyl N-(2-(2-methoxyethoxy)ethyl)imide methyl ester (16 mg, 0.0234 mmol, 1 eq), carbonyl diimidazole (8 mg, 0.0468 mmol, 2 eq), DCM (1 mL) and DMAP (1 mg). The resultant mixture was stirred under nitrogen for 2 hours at 25 °C. (3-Aminopropyl)triphenylphosphonium bromide (47 mg, 0.1170 mmol, 5 eq) was added and stirring was continued for 16 hours at 25 °C. The reaction mixture was diluted with DCM (10 mL), transferred to a separatory funnel and washed with water (1 mL), dried (Na₂SO₄) and concentrated by rotary evaporation to give a dark red residue. The residue was purified by column chromatography using a gradient of 3-6% MeOH/DCM. Fractions containing the major dark red spot (R_{f}=0.25 in 7% MeOH/DCM) were combined to give compound 3 as a dark red solid (19 mg, 73%).

¹H NMR (400 MHz, CDCl₃) δ 9.57 (m, 2H), 8.57 (s, 1H), 7.77 (m, 1H), 7.58-7.50 (m, 6H), 7.40-7.29 (m, 9H), 6.22 (m, 2H), 5.36-5.31 (m, 1H), 4.78-4.71 (m, 2H), 4.37-4.30 (m, 1H), 4.10-4.04 (m, 2H), 3.88-3.85 (m, 2H), 3.80 (s, 3H), 3.76-3.67 (m, 2H), 3.63-3.55 (m, 7H), 3.55-3.49 (m, 2H), 3.40 (s, 3H), 3.36 (s, 3H), 3.19 (s, 3H), 2.76-2.68 (m, 1H), 2.47-2.35 (m, 2H), 2.00-1.88 (m, 1H), 1.87-1.70 (m, 9H), 1.66-1.60 (m, 3H), −0.17 (brs, 1H), −0.26 (brs, 1H).

### Synthesis Example 4—Synthesis of purpurin 18 N-(3-triphenylphosphoniumpropylbromide)imide N-(2-(2-methoxyethoxy)ethyl)-N-methyl-amide (compound 4)

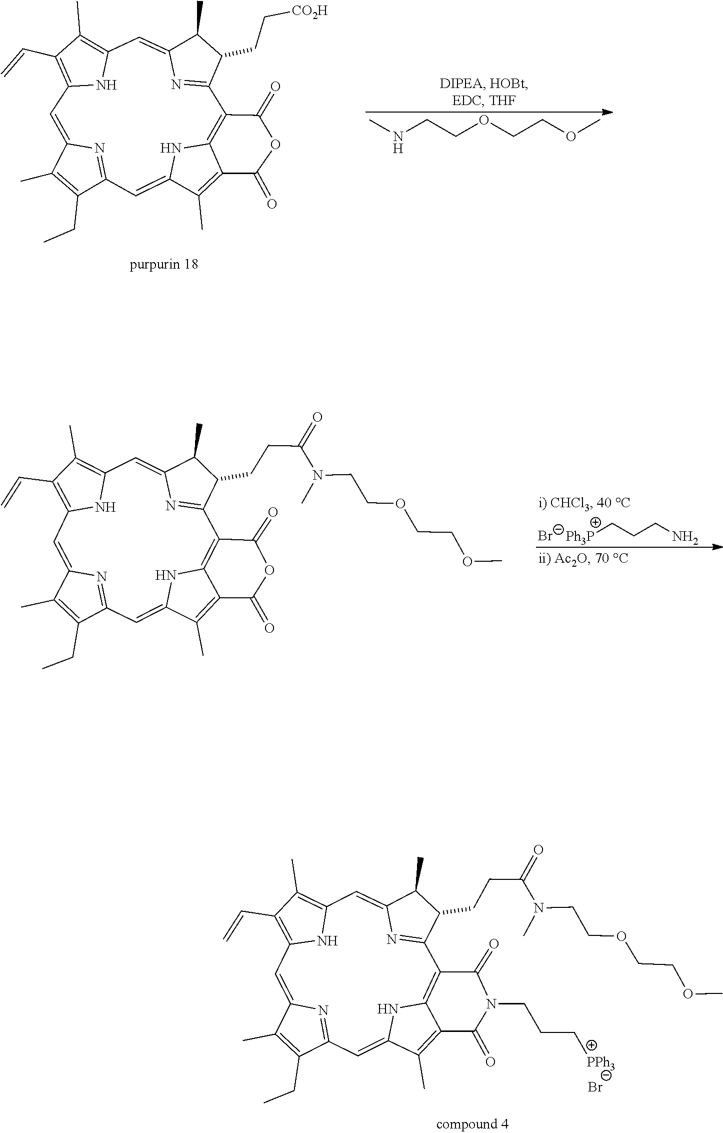

Step 1: A 1N 25 mL RBF was charged with purpurin 18 (300 mg, 0.531 mmol, 1 eq), 2-(2-methoxyethoxy)-N-methylethan-1-amine (113 mg, 0.850 mmol, 1.6 eq), DIPEA (137 mg, 1.06 mmol, 2 eq), HOBt (81 mg, 0.531 mmol, 1 eq) and THF (12 mL). The mixture was stirred under nitrogen for 5 minutes, after which EDC (153 mg, 0.797 mmol, 1.5 eq) was added. The resultant solution was stirred for 18 hours under a nitrogen atmosphere at 25 °C. The reaction progress was monitored by HPLC. The reaction mixture was then concentrated by rotary evaporation and re-dissolved in DCM (50 mL). The resultant solution was transferred to a separating funnel and washed with 0.5M HCl (2×50 mL), then pH7 buffer solution (30 mL). The organic layer was then dried over Na₂SO₄, filtered on a sintered glass funnel and concentrated by rotary evaporation to give the crude product as a dark red solid. The crude product was purified by column chromatography (silica gel, 4×24 cm). The product was loaded on the top of the column as a solution of the crude product in DCM. An eluent of 2% MeOH in DCM was used to elute the product band from the column. Fractions containing a red-brown spot by TLC analysis at R_{f}=0.3 in 5% MeOH/DCM were combined and concentrated by rotary evaporation to give purpurin 18 N-(2-(2-methoxyethoxy)ethyl)-N-methyl-amide as a dark red-brown solid (185 mg, 51%). (94.51% purity by HPLC).

¹H NMR (400 MHz, Chloroform-d) δ 9.44 (d, J=2.5 Hz, 1H), 9.27 (d, J=3.1 Hz, 1H), 8.57 (s, 1H), 7.84 (dd, J=17.8, 11.6 Hz, 1H), 6.27 (dt, J=17.8, 1.1 Hz, 1H), 6.17 (dt, J=11.5, 1.2 Hz, 1H), 5.12 (td, J=9.6, 2.4 Hz, 1H), 4.54-4.30 (m, 1H), 3.69 (s, 3H), 3.61-3.50 (m, 5H), 3.50-3.44 (m, 2H), 3.35-3.29 (m, 6H), 3.10 (s, 2H), 3.09 (d, J=1.3 Hz, 3H), 2.92 (s, 2H), 2.75-2.57 (m, 1H), 2.41 (ddt, J=13.2, 11.3, 2.8 Hz, 1H), 2.07-1.89 (m, 1H), 1.74 (dd, J=7.3, 2.5 Hz, 3H), 1.61 (t, J=7.6 Hz, 3H), 0.20 (s, 1H), −0.09 (s, 1H).

Step 2: To a 25 mL RBF was added purpurin 18 N-(2-(2-methoxyethoxy)ethyl)-N-methyl-amide (145 mg, 0.213 mmol, 1 eq), (3-aminopropyl)triphenylphosphonium bromide (342 mg, 0.853 mmol, 4 eq) and chloroform (5 mL). The resultant mixture was stirred under nitrogen at 40 °C for 18 hours. The reaction mixture was concentrated by rotary evaporation and to the resulting residue was added acetic anhydride (1.5 mL) and the mixture was stirred with heating at 70 °C for 2 hours. The solution was cooled and 5% NaHCO₃ (5 mL) was added and the mixture stirred for 2 hours. Water (10 mL) was added and the mixture was extracted with DCM (3×10 mL). The combined organics were dried (Na₂SO₄) and concentrated by rotary evaporation to give the crude imide as a red residue (~400 mg). The residue was purified by column chromatography eluting using a gradient of 5-8% MeOH/DCM. Fractions with a red spot at R_{f}=0.3 in 8% MeOH/DCM were collected and concentrated to give compound 4 as a dark red solid (102 mg, 45% yield).

¹H NMR (400 MHz, CDCl₃) δ 9.45 (s, 1H), 9.30 (s, 1H), 8.56 (s, 1H), 8.02-7.96 (m, 1H), 7.90-7.80 (m, 7H), 7.75-7.60 (m, 10H), 7.55-7.50 (m, 1H), 6.29 (d, 1H), 6.17 (d, 1H), 5.21 (m, 1H), 4.69-4.64 (m, 1H), 4.41-4.27 (m, 4H), 4.09-4.00 (m, 2H), 3.68 (s, 3H), 3.58 (q, 2H), 3.55-3.40 (m, 2H), 3.36 (m, 6H), 3.27-3.23 (m, 4H), 3.21-3.16 (m, 3H), 3.15-3.10 (m, 5H), 2.62 (s, 2H), 2.50-2.35 (m, 5H), 2.35-2.25 (m, 1H), 2.00-1.80 (m, 5H), 1.75 (m, 5H), 1.62 (m, 4H), 0.05-−0.10 (br m, 2H).

### Synthesis Example 5—Synthesis of purpurin 18 (N-(3-triphenylphosphoniumpropyl)bromide)amide (compound 5)

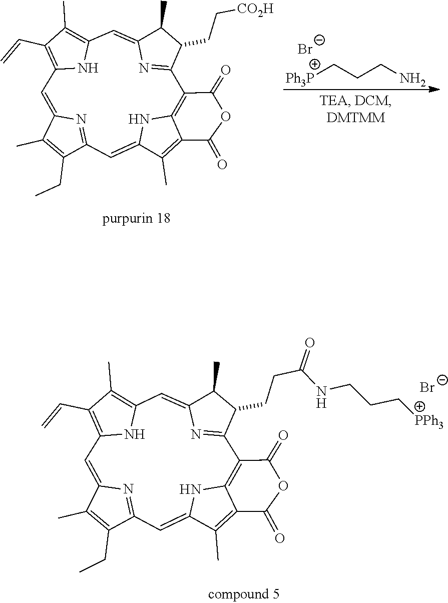

To a 25 mL RBF was added purpurin 18 (200 mg, 0.354 mmol, 1 eq), 4-(4,6-dimethoxy-1,3,5-triazin-2-yl)-4-methylmorpholinium chloride (DMTMM) (137 mg, 0.496 mmol, 1.4 eq), DCM (10 mL), triethylamine (49 μL, 0.354 mmol, 1 eq) and (3-aminopropyl)triphenylphosphonium bromide (199 mg, 0.496 mmol, 1.4 eq). The resultant mixture was stirred under nitrogen at 23 °C for 30 minutes. The reaction mixture was diluted with DCM (10 mL), washed sequentially with 0.5 M HCl (10 mL) and pH=7 phosphate buffer before being dried (Na₂SO₄) and concentrated using a rotary evaporator to give the crude residue as a dark red solid. The residue was subjected to column chromatography eluting using a gradient of 5-10% MeOH/DCM. Fractions containing the product (major red spot, R_{f}=0.15 in 7% MeOH/DCM) were combined to give compound 5 as a red powder (284 mg, 85%).

¹H NMR (400 MHz, CDCl₃) δ 9.49 (s, 1H), 9.23 (s, 1H), 9.19 (t, 1H), 8.63 (s, 1H), 7.87-7.79 (m, 7H), 7.74-7.64 (m, 10H), 6.36 (d, 1H), 6.14 (d, 1H), 5.22 (d, 1H), 4.62 (q, 1H), 4.30 (m, 1H), 4.03-3.90 (m, 1H), 3.88-3.76 (m, 1H), 3.72 (s, 3H), 3.60-3.51 (m, 3H), 3.49 (m, 2H), 3.31 (s, 3H), 3.10 (s, 3H), 2.86-2.80 (m, 1H), 2.62-2.50 (m, 2H), 1.98-1.86 (m, 2H), 1.80-1.70 (m, 10H), 1.62 (t, 3H), 0.35 (brs, 1H), 0.03 (brs, 1H).

### Synthesis Example 6—Synthesis of purpurin 18 (N-(3-triphenylphosphoniumpropyl)bromide)amide N-(2-(2-methoxyethoxy)ethyl)imide (compound 6)

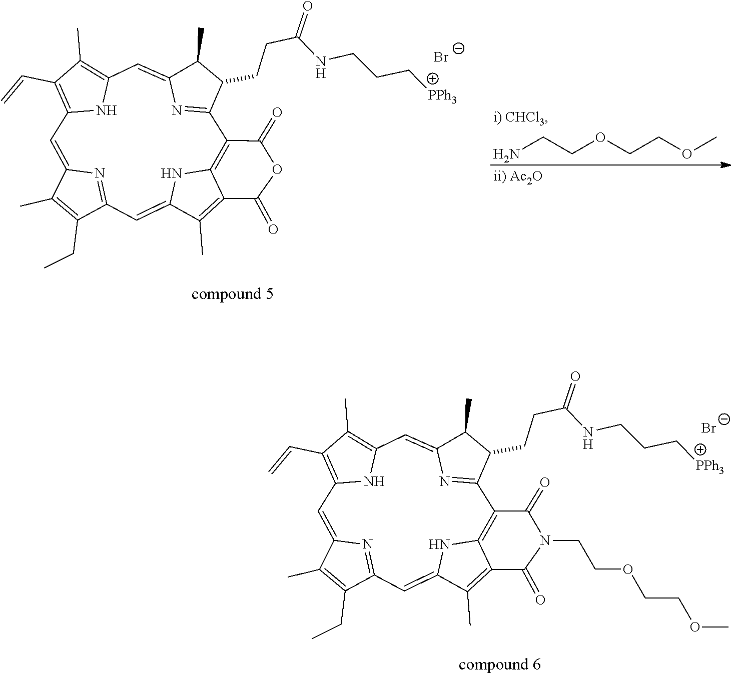

To a 25 mL RBF was added compound 5 (50 mg, 0.0528 mmol, 1 eq), 2-(methoxyethoxy)ethanamine (31 mg, 0.264 mmol, 5 eq) and chloroform (3 mL). The resultant mixture was stirred under nitrogen at 25 °C. After 3 hours, further 2-(methoxyethoxy)ethanamine (31 mg, 0.264 mmol, 5 eq) was added and stirring continued overnight at 25 °C. The reaction mixture was concentrated by rotary evaporation and to the resulting residue was added acetic anhydride (1 mL) and the mixture was stirred with heating at 70 °C for 2 hours. The solution was cooled and 5% NaHCO₃ (3 mL) was added and the mixture stirred for 2 hours. Water (10 mL) was added and the mixture was extracted with DCM (3×5 mL). The combined organics were dried (Na₂SO₄) and concentrated by rotary evaporation to give the crude imide as a red residue (~120 mg). The residue was purified by column chromatography using 5-10% MeOH/DCM, loaded as a solution in the eluent. Fractions with a major red spot at R_{f}=0.4 in 8% MeOH/DCM were collected and concentrated to give compound 6 as a dark red solid (28 mg, 51% yield).

¹H NMR (400 MHz, CDCl₃) δ 9.58 (s, 1H), 9.29 (s, 1H), 9.01 (brs, 1H), 8.70 (s, 1H), 7.90-7.78 (m, 2H), 7.71-7.50 (m, 20H), 6.26 (d, 1H), 6.11 (d, 1H), 5.31 (d, 1H), 4.72-4.65 (m, 3H), 4.35-4.27 (m, 2H), 4.07-4.00 (m, 2H), 3.86-3.81 (m, 2H), 3.81 (s, 4H), 3.65-3.59 (m, 5H), 3.38-3.30 (m, 8H), 3.12 (s, 3H), 2.85-2.75 (m, 1H), 2.60-2.49 (m, 2H), 1.95-1.70 (m, 14H), 1.63 (t, 5H), 0.01 (brs, 1H), −0.12 (brs, 1H).

### Synthesis Example 7—Synthesis of purpurin 18 N-hexylimide 13-(N-methyl-5-triphenylphosphonium bromide pentanamide) methyl ester (compound 7)

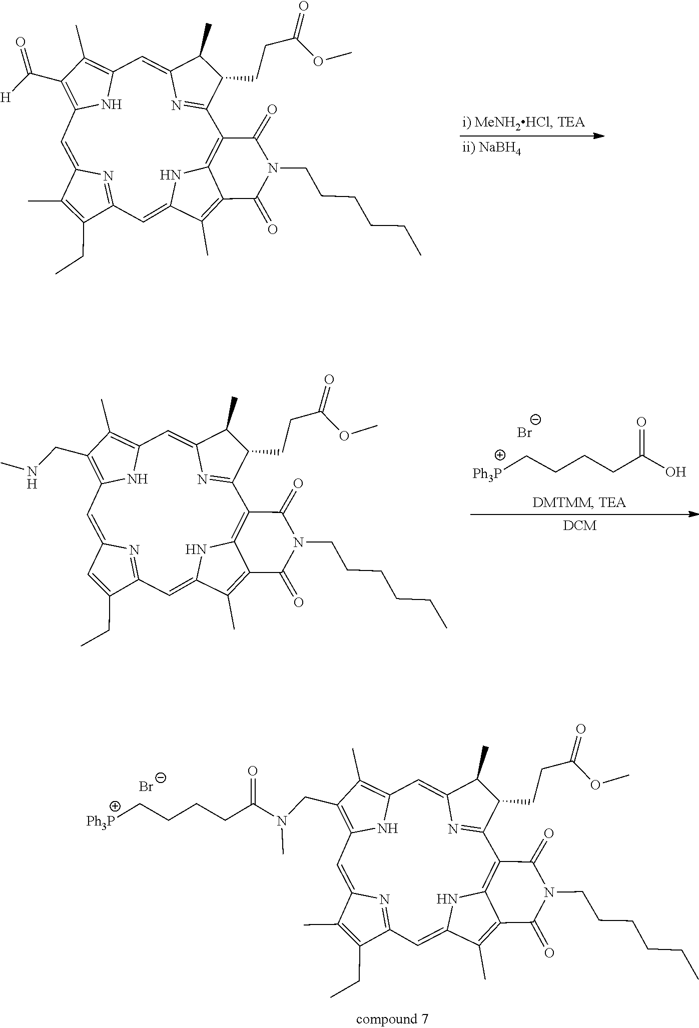

Step 1: To a 250 mL RBF was added purpurin 18 13-formyl N-hexylimide methyl ester (333 mg, 0.502 mmol, 1 eq), DCM (7 mL), methanol (28 mL), TEA (253 mg, 2.51 mmol, 5 eq) and methylamine hydrochloride (169 mg, 2.51 mmol, 5 eq). The resultant mixture was stirred (400 rpm) under nitrogen in the dark for 4 hours. NaBH₄ (190 mg, 5.02 mmol, 10 eq) was added and stirring was continued for 16 hours. The reaction progress was monitored by TLC. The reaction was acidified with 2 M HCl (2 mL) and stirred for 10 minutes. Phosphate buffer pH=7 (20 mL) was added and the mixture extracted with DCM (50 mL) before being dried (Na₂SO₄) and concentrated by rotary evaporation to give a dark red residue. The residue was purified by column chromatography using 5-7% MeOH/DCM, loaded as a solution in the eluent. Fractions containing a dark red band (R_{f}=0.30 in 5% MeOH/DCM) were combined and concentrated by rotary evaporation to give purpurin 18 N-hexylimide 13-N-methylamino methyl ester as a dark blue solid (110 mg, 32%). (97.40% purity by HPLC).

¹H NMR (400 MHz, Chloroform-d) δ 9.49 (s, 1H), 9.29 (s, 1H), 8.48 (s, 1H), 5.32 (dd, J=8.8, 2.8 Hz, 1H), 4.78 (s, 2H), 4.37 (td, J=9.1, 2.6 Hz, 2H), 4.25 (dq, J=13.0, 6.9 Hz, 1H), 3.70 (s, 3H), 3.55 (q, J=7.3, 5.9 Hz, 2H), 3.49 (s, 3H), 3.24 (s, 3H), 3.10 (s, 3H), 2.65 (s, 3H), 2.63-2.56 (m, 1H), 2.41-2.21 (m, 2H), 1.98-1.84 (m, 4H), 1.68 (d, J=7.3 Hz, 3H), 1.54 (dt, J=19.3, 7.5 Hz, 5H), 1.44-1.29 (m, 3H), 0.87 (t, J=7.1 Hz, 3H), 0.83-0.69 (m, 1H), −0.29 (s, 1H), −0.34 (s, 1H).

Step 2: To a 10 mL RBF was added purpurin 18 N-hexylimide 13-N-methylamino methyl ester (100 mg, 0.147 mmol, 1 eq), 4-(carboxybutyl)triphenylphosphonium bromide (130 mg, 0.294 mmol, 2 eq), triethylamine (45 mg, 441 mmol, 3 eq), DCM (3 mL) and DMTMM (87 mg, 0.294 mmol, 2 eq). The resultant mixture was stirred (400 rpm) under nitrogen at 25 °C in the dark for 18 hours. The reaction progress was monitored by HPLC. The reaction mixture was transferred to a separatory funnel, diluted with DCM (30 mL) and washed with 0.5 M HCl (20 mL). The organic layer was then washed with pH 7 phosphate buffer (20 mL) followed by 1 M NaHCO₃ (20 mL). The organic phase was dried (Na₂SO₄) and concentrated by rotary evaporation to give a blue-black residue (120 mg). The residue was then subjected to column chromatography. The crude product was dissolved in 5% MeOH/DCM and eluted using a gradient of 5-10% MeOH/DCM. Fraction size of 25 mL were collected when first colour began to elute. Fractions containing the product (Major dark red spot, R_{f}=0.3 in 5% MeOH/DCM) were combined to give compound 7 as a dark blue solid (96 mg, 59%). (95.72% purity by HPLC).

¹H NMR (400 MHz, Chloroform-d) δ 9.64 (s, 1H), 9.60 (s, 1H), 8.56 (s, 1H), 7.93-7.75 (m, 5H), 7.75-7.50 (m, 8H), 5.75 (s, 2H), 5.40 (dd, J=9.0, 2.8 Hz, 1H), 4.49-4.40 (m, 2H), 4.35 (q, J=7.2 Hz, 1H), 3.97-3.86 (m, 1H), 3.82 (s, 3H), 3.62 (q, J=7.5 Hz, 2H), 3.55 (s, 3H), 3.47 (s, 1H), 3.32 (s, 3H), 3.21 (s, 3H), 3.10 (s, 3H), 2.83 (t, J=7.1 Hz, 2H), 2.68 (ddd, J=15.5, 10.6, 5.3 Hz, 1H), 2.48-2.38 (m, 1H), 2.32 (ddd, J=15.2, 10.8, 4.4 Hz, 1H), 2.15 (h, J=7.0, 6.3 Hz, 2H), 2.06-1.92 (m, 2H), 1.85 (q, J=8.3, 7.9 Hz, 3H), 1.76 (d, J=7.3 Hz, 5H), 1.62 (dt, J=13.4, 7.6 Hz, 5H), 1.51-1.36 (m, 3H), 0.94 (t, J=7.1 Hz, 3H), 0.90-0.74 (m, 2H), −0.14 (s, 1H), −0.25 (s, 1H).

### Synthesis Example 8—Synthesis of purpurin 18 N-hexylimide 13-(N-3-triphenylphosphonium bromide)aminomethyl methyl ester (compound 8)

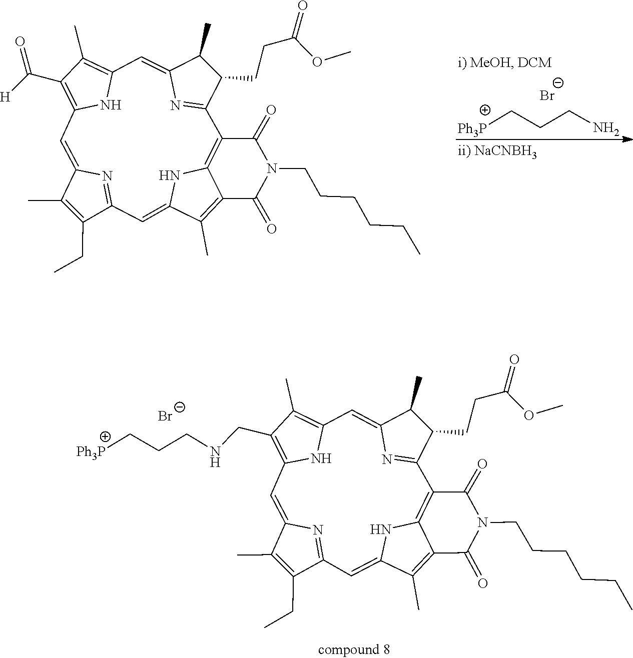

To a 50 mL RBF was added purpurin 18 13-formyl N-hexylimide methyl ester (190 mg, 0.286 mmol, 1 eq), (3-aminopropyl)triphenylphosphonium bromide (343 mg, 0.858 mmol, 3 eq), 4 Å molecular sieves (400 mg), MeOH (12 mL) and DCM (4 mL). The resultant mixture was stirred (400 rpm) under nitrogen at 25 °C in the dark for 2 hours. A further portion of (3-aminopropyl) triphenylphosphonium bromide (226 mg, 0.572 mmol, 2 eq) was added and the reaction stirred for a further 2 hours. Sodium cyanoborohydride (176 mg, 2.86 mmol, 10 eq) was then added and the reaction stirred for 3 days. The reaction progress was monitored by TLC. Water (20 mL) was then added and the reaction stirred for 10 minutes. The resulting mixture was then extracted with DCM (3×20 mL) and the combined organic phases washed with saturated NaHCO₃ solution (2×30 mL). The organic layer was then dried over Na₂SO₄, filtered on a sintered glass funnel and concentrated by rotary evaporation to give the crude product as a dark red solid. The crude product was subjected to column chromatography (3×25 cm). The crude product was dissolved in 3% MeOH/DCM and eluted using the same solvent. Fractions containing the product (Major dark red spot, R_{f}=0.3 in 5% MeOH/DCM) were combined to give compound 8 as a dark blue solid (82 mg, 27%). (84.61% purity by HPLC).

¹H NMR (400 MHz, Chloroform-d) δ 9.55 (d, J=7.8 Hz, 2H), 8.53 (s, 1H), 7.85-7.68 (m, 2H), 7.57-7.46 (m, 3H), 7.45-7.32 (m, 11H), 5.39 (dd, J=8.9, 2.6 Hz, 1H), 4.96 (s, 2H), 4.50-4.40 (m, 2H), 4.40-4.26 (m, 1H), 3.81 (s, 3H), 3.66-3.52 (m, 5H), 3.39 (ddd, J=12.7, 8.4, 5.1 Hz, 2H), 3.29 (s, 3H), 3.17 (t, J=6.3 Hz, 2H), 3.07 (s, 3H), 2.77-2.64 (m, 1H), 2.51-2.29 (m, 2H), 1.98 (p, J=5.9 Hz, 3H), 1.78 (dd, J=16.3, 7.3 Hz, 4H), 1.60 (q, J=7.2 Hz, 5H), 1.54-1.36 (m, 4H), 1.27 (q, J=13.3, 11.3 Hz, 2H), 0.94 (t, J=7.1 Hz, 3H), 0.90-0.70 (m, 7H), −0.16 (s, 1H), −0.31 (s, 1H).

### Synthesis Example 9—Synthesis of purpurin 18 N-hexylimide 13-(N-3-triphenylphosphonium bromide ethylcarbamate)aminomethyl methyl ester (compound 9)

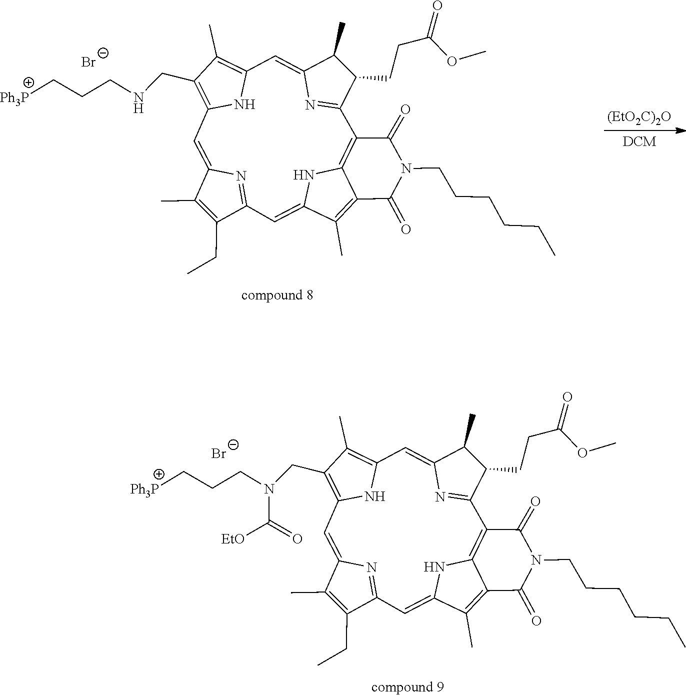

To a 10 mL RBF was added compound 8 (72 mg, 0.0687 mmol, 1 eq), diethyl dicarbonate (56 mg, 0.344 mmol, 5 eq) and DCM (3 mL). The resultant mixture was stirred (400 rpm) under nitrogen at 25 °C in the dark for 1 hour. The reaction progress was monitored by HPLC. The reaction mixture was then concentrated by rotary evaporation to give the crude product as a dark red residue. The crude product was subjected to column chromatography (3×25 cm). The crude product was dissolved in 2% MeOH/DCM and eluted using the same solvent. Fractions containing the product (Major dark red spot, R_{f}=0.3 in 5% MeOH/DCM) were combined to give compound 9 as a dark blue solid (41 mg, 53%). (95.96% purity by HPLC).

¹H NMR (400 MHz, Chloroform-d) δ 9.62 (s, 2H), 8.65 (s, 1H), 7.71 (d, J=4.9 Hz, 1H), 7.58-7.48 (m, 1H), 7.05-6.81 (m, 7H), 6.74 (s, 8H), 5.73 (t, J=15.7 Hz, 2H), 5.44-5.35 (m, 1H), 4.53-4.44 (m, 2H), 4.40 (q, J=7.5 Hz, 1H), 4.30 (dd, J=11.3, 5.0 Hz, 2H), 3.86 (s, 5H), 3.67 (q, J=7.6 Hz, 2H), 3.60 (s, 3H), 3.44 (s, 3H), 3.14 (s, 3H), 2.89-2.75 (m, 1H), 2.49 (t, J=14.1 Hz, 2H), 2.08-1.85 (m, 3H), 1.80 (d, J=7.3 Hz, 3H), 1.67 (t, J=7.6 Hz, 5H), 1.63-1.54 (m, 8H), 1.52-1.37 (m, 9H), 1.34 (s, 1H), 1.33-1.05 (m, 13H), 0.95 (t, J=7.1 Hz, 3H), 0.90-0.72 (m, 11H), 0.45 (s, 1H), 0.24 (s, 1H), −0.18-−0.29 (m, 1H), −0.48 (s, 1H).

### Synthesis Example 10—Synthesis of meso-purpurin 18 (N-(3-triphenylphosphoniumpropyl)bromide)amide (compound 10)

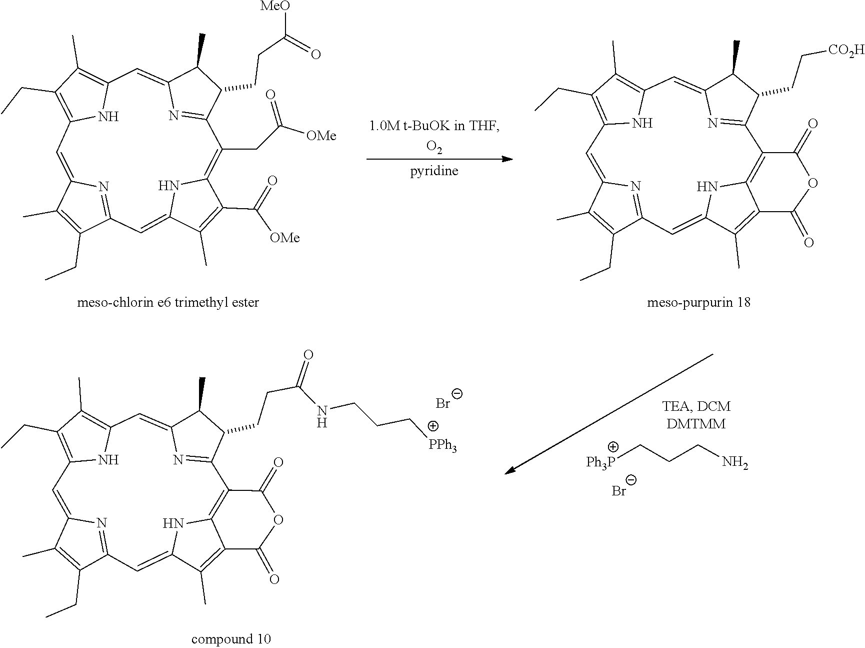

Step 1: To a 3N 100 mL RBF charged with a stir bar and fitted with an air condenser connected to a bubbler and a sparging tube was added meso-chlorin e6 trimethyl ester (1.00 g, 1.566 mmol, 1 eq) and pyridine (30 mL). The sparging tube was connected to an aquarium pump using tubing that incorporated a bleed valve, and air was streamed vigorously into the reaction solution via the sparging tube. The reaction mixture was then heated to 50 °C in an oil bath with stirring for 10 minutes with air continuing to be pumped into the reaction solution. 1.0 M Potassium tert-butoxide (16 mL, 14.1 mmol, 9 eq) was then added and the reaction then stirred at 25 °C for 90 minutes. The reaction was then quenched with acetic acid (25 mL) and the resulting solution poured into water (100 mL). The aqueous mixture was extracted with DCM and then the combined organic layers were washed with 2M HCl then water. The organic phase with then dried over Na₂SO₄, filtered on a sintered glass funnel and concentrated by rotary evaporation to give the crude product as a dark purple solid (1.3 g). The crude product was purified by column chromatography. The crude product was dissolved in 2% MeOH/DCM and eluted using the same solvent. Fractions of 20 mL were collected when the main dark band began to elute and fractions containing the product (R_{f}=0.4 in 5% MeOH/DCM) were combined and concentrated to give meso-purpurin 18 as a dark purple solid (310 mg, 35%).

¹H NMR (400 MHz, CDCl₃) δ 9.37 (s, 1H), 9.14 (s, 1H), 8.46 (s, 1H), 5.12-5.08 (m, 1H), 4.32 (q, 1H), 3.72 (q, 2H), 3.62 (s, 3H), 3.56 (q, 2H), 3.21 (s, 3H), 3.13 (s, 3H), 2.80-2.70 (m, 1H), 2.51-2.34 (m, 2H), 1.92-1.84 (m, 1H), 1.72-1.66 (m, 6H), 1.61 (t, 3H), 0.13 (brs, 1H), −0.18 (brs, 1H).

Step 2: To a 25 mL RBF was added meso-purpurin 18 (100 mg, 0.176 mmol, 1 eq), DMTMM (68 mg, 0.247 mmol, 1.4 eq), DCM (5 mL), triethylamine (24 μL, 0.176 mmol, 1 eq) and (3-aminopropyl)triphenylphosphonium bromide (99 mg, 0.247 mmol, 1.4 eq). The resultant mixture was stirred under nitrogen at 22 °C for 30 minutes. The reaction mixture was diluted with DCM (10 mL), washed sequentially with 0.5 M HCl and pH=7 phosphate buffer before being dried (Na₂SO₄) and concentrated using a rotary evaporator to give the crude residue as a dark purple solid. The residue was subjected to column chromatography eluting using a gradient of 5-9% MeOH/DCM. Fractions containing the product (R_{f}=0.15 in 7% MeOH/DCM) were combined to give compound 10 as a purple powder (147 mg, 88%).

¹H NMR (400 MHz, CDCl₃) δ 9.49 (s, 1H), 9.10 (s, 1H), 9.03 (t, 1H), 8.57 (s, 1H), 7.87-7.79 (m, 6H), 7.74-7.63 (m, 9H), 5.22 (d, 1H), 4.62 (q, 1H), 4.01-3.88 (m, 1H), 3.87-3.78 (m, 1H), 3.75-3.67 (m, 5H), 3.63-3.52 (m, 3H), 3.51-3.44 (m, 1H), 3.22 (s, 3H), 3.13 (s, 3H), 2.86-2.76 (m, 1H), 2.62-2.47 (m, 2H), 2.02-1.87 (m, 2H), 1.80-1.76 (m, 4H), 1.73-1.61 (m, 10H), 0.48 (brs, 1H), 0.08 (brs, 1H).

### Synthesis Example 11—Synthesis of meso-purpurin 18 (N-(3-triphenylphosphoniumpropyl)bromide)amide N-(2-(2-methoxyethoxy)ethyl)imide (compound 11)

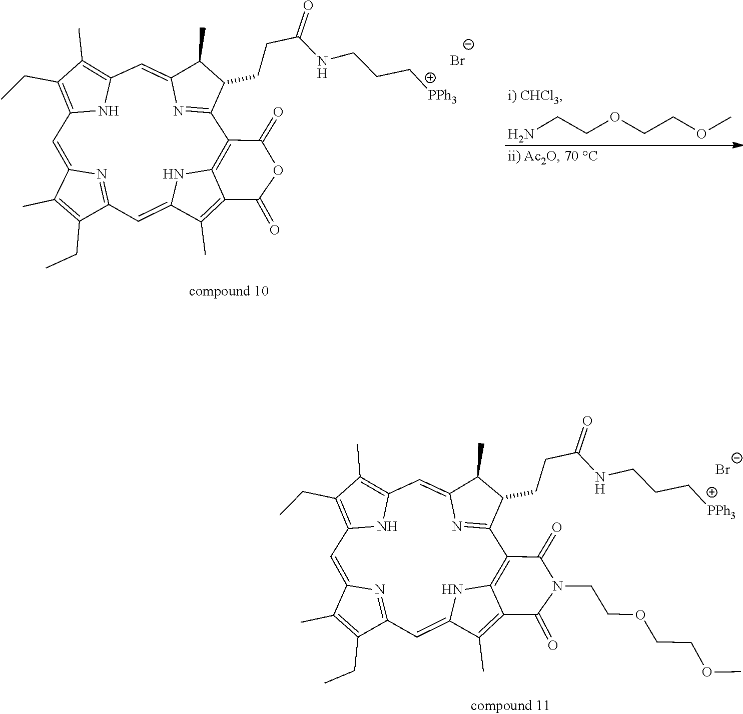

To a 25 mL RBF was added compound 10 (50 mg, 0.0527 mmol, 1 eq), 2-(methoxyethoxy)ethanamine (63 mg, 0.527 mmol, 10 eq) and chloroform (3 mL). The resultant mixture was stirred under nitrogen at 25 °C. After 3 hours, further 2-(methoxyethoxy)ethanamine (63 mg, 0.527 mmol, 10 eq) was added and stirring continued overnight at 25 °C. The reaction mixture was concentrated by rotary evaporation and to the resulting residue was added acetic anhydride (1 mL) and the mixture was stirred with heating at 70 °C for 1 hour. The solution was cooled and saturated NaHCO₃ (3 mL) was added and the mixture stirred for 1 hour. Water (10 mL) was added and the mixture was extracted with DCM. The organic layer was dried (Na₂SO₄) and concentrated by rotary evaporation to give the crude imide as a purple residue. The residue was purified by column chromatography using 5-10% MeOH/DCM, loaded as a solution in the eluent. Fractions with a major purple spot at R_{f}=0.4 in 8% MeOH/DCM were collected and concentrated to give compound 11 as a dark purple solid (30 mg, 55%).

¹H NMR (400 MHz, CDCl₃) δ 9.53 (s, 1H), 9.10 (s, 1H), 9.04 (brs, 1H), 8.60 (s, 1H), 7.85-7.78 (m, 2H), 7.75-7.50 (m, 20H), 5.29 (d, 1H), 4.71-4.58 (m, 3H), 4.08-4.00 (m, 2H), 3.90-3.80 (m, 3H), 3.79 (m, 4H), 3.70-3.65 (m, 5H), 3.63-3.58 (m, 5H), 3.35 (s, 3H), 3.1221 (s, 3H), 3.14 (s, 3H), 2.88-2.78 (m, 1H), 2.60-2.49 (m, 2H), 2.05-1.90 (m, 7H), 1.80-1.60 (m, 20H), 0.16 (brs, 1H), −0.06 (brs, 1H).

### Synthesis Example 12—Synthesis of purpurin 18 N-(3-triphenylphosphoniumpropylbromide)imide N-methyl-N-3,6,9,12,15,18-hexaoxanonadecyl propylamide (compound 12)

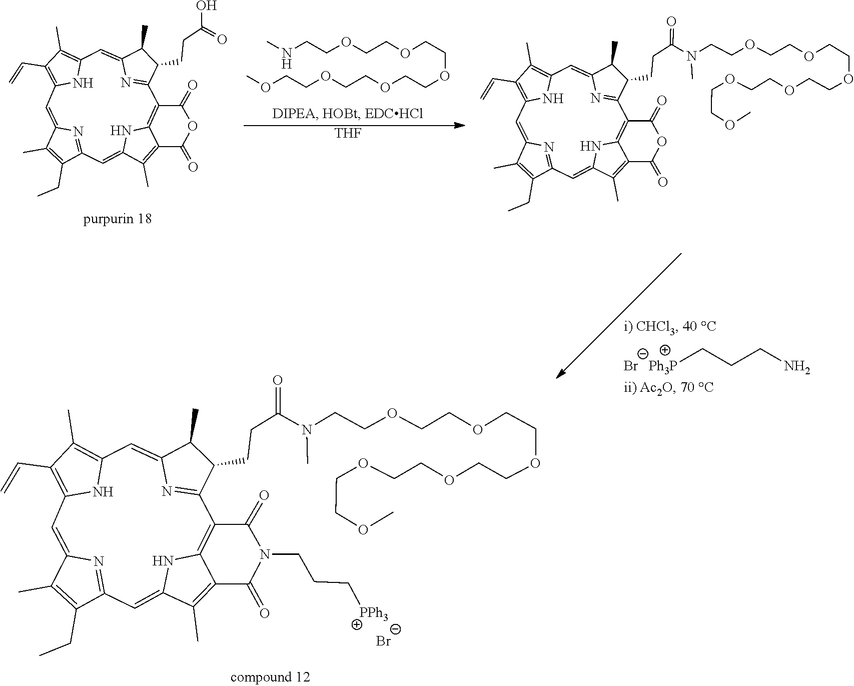

Step 1: A 1N 25 mL RBF was charged with purpurin 18 (300 mg, 0.531 mmol, 1 eq), EDC.HCl (153 mg, 0.797 mmol, 1.5 eq), DIPEA (137 mg, 1.06 mmol, 2 eq), HOBt (81 mg, 0.531 mmol, 1 eq) and THF (12 mL). The mixture was stirred under nitrogen at 30 °C for 16 hours, then N-methyl-2,5,8,11,14,17-hexaoxanonadecan-19-amine (197 mg, 0.637 mmol, 1.2 eq) was added. The resultant solution was stirred for 6 hours under a nitrogen atmosphere at 30 °C. The reaction progress was monitored by HPLC. The reaction mixture was then concentrated by rotary evaporation and re-dissolved in DCM (50 mL). The resultant solution was transferred to a separating funnel and washed with 0.5M HCl (2×50 mL) then pH7 buffer solution (30 mL). The organic layer was then dried over Na₂SO₄, filtered on a sintered glass funnel and concentrated by rotary evaporation to give the crude product as a dark blue solid. The crude product was then purified by column chromatography. An eluent gradient of 2-5% MeOH in DCM was used to elute the product band from the column. Fractions containing a red-brown spot by TLC analysis at R_{f}=0.3 in 5% MeOH/DCM were combined and concentrated by rotary evaporation to give purpurin 18 N-methyl-N-3,6,9,12,15,18-hexaoxanonadecyl propylamide as a dark blue solid (283 mg, 62%). (99.32% purity by HPLC).

¹H NMR (400 MHz, Chloroform-d) δ 9.40 (s, 1H), 9.24 (d, J=1.7 Hz, 1H), 8.55 (d, J=1.5 Hz, 1H), 7.83 (dd, J=17.8, 11.5 Hz, 1H), 6.27 (dd, J=17.8, 1.3 Hz, 1H), 6.16 (dd, J=11.6, 1.3 Hz, 1H), 5.16-5.04 (m, 1H), 4.49 (q, J=7.2 Hz, 1H), 3.66 (s, 3H), 3.65-3.47 (m, 22H), 3.36 (s, 3H), 3.34 (s, 3H), 3.12 (s, 2H), 3.07 (s, 3H), 2.98 (dt, J=9.4, 4.8 Hz, 1H), 2.93 (s, 1H), 2.77-2.61 (m, 1H), 2.46-2.33 (m, 1H), 2.03-1.91 (m, 1H), 1.79-1.70 (m, 5H), 1.59 (t, J=7.6 Hz, 3H), 0.19 (s, 1H), −0.10 (s, 1H).

Step 2: To a 25 mL RBF was added purpurin 18 N-methyl-N-3,6,9,12,15,18-hexaoxanonadecyl propylamide (400 mg, 0.467 mmol, 1 eq), (3-aminopropyl)triphenylphosphonium bromide (937 mg, 2.34 mmol, 5 eq) and chloroform (15 mL). The resultant mixture was stirred under nitrogen at 40 °C for 16 hours. The reaction progress was monitored by TLC. The reaction mixture was concentrated by rotary evaporation and to the resulting residue was added acetic anhydride (5 mL) and the mixture was stirred with heating at 70 °C for 2 hours. The reaction progress was monitored by HPLC. The solution was cooled and 5% NaHCO₃ (15 mL) was added and the mixture stirred for 2 hours. Water (30 mL) was added and the mixture was extracted with DCM. The organic layer was dried (Na₂SO₄) and concentrated by rotary evaporation to give the crude imide as a red residue. The residue was purified by column chromatography using 4-8% MeOH/DCM, loaded as a solution in the eluent. Fractions with a major red spot at R_{f}=0.3 in 5% MeOH/DCM were collected and concentrated to give compound 12 as a dark blue solid (230 mg, 40%). (97.41% purity by HPLC).

¹H NMR (400 MHz, Chloroform-d) δ 9.39 (d, J=3.5 Hz, 1H), 9.28 (s, 1H), 8.56 (d, J=2.5 Hz, 1H), 7.92-7.79 (m, 7H), 7.70-7.57 (m, 10H), 6.29 (dd, J=17.8, 1.3 Hz, 1H), 6.16 (dd, J=11.6, 1.3 Hz, 1H), 5.26-5.14 (m, 1H), 4.72-4.62 (m, 2H), 4.39 (t, J=7.1 Hz, 1H), 4.04 (dd, J=15.9, 13.0 Hz, OH), 3.64 (d, J=3.0 Hz, 3H), 3.62-3.57 (m, 6H), 3.57-3.49 (m, 7H), 3.49-3.42 (m, 2H), 3.39-3.31 (m, 8H), 3.28-3.18 (m, 2H), 3.17-3.08 (m, 5H), 2.65 (s, 2H), 2.49 (s, 1H), 2.42 (dq, J=15.4, 9.3, 8.4 Hz, 3H), 2.27 (s, 1H), 2.11-1.96 (m, 1H), 1.76 (t, J=6.7 Hz, 3H), 1.61 (td, J=7.6, 2.2 Hz, 3H), −0.05 (d, J=7.9 Hz, 2H).

### Synthesis Example 13—Synthesis of purpurin 18 N-hydroxylimide (N-(3-triphenylphosphoniumpropyl)bromide)amide (compound 13)

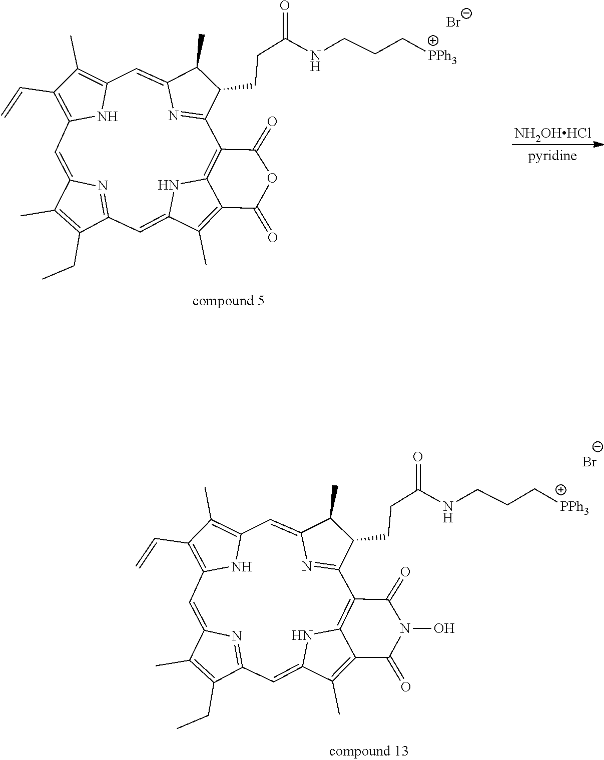

To a 50 mL RBF was added compound 5 (100 mg, 0.106 mmol, 1 eq), hydroxylamine hydrochloride (74 mg, 1.06 mmol, 10 eq) and pyridine (15 mL). The resultant mixture was stirred under nitrogen at 25 °C for 18 hours. The reaction progress was monitored by HPLC. The reaction mixture was then diluted with DCM (30 mL) and washed with 0.2 M HCl. The organic layer was then washed with water and dried over Na₂SO₄. The dried organic layer was decanted from the Na₂SO₄ and concentrated by rotary evaporation to give compound 13 as a dark blue solid (99 mg, 97%). (97.23% purity by HPLC).

¹H NMR (400 MHz, Chloroform-d) δ 9.19 (s, 1H), 9.09 (s, 2H), 8.47 (s, 1H), 7.78-7.62 (m, 7H), 7.60-7.43 (m, 9H), 6.18-6.08 (m, 1H), 6.00 (dd, J=11.5, 1-3 Hz, 1H), 5.23 (m, 2H), 4.44 (q, J=7.1 Hz, 1H), 4.33-4.12 (m, 1H), 4.02-3.75 (m, 2H), 3.50 (d, J=5.7 Hz, 2H), 3.44 (d, J=9.0 Hz, 5H), 3.21 (s, 3H), 3.00 (s, 3H), 2.76 (dt, J=15.3, 7.9 Hz, 1H), 2.60 (ddd, J=14.2, 8.8, 5.3 Hz, 1H), 2.39 (d, J=8.7 Hz, 1H), 1.85 (d, J=18.1 Hz, 6H), 1.70-1.64 (m, 3H), 1.51 (t, J=7.6 Hz, 3H), 0.16 (s, 1H), 0.10 (s, 1H).

### BIOLOGICAL EXPERIMENTAL DETAILS

### Example 1—Cytotoxicity, Phototoxicity and Therapeutic Index

### Preparation of Photosensitizer Stock Solutions

Photosensitizers (e.g. purpurin analogue, chlorin e4 disodium (provided by Advanced Molecular Technologies, Scoresby) or Talaporfin sodium (purchased from Focus Bioscience cat #HY-16477-5MG)) were resuspended in 100% dimethylsulfoxide (DMSO) at a concentration of 5.5 mM. Samples were stored at 4 °C protected from light.

### Preparation of Photosensitizers for In Vitro Studies

For in vitro experiments, photosensitizers (stock solution 5.5 mM in 100% DMSO) were diluted 1:100 in concentrated excipient solution (final 55 μM photosensitizer in 10% w/v Kollidon-12, 42.4% w/v polysorbate 80, 40% w/v ethanol, 1.0% DMSO). Serial dilutions were prepared in cell culture media (Dulbecco's Modified Eagle Medium/Nutrient Mixture F-12 (DMEM/F-12)) supplemented with 10% v/v Fetal Bovine Serum, 100 U/mL penicillin, 100 μg/mL streptomycin and the same excipient solution at a constant 1:55 dilution.

### Cell Culture

Human ovarian cancer cell line SKOV3 (ATCC #HTB-77) was maintained in Dulbecco's Modified Eagle Medium/Nutrient Mixture F-12 (DMEM/F-12), supplemented with % v/v Fetal Bovine Serum, 100 U/mL penicillin and 100 μg/mL streptomycin. Monolayer cultures were grown in a humidified incubator at 37 °C with 5% CO₂. Once cells had reached ~80% confluence, spent media was replaced with media containing photosensitizer at the required concentration and cells were incubated for the desired period of time to allow photosensitizer uptake.

### Statistical Analyses

All data were analysed using GraphPad PRISM v8.3.1 (549) (GraphPad Software, CA). Spectral absorbance and viability measurements were normalized in the range 0-100%, with a minimum of 0 and a maximum value determined from the dataset. Dose response was determined using a sigmoidal four-point non-linear regression with variable slope, and IC10 or IC90 calculated for each compound. All data are shown as mean±SD (where appropriate).

### Cytotoxicity

SKOV3 cells were seeded in 96-well black wall plates (Greiner #655090) at a cell density of 5000 cells in 100 μl culture medium per well. On reaching ~60% confluence, media was aspirated and replaced with fresh media containing the relevant purpurin analogue from 0-100 μM in DMSO. Cells were incubated for a further 24 hours, allowing uptake of purpurin analogues.

To test for inherent cytotoxicity (i.e. “dark toxicity”) of the purpurin analogues, the culture media was replaced after 24 hours with fresh media containing 10% (v/v) AlamarBlue Cell Viability Reagent (ThermoFisher) and cells incubated at 37 °C for 6 hours. Untreated cells were used as a control. Fluorescence (Ex 555 nm/Em 596 nm) was measured using a CLARIOstar Plus Microplate Readear (BMG Labtech), and cytotoxicity assessed according to the % viable cells remaining. All measurements were made in quadruplicate.

### Phototoxicity

SKOV3 cells were seeded in 96-well black wall plates (Greiner #655090) at a cell density of 5000 cells in 100 μl culture medium per well. On reaching ~60% confluence, media was aspirated and replaced with fresh media containing the relevant purpurin analogue from 0-100 μM in DMSO. Cells were incubated for a further 24 hours, allowing uptake of purpurin analogues.

To test for phototoxicity, cells incubated with purpurin analogues (0-20 M in DMSO) had culture media replaced after 24 hours (as above) and were then exposed to a 680 nm light-emitting diode (LED) panel (Invion) with optical power density at 50 mW/cm² for 5 mins (total 15 J/cm²). Laser and LED exposure induce an equivalent response in relation to phototoxicity. Following activation, cells were cultured for a further 24 hours. Media was then replaced with fresh media containing AlamarBlue, and % viable cells remaining assessed as above. Controls included cells treated with purpurin analogues but not activated by laser light; cells without purpurin analogue treatment but with laser light; and untreated controls. All measurements were made in quadruplicate.

### Toxicity Profile for Purpurin Analogues

The phototoxicity and inherent cytotoxicity (i.e. “dark toxicity”) of purpurin analogues were assessed as previously using SKOV3 ovarian cancer cells. For comparative purposes, purpurin analogues were compared against chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+(IC90/X)\hat{ }HillSlope (phototoxicity IC90)) or Y=100/(1+(IC10/X)\hat{ }HillSlope (dark toxicity IC10)).

Phototoxicity and dark toxicity values are provided in Table 1. The purpurin analogues had a substantially better IC90 than chlorin e4 disodium (IC90 21.32 μM) or Talaporfin sodium (IC90 22.83 μM). Thus, purpurin analogues achieved an up to ~4,500-fold increase in phototoxicity compared to Talaporfin sodium, a clinically approved photosensitizer.

Some variation in the dark toxicity of the purpurin analogues of the present invention was observed (Table 1). The greater phototoxicity afforded by the purpurin analogues of the present invention, however, is expected to offset any dark toxicity issue through a decreased dose requirement in use.

### Therapeutic Index for Purpurin Analogues

To evaluate the therapeutic potential of purpurin analogues, the therapeutic index (TI) was calculated. TI provides a quantitative measurement to describe relative drug safety, by comparing the drug concentration required for desirable effects versus the concentration resulting in undesirable off-target toxicity. TI was calculated using phototoxicity IC90 vs dark toxicity IC10.

TI values are provided in Table 1. Talaporfin sodium had a low therapeutic index (TI=0.49) with chlorin e4 disodium only marginally better (TI=1.89), indicating that whilst their relative cytotoxicity is low, the potential therapeutic window for their use is small. The purpurin analogues of the present invention had comparatively significantly improved TIs with substantially greater phototoxicity (Table 1).

Thus, the purpurin analogues of the present invention have a desirable therapeutic index that is better than a clinically applied photosensitizer. Moreover, the greater phototoxicity of the purpurin analogues suggests their potential use at a greatly reduced dose in vivo. The purpurin analogues therefore have an acceptable therapeutic profile for clinical application.

TABLE 1
Toxicity profile and therapeutic index for purpurin analogues:

| | Phototoxicity | Dark toxicity | Therapeutic |
|---|---|---|---|
| Species | μM (IC90) | μM (IC10) | Index |
| Compound 1 | 0.046 | 79.63 | 1731.09 |
| Compound 2 | 0.027 | 77.63 | 2875.19 |
| Compound 3 | 0.102 | 50.84 | 498.43 |
| Compound 4 | 0.010 | 49.91 | 4991.00 |
| Compound 5 | 0.005 | 49.88 | 9976.00 |
| Compound 6 | 0.009 | 51.07 | 5674.44 |
| Compound 7 | 0.021 | 49.40 | 2352.38 |
| Chlorin e4 disodium | 21.32 | 40.23 | 1.89 |
| Talaporfin sodium | 22.83 | 11.10 | 0.49 |

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I) or a complex of formula (II): 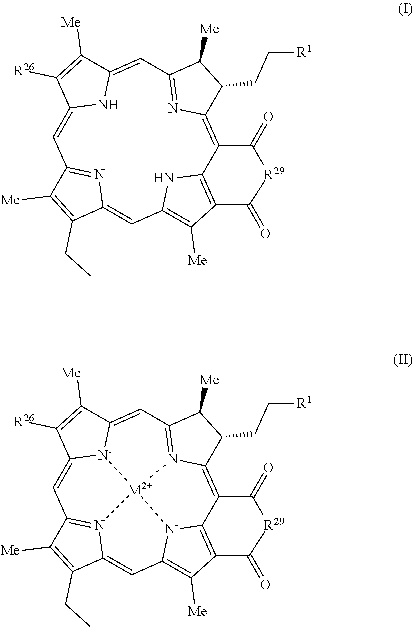
or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[LP(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)RR, —R^{α}—S(O)₂R^{β}, —R^{α}—NH₂, —R^{α}—NH(R^{β}), —R^{α}—N(R^{β})₂, —R^{c}A, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P or Se in its carbon skeleton;
—R⁵, each independently, is selected from C₁-C₄ alkyl, C₁-C₄ haloalkyl, —(CH₂CH₂)₁—H, —(CH₂CH₂)₁—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —OR², —N(R²)₂, —SR², —S(O)R², —S(O)₂R², or —X;
—R⁷ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{7′} is —[NC₅H₅] substituted with —CO₂⁻ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁹ is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R—N(R^{β})₂, —R^{α}—X, —R^{α}—[N(R⁵)₃]Y, —R^{α}—[P(R⁵)₃]Y, —R^{α}—[R⁷]Y, —R^{α}—[N(R⁵)₂(R^{5′})], —R^{α}—[P(R⁵)₂(R^{5′})] or —R^{α}—[R^{7′}];
—R²⁶ is selected from —CH₂R⁶, —CH₂CH₃, —CH═CH₂ or —C(O)H;
—R²⁹— is selected from —N(R⁹)—, —N(OR⁹)—, —O—, —S— or —Se—;
n is 1, 2, 3, 4, 5 or 6;
X is a halo group;
Y is a counter anion;
Z is a counter cation; and
M²⁺ is a metal cation;
provided that the compound of formula (I) or the complex of formula (II) comprises at least one group selected from —R^{α}—[P(R⁵)₃]Y, —R^{α}—[R⁷]Y, —R^{α}—[P(R⁵)₂(R^{5′})] or —R^{α}—[R^{7′}].

2. The compound or complex according to claim 1, wherein each —R^{α}— is independently selected from C₁-C₆ alkylene.

3. The compound or complex according to any preceding claim, wherein —R⁶ is selected from —O—C(O)R¹⁴, —N(R¹²)—C(O)R¹⁴, —O—C(O)—OR¹⁴, —N(R¹²)—C(O)—OR¹⁴, —O—C(O)—N(R¹²)(R¹⁴), or —N(R¹²)—C(O)—N(R¹²)(R¹⁴); —R¹² is selected from hydrogen or C₁-C₃ alkyl; —R¹⁴ is selected from —R^{α}—[P(R⁵)₃]Y, —R^{α}—[R⁷]Y, —R^{α}—[P(R⁵)₂(R^{5′})] or —R^{α}—[R^{7′}]; each —R⁵ is independently selected from C₁-C₄ alkyl or phenyl wherein the phenyl is optionally substituted with one, two or three C₁-C₄ alkyl or C₁-C₄ alkoxy groups; each —R^{5′} is selected from C₁-C₄ alkyl or phenyl, each substituted with —COY, wherein the phenyl is optionally further substituted with one, two or three C₁-C₄ alkyl or C₁-C₄ alkoxy groups; —R⁷ is —[NC₅H₅] optionally substituted with one, two or three C₁-C₄ alkyl or C₁-C₄ alkoxy groups; —R^{7′} is —[NC₅H₅] substituted with —COY and optionally further substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion.

4. The compound or complex according to any preceding claim, wherein —R¹ is —C(O)—OR³, —R³ is —Re, and —R^{β} is a C₁-C₄ alkyl group.

5. The compound or complex according to claim 1, wherein the compound or complex is: 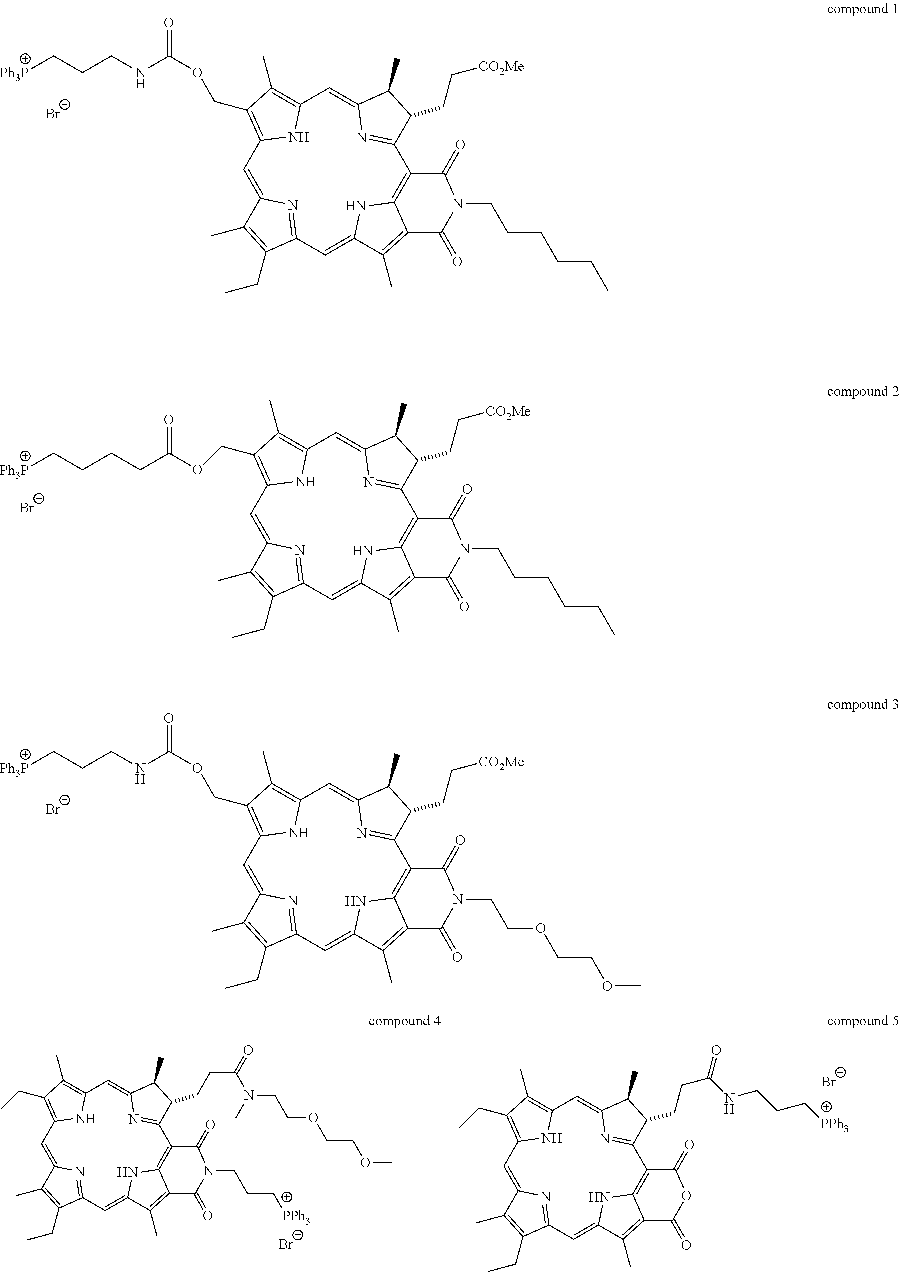 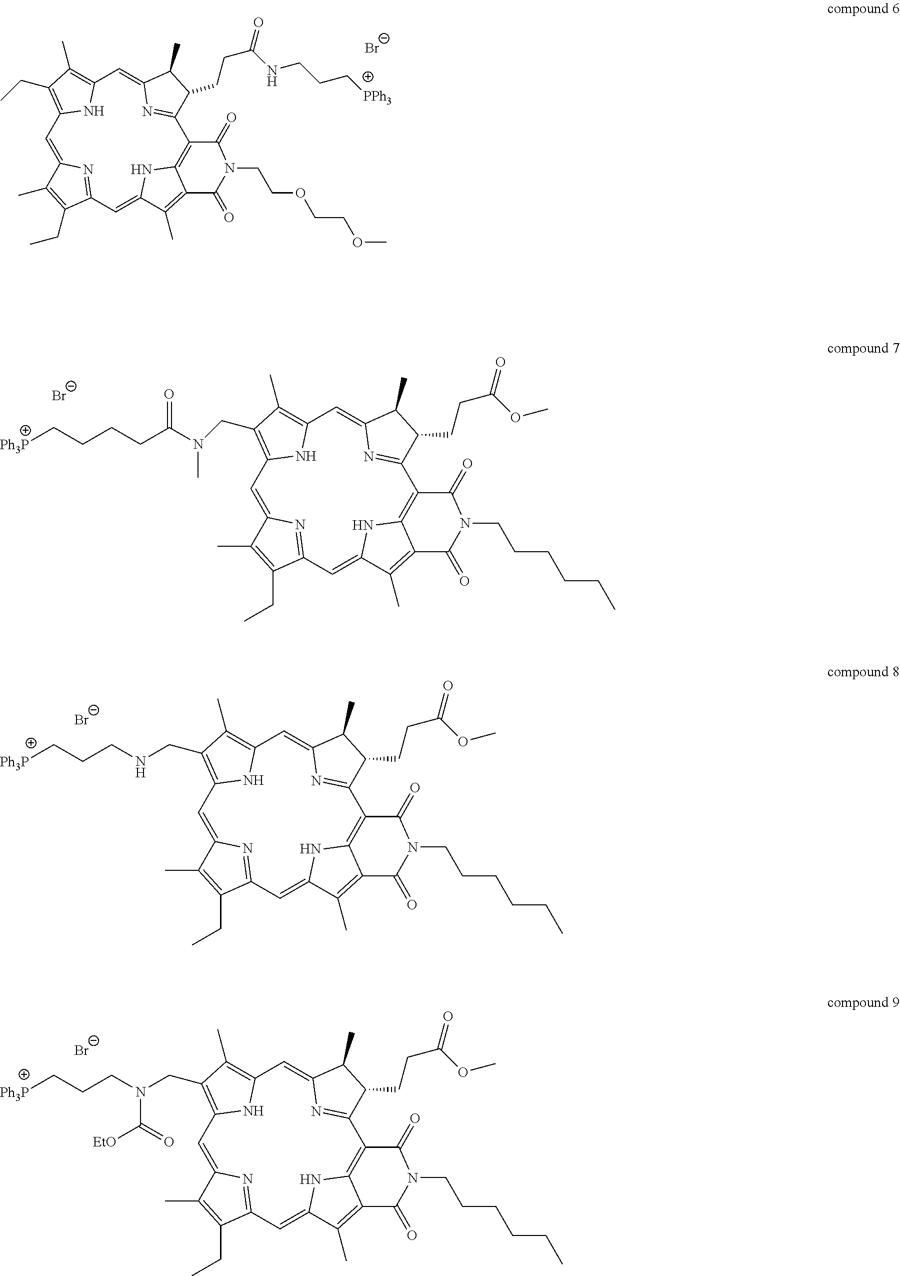 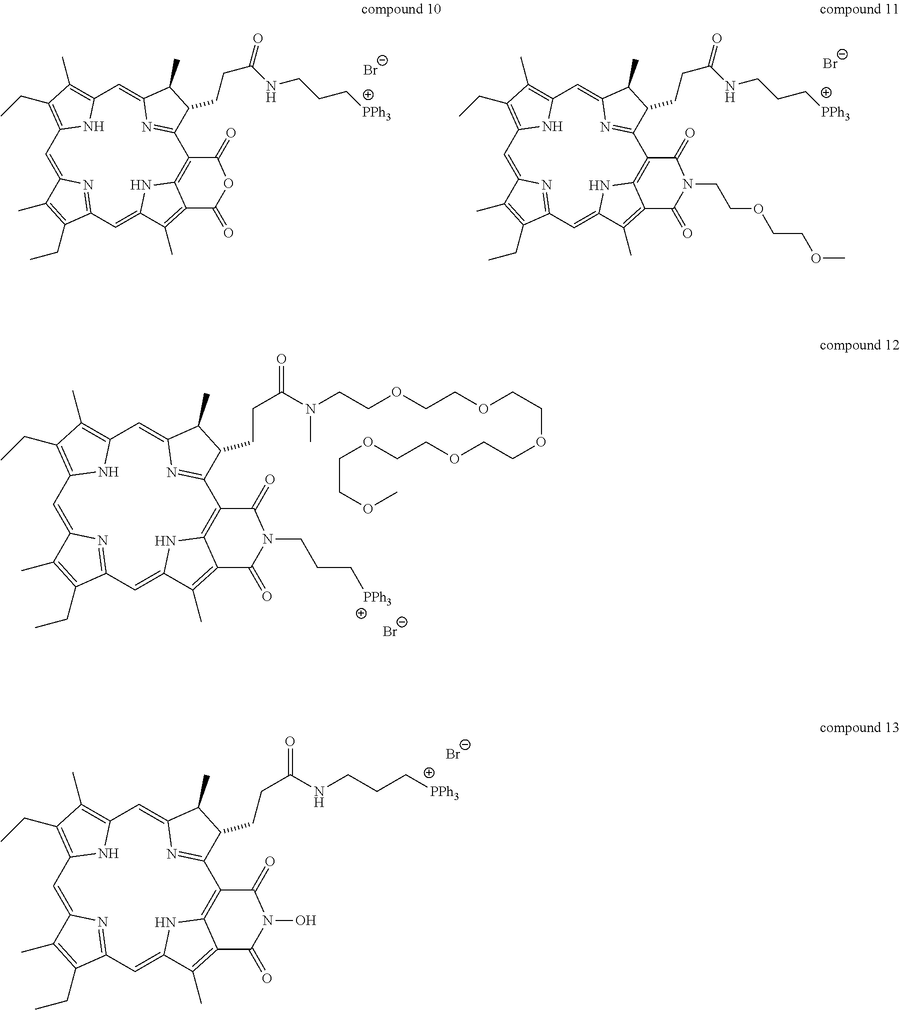
or a metal cation complex thereof, or a pharmaceutically acceptable salt thereof.

6. The compound or complex according to any preceding claim, for use in medicine.

7. The compound or complex according to any preceding claim, for use in photodynamic therapy or cytoluminescent therapy.

8. The compound or complex according to any preceding claim, for use in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

9. The compound or complex according to any preceding claim, for use in the treatment of a disease characterised by benign or malignant cellular hyperproliferation or by areas of neovascularisation.

10. The compound or complex according to any preceding claim, for use in the treatment of a benign or malignant tumour.

11. The compound or complex according to any preceding claim, for use in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

12. The compound or complex according to any preceding claim, for use in photodynamic diagnosis.

13. The compound or complex according to any preceding claim, wherein the compound is adapted for administration prior to administration of irradiation.

14. The compound or complex according to claim 13, wherein the irradiation is electromagnetic radiation with a wavelength in the range of from 500 nm to 1000 nm.

15. A pharmaceutical composition comprising a compound or complex according to any preceding claim and a pharmaceutically acceptable carrier or diluent.

16. The pharmaceutical composition according to claim 15, further comprising polyvinylpyrrolidone.

17. The pharmaceutical composition according to claim 15 or 16, further comprising an immune checkpoint inhibitor.

18. The pharmaceutical composition according to claim 17, wherein the immune checkpoint inhibitor is selected from Pembrolizumab, Nivolumab, Cemiplimab, Atezolizumab, Avelumab, Durvalumab or Ipilimumab.

19. The pharmaceutical composition according to any one of claims 15-18, wherein the pharmaceutical composition is in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration.

20. The pharmaceutical composition according to claim 19, wherein the pharmaceutical composition is in a form suitable for oral or parenteral administration.

21. A pharmaceutical combination or kit comprising:
(a) a compound or complex according to any one of claims 1-14; and
(b) a co-agent which is an immune checkpoint inhibitor.

22. The pharmaceutical combination or kit according to claim 21, wherein the immune checkpoint inhibitor is selected from Pembrolizumab, Nivolumab, Cemiplimab, Atezolizumab, Avelumab, Durvalumab or Ipilimumab.
